SEQUENCE LISTING (33 sequences)

SEQ ID NO: 1 - protein, 481 aa, Human
MAQTQGTRRKVCYYYDGDVGNYYYGQGHPMKPHRIRMTHNLLLNYGLYRKMEIYRPHKANAEEMTKYHSDDYIKFLRSIRPDNMSEYSKQMQRFNVGEDCPVFDGLFEFCQLSTGGSVASAVKLNKQQTDIAVNWAGGLHHAKKSEASGFCYVNDIVLAILELLKYHQRVLYIDIDIHHGDGVEEAFYTTDRVMTVSFHKYGEYFPGTGDLRDIGAGKGKYYAVYPLRDGIDDESYEAIFKPVMSKVMEMFQPSAVVLQCGSDSLSGDRLGCFNLTIKGHAKCVEFVKSFNLPMLMLGGGGYTIRNVARCWTYETAVALDTEIPNELPYNDYFEYFGPDFKLHISPSNMTNQNTNEYLEKIKQRLFENLRMLPHAPGVQMQAIPEDAIPEESGDEDEDDPDKRISICSSDKRIACEEEFSDSEEEGEGGRKNSSNFKKAKRVKTEDEKEKDPEEKKEVTEEEKTKEEKPEAKGVKEEVKLA

SEQ ID NO: 2 - DNA, 1611 bp, Human
atgtctggggtctctgcccgctggtgctgctgtctcccactcggtcatcctgagaacacagcctgagcgrctctgtcactcggggtagaccacgcggggaggcgagcaagatggcgcagacgcagggcacccggaggaaagtctgttactactacgacggggatgttggaaattactattatggacaaggccacccaatgaagcctcaccgaatccgcatgactcataatttgctgctcaactatggtctctaccgaaaaatggaaatctatcgccctcacaaagccaatgctgaggagatgaccaagtaccacagcgatgactacattaaattcttgcgctccatccgtccagataacatgtcggagtacagcaagcagatgcagagattcaacgttggtgaggactgtccagtattcgatggcctgtttgagttctgtcagttgtctactggtggttctgtggcaagtgctgtgaaacttaataagcagcagacggacatcgccgtgaattgggctgggggcctgcaccatgcaaagaagtccgaggcatctggcttctgttacgtcaatgatatcgtcttggccatcctggaactgctaaagtatcaccagagggtgctgtacattgacattgatattcaccatggtgacggcgtggaagaggccttctacaccacggaccgggtcatgactgtgtcctttcataagtatggagagtacttcccaggaactggggacctacgggataccggggctggcaaagacaagtattatgctgttaactacccgctccgagacgggattgatgacgagtcctatgaggccattttcaagccggtcatgtccaaagtaatggagatgttccagcctagtgcggtggtcttacagtgtggctcagactccctatctggggatcggttaggttgcttcaatctatctatcaaaggacacgccaagtgtgtggaatttgtcaagagctttaacctgcctatgctgatgctgggaggcggtggttacaccattcgtaacgttgcccggtgctggacatatgagacagctgtggccctggatacggagatccctaatgagcttccatacaatgactactttgaatactttggaccagatttcaagctccacatcagtccttccaatatgactaaccagaacacgaatgagtacctggagaagatcaaacagcgactgtttgagaaccttagaatgctgccgcacgcacctggggtccaaacgcaggcgattcctgaggacgccatccctgaggagagtggcgatgaggacgaagacgaccctgacaagcgcatctcgatctgctcctctgacaaacgaattgcctgtgaggaagagttctccgattctgaagaggagggagaggggggccgcaagaactcttccaacttcaaaaaagccaagagagtcaaaacagaggatgaaaaagagaaagacccagaggagaagaaaggaatcaccgaagaggagaaaaccaaggaggagaagccagaagccaaaggggtcaaggaggaggccaagttggcctgaatggacctctccagctctggcttcctgctgagtccctcacgtttctttcccc

SEQ ID NO: 3 - protein, 489 aa, Human
MAYSQGGGKKKCKVCYYYDGDIGNYYYGQGHPMKPHRIRMTHNLLLNYGLYRKMEIYRPHKATAEEMTKYHSDEYIKFLRSIRPDNMSEYSKQMHIPFNVGEDCPAFDGLFEFCQLSTGGSVAGAVKLNRQQTDMAVNWAGGLHHAKKYEASGFCYVNDIVLAILELLKYHQRVLYIDIDIHHRGDGVEEAFYTTDRVMTVSFYGEYFPGTGDLRDIGAGKGKYYAVNFPMCDGIDDESYGQIFKPIISKVMEMYQPSAVVLQCGADSLSGDRLGCFNLTVKGHAKCVEVVKTFNLPLLMLGGGGYTILRNVARCWTYETAVALDCEIPNELPYNDYFEYFGPDFKLHISPSNMTNQNTPEYMEKIKQRLFENLRMLPHAPGVQMQAIPEDAVHEDSGDEDGEDPDKRISIRASDKRIACDEEFSDSEDEGEGGRNVADHKKGAKARIEEDKKETEDKKTDVKEEDKSKDNSGEKTDTKGTKSEQLSNP

SEQ ID NO: 4 - DNA, 1985 bp, Human
cgccgagctttcggcacctctgccgggtggtaccgagccttcccggcgccccctcctctcctcccaccggcctgcccttccccgcgggactatcgcccccacgtttccctcagcccttttctctcccggccgagccgcggcggcagcagcagcagcagcagcagcaggaggaggagcccggtggcggcggtggccggggagcccatggcgtacagtcaaggaggcggcaaaaaaaaagtctgctactactacgacggtgatattggaaattattattatggacagggtcatcccatgaagcctcatagaatccgcatgacccataacttgctgttaaattatggcttacacagaaaaatggaaatatataggccccataaagccactgccgaagaaatgacaaaatatcacagtgatgagtatatcaaatttctacggtcaataagaccagataacatgtctgagtatagtaagcagatgcatatatttaatgttggagaagattgtccagcgtttgatggactctttgagttttgtcagctctcaactggcggttcagttgctggagctgtgaagttaaaccgacaacagactgatatggctgttaattgggctggaggattacatcatgctaagaaatacgaagcatcaggatcctgttacgttaatgatattgtgcttgccatccttgaattactaaagtatcatcagagagtcttatatatcgatatagatattcatcatggtgatggtgtcgaagaagctttttatacaacagatcgtgtaatgacggtatcattccataaatatggggaatactttcctggcacaggagacttgagggatattggtgctggaaaaggcaaatactatgctgtcaattttccaatgtgtgatggtatagacgatgagtcatatgggcagatatttaagcctattatctcaaaggtgatggagatgtatcaacctagtgctgtggtattacagtgtggtgcagactcattatctggtgatagactgggttgtttcaatctaacagtcaaaggtcatgctaaatgtgtagaagttgtaaaaacttttaacttaccattactgatgcttggaggaggtggctacacaatccgtaatgttgctcgatgttggacatatgagactgcagttgcccttgattgtgagattcccaacgagttgccatataatgattactttgagtattttggaccagacttcaaactgcatattagtccttcaaacatgacaaaccagaacactccagaatatacggaaaagataaaacagcgtttgtttgaaaatttgcgcatgttacctcatgcacctggtgtccagatgcaagctattccagaagatgctgttcatgaagacagtggagatgaagatggagaagatccagacaagagaatttctattcgagcatcagacaagcggatagcttgtgatgaagaattctcagattctgaggatgaaggagaaggaggtcgaagaaatgtggctgatcataagaaaggagcaaagaaagctagaattgaagaagataagaaagaaacagaggacaaaaaaacagacgttaaggaagaagataaatccaaggacaacagtggtgaaaaaacagataccaaaggaaccaaatcagaacagctcagcaacccctgaatctgacagtctcaccaatttcagaaaatcattaaaaagaaaatattgaaaggaaaatgttttctttttgaagacttctggcttcattttatactactttggcatggactgtatttattttcaaatgggactttttcgtttttgtttttctgggcaagttttattgtgagattttctaattatgaagcaaaatttcttttctccaccatgctttatgtgatagtatttaaaattgatgtgagttattatgtcaaaaaaactgatctattaaagaagtaattggcctttctgagctgaaaaaaaaaaaaaaaaag

SEQ ID NO: 5 - protein, 428 aa, Human
MAKTVAYFYDPDVGNFHYGAGHPMKPHRLALTHSLVLHYGLYKKMIVFKPYQASQHDMCRFHSEDYIDFLQRVSPTNMQGFTKSLNAPNVGDDCPVFPGLFEFCSRYTGASLQGATQLNNKICDIANWAGGLHHAKKFEASGFCYVNDIVIGILELLLKYHPRVLYIDIDIHHGDGVQEAFYLTDRVMTVSFHKYGNYFFPGTGDMYEVGAESGRYYCLNVPLRDGIDDQSYKHLFQPVINQVVDFYQPTCIVLQCGADSLGCDRLGCFNLSIRGHCECVEYVKSFNIPPLLVLGGGGYTVRNVARCWTYETSLLVEEAISEELPYSEYFEYFAPDFTLHPDVSTRIENQSRQYLDQIRQTIFENLKMLNHAPSVQIHDVPADLLTYDRTDEADAEERGPEENYSRPEAPNEFYDGDHDNDKESDVEI

SEQ ID NO: 6 - DNA, 1954 bp, Human
ggaattcgcggccgcggcgggcgcgggaggtgcggggcctgctcccgccggcaccatggccaagaccgtggcctatttctacgaccccgacgtgggcaacttccactacggagctggacaccctatgaagccccatcgcctggcattgacccatagcctggtcctgcattacggtctctataagaagatgatcgtcctcaagccataccaggcctcccaacatgacatgtgccgcttccactccgaggactacattgacttcctgcagagagtcagccccaccaatatgcaaggcttcaccaagagtcttaatgccttcaacgtaggcgatgactgcccagtgtttcccgggctctttgagttctgctcgcgttacacaggcgcatctctgcaaggagcaacccagctgaacaacaagatctgtgatattgccattaactgggctggtggtctgcaccatgccaagaagtttgaggcctctggcttctgctatgtcaacgacattgtgattggcatcctggagctgctcaagtaccaccctcgggtgctctacattgacattgacatccaccatggtgacggggttcaagaagctttctacctcactgaccgggtcatgacggtgtccttccacaaatacggaaattacttcttccctggcacaggtgacatgtatgaagtcggggcagagagtggccgctactactgtctgaacgtgcccctgcgggatggcattgatgaccagagttacaagcaccttttccagccggttatcaaccaggtagtggacttctaccaacccacgtgcattgtgctccagtgtggagctgactctctgggctgtgatcgattgggctgctttaacctcagcatccgagggcatggggaatgcgttgaatatgtcaagagcttcaatatccctctactcgtgctgggtggtggtggttatactgtccgaaatgttgcccgctgctggacatatgagacatcgctgctggcagaagaggccattagtgaggagcttccctatagtgaatacttcgagtactttgccccagacttcacacttcatccagatgtcagcacccgcatcgagaatcagaactcacgccagtatctggaccagatccgccagacaatctttgaaaacctgaagatgctgaaccatgcacctagtgtccagattcatgacgtgcctgcagacctcctgacctacgacaggaccgatgaggccgatgcagaggagaggggtcctgaggagaactatagcaggccagaggcatccaatgagttctatgatggagaccatgacaatgacaaggaaagcgatgtggagatttaagagtggcttgggatgctgtgtcccaaggaatttcttttcacctcttggaagggctggagggaaaaggagtggctcctagagtcctgggggtcaccccaggggcttttgctgactctgggaaagagtctggagaccacatttggttctcgaaccatctacctgcttttcctctctctcccaaggactgacaatggtacctattagggatgagatacagacaaggatagctatctgggacattattggcagtgggccctggaggcagtccctagccccccttgccccttatttcttccctgcttccctcgaacccagagatttttgagggatgaacgggtagacaaggactgagattgcctctgacttcctcctcccctgggttctgaccttcttcctccccttgcttccagggaagatgaagagagagagatttggaaggggctctggctccctaacacctgaatcccagatgatgggaagtatgttttcaagtgtggggaggatatgaaaatgttctgctctcacttttggctttatgtccattttaccactgtttttatccaataaactaagtcggtattttttgtacctttgatggtttagcggccgcgc

SEQ ID NO: 7 - protein, 967 aa, Human
MLAMKHQQELLEHQRKLERHRQEQELEKQHREQKLQQLKNKEKGKESAVASTEVKMKLQEFVLNKKKALAHPNLNHCISSCPRYWYGKTQHSSLDQSSPPQSGVSTSYNHPVLGMYDAKDDFPLRKTASEPNLKLRSRLKQKVAERRSSPLLRRKDGPVVTALKKRPLDVTDSACSSAPGSGPSSPNNSSGSVSAENGIAPAVPSIPAETSLAHRLVAREGSAAPLPLYTSPSLPNITLGLPATGPSAGTAGQQDTERLTLPALQQRLSLFPGTHLTPYLSTSPLERDGGAAHSPLLQHMVLLEQPPAQAPLVTGLGALPLHAQSLVGADRVSPSIHKLRQHRPLGRTQSAPLPQNAQALQHLVIQQQHQQFLEKHKQQFQQQQLQMNKIIPKPSEPARQPESHPEETEEELREHQALLDEPYLDRLPGQKEAHAQAGVQVKQEPIESDEEEAEPPREVEPGQRQPSEQELLFRQQALLLEQQRIHQLRNYQASMEAAGIPVSFGGHRPLSRAQSSPASATFPVSVQEPPTKPRFTTGLVYDTLMLKHQCTCGSSSSHPEHAGRIQSIWSRLQETGLRGKCECIRGRKATLEELQTVHSEAHTLLYGTNPLNRQKLDSKKLLGSLASVFVRLPCGGVGVDSDTIWNEVHSAGAARLAVGCVVELVFKVATGELKNGFAVVRPPGHHAEESTPMGFCYFNSVAVAAKLLQQRLSVSKILIVDWDVHHGNGTQQAFYSDPSVLYMSLHRYDDGNFFPGSGAPDEVGTGPGVGFNVNMAFTGGLDPPMGDAEYLAAFRTVVMPIASEFAPDVVLASSGFDAVEGHPTPLGGYNLSARCFGYLTKQLMGLAGGRIVLALEGGHDLTAICDASEACVSALLGNELDPLPEKVLQQRPNANAVRSMEKVMEIHSKYWRCLQRTTSTAGRSLIEAQTCENEEAETVTAMASLSVGVKPAEKRPDEEPMEEEPPL

SEQ ID NO: 8 - DNA, 8459 bp, Human
ggaggttgtggggccgccgccgcggagcaccgtccccgccgccgcccgagcccgagcccgagcccgcgcacccgcccgcgccgccgccgccgccgcccgaacagcctcccagcctgggcccccggcggcgccgtggccgcgtcccggctgtcgccgcccgagcccgagcccgcgcgccggcgggtggcggcgcaggctgaggagatgcggcgcggagcgccggagcagggctagagccggccgccgccgcccgccgcggtaagcgcagccccggcccggcgcccgcgggccattgtccgccgcccgccccgcgccccgcgcagcctgcaggccttggagcccgcggcaggtggacgccgccggtccacacccgccccgcgcgcggccgtgggaggcgggggccagcgctggccgcgcgccgtgggacccgccggtccccagggccgcccggccccttctggacctttccacccgcgccgcgaggcggcttcgcccgccggggcgggggcgcgggggtgggcacggcaggcagcggcgccgtctcccggtgcggggcccgcgccccccgagcaggttcatctgcagaagccagcggacgcctctgttcaacttgtgggttacctggctcatgagaccttgccggcgaggctcggcgcttgaacgtctgtgacccagccctcaccgtcccggtacttgtatgtgttggcgggagtttggagctcgttggagctatcgtttccgtggaaattttgagccatttcgaatcacttaaaggagtggacattgctagcaatgagctcccaaagccatccagatggactttctggccgagaccagccagtggagctgctgaatcccgcccgcgtgaaccacatgcccagcacggtggatgtggccacggcgctgcctctgcaagtggcccccccggcagtgcccatggacccgcgcctggaccaccagttctcactgcctgtggcagagccggccctgcgggagcagcagctgcagcaggagctcctggcgctcaagcagaagcagcagatccagaggcagatcctcatcgccgagttccagaggcagcacgagcagctctcccggcagcacgaggcgcagctccacgagcacatcaagcaataacaggagatgctggccatgaagcaccagcaggagctgctggaacaccagcggaagctggagaggcaccgccaggagcaggagctggagaagcagcaccgggagcagaagctgcagcagctcaagaacaaggagaagggcaaagagagtgccgtggccagcacagaagtgaagatgaagttacaagaatttgtcctcaataaaaagaaggcgctggcccaccggaatctgaaccactgcacttccagagaccctcgctactggtacgggaaaacgcagcacagttcccttgaccagagttctccaccccagagcggagtgtcgacctcctataaccacccggtcctgggaatgtacgacgccaaagatgacttccctcttaggaaaacagcttctgaaccgaatctgaaatcacggtccaggctaaagcagaaagtggccgaaagacggagcagccccctgttacgcaggaaagacgggccagtggtcactgctctaaaaaagcgtccgttggatgtcacagactccgcgtgcagcagcgccccaggctccggacccagctcacccaacaacagctccgggagcgtcgcgtggaggaacggtatcgcgcccgccgtccccagcatcccggcggagacgagtttggcgcacagacttgtggcacgagaaggctcggccgctccacttcccctctacacatcgccatccttgcccaacatcacgctgggcctgcctgccaccggcccctctgcgggcacggcgggccagcaggacaccgagagactcacccttcccgccctccagcagaggctctcccttttccccggcacccacctcactccctacctgagcacctcgcccttggagcgggacggaggggcagcgcacagccctcttctgcagcacatggtcttactggagcagccaccggcacaagcacccctcgtcacaggcctgggagtactgcccctccacgcacagtccttggttggtgcagaccgggtgtccccctccatccacaagctgcggcagcaccgcccactggggcggacccagtcggccccgctgccccagaacgcccaggctctgcagcacctggtcatccagcagcagcatcagcagtttctggagaaacacaagcagcagttccagcagcagcaactgcagatgaacaagatcatccccaagccaagcgagccagcccggcagccggagagccacccggaggagacggaggaggagctccgtgagcaccaggctctgctggacgagccctacctggaccggctgccggggcagaaggaggcgcacgcacaggccggcgtgcaggtgaagcaggagcccattgagagcgatgaggaagaggcagagcccccacgggaggtggagccgggccagcgccagcccagtgagcaggagctgctcttcagacagcaagccctcctgctggagcagcagcggatccaccagctgaggaactaccaggcgtccatggaggccgccggcatccccgtgtccttcggcggccacaggcctctgtcccgggcgcagtcctcacccgcgtctgccaccttccccgtgtccgtgcaggagccccccaccaagccgaggttcacgacaggcctcgtgtatgacacgctgatgctgaagcaccagtgcacctgcgggagtagcagcagccaccccgagcacgccgggaggatccagagcatctggtcccgcctgcagaagacgggcctccggggcaaatgcgagtgcatccgcggacgcaaggccaccctggaagagctacagacggtgcactcggaagcccacaccctcctgtatggcacgaaccccctcaaccggcagaaactggacagtaagaaacttctaggctcgctcgcctccgtgttcgtccggctcccttgcggtggtgttggggtggacagtgacaccatatggaacgaggtgcactcggcgggggcagcccgcctggctgtgggctgcgtggtagagctggtcttcaaggtggccacaggggagctgaaaaatggctttgctgtggtccgcccccctggacaccatgcggaggagagcacgcccatgggcttttgctacttcaactccgcggccgtggcagccaagcttctgcagcagaggttgagcgtgagcaagatcctcatcgtggactgggacgtgcaccatggaaacgggacccagcaggctttctacagcgaccctagcgtcctgtacatgtccctccaccgctacgacgatgggaacttcttcccaggcagcggggctcctgatgaggtgggcacagggcccggcgtgggtttcaacgtcaacacggctttcaccggcggcctggacccccccatgggagacgctgagtacttggcggccttcagaacggtggtaatgccgatcgccagcgagtttgccccggatgtggtgctggtgtcatcaggcttcgatgccgtggagggccaccccacccctcttgggggctacaacctctccgccagatgcttcgggtacctgacgaagcagctgatgggcctggctggcggccggattgtcctggccctcgagggaggccacgacctgaccgccatttgcgacgcctcggaagcatgtgtttctgccttgctgggaaacgagcttgatcctctcccagaaaaggttttacagcaaagacccaatgcaaacgctgtccgttccatggagaaagtcatggagatccacagcaagtactggcgctgcctgcagcgcacaacctccacagcggggcgttctctgatcgaggctcagacttgcgagaacgaagaagccgagacggtcaccgccatggcctcgctgtccgtggacgtgaagcccgccgaaaagagaccagatgaggagcccatggaagaggagccgcccctgtagcactccctcgaagctgctgttctcttgtctgtctgtctctgtcttgaagctcagccaagaaactttcccgtgtcacgcctgcgtcccaccgtggggctctcttggagcacccagggacacccagcgtgcaacagccacgggaagcctttctgccgcccaggcccacaggtctcgagacgcacatgcacgcctgggcgtggcagcctcacagggaacacgggacagacgccggcgacgcgcagacacacggacacgcggaagccaagcacactctggcgggtcccgcaagggacgccgtggaagaaaggagcctgtggcaacaggcggccgagctgccgaattcagttgacacgaggcacagaaaacaaatatcaaagatctaataatacaaaacaaacttgattaaaactggtgcttaaagtttattacccacaactccacagtctctgtgtaaaccactcgactcatcttgtagcttattttttttttaaagaggacgttttctacggctgtggcccgcctctgtgaaccatagcggtgtgcggcggggggtctgcacccgggtgggggacagagggacctttaaagaaaacaaaactggacagaaacaggaatgtgagctgggggagctggcttgagtttctcaaaagccatcggaagatgcgagtttgtgcctttttttttattgctctgtcacttggtcactgggctgctgatggtcagctctgagacagtggtttgagagcaggcagagtggatttttgtggctgggttttctgaagtctgaggaacaatgccttaagaaaaaacaaacagcaggaatcggtgggacagtttcctgtggccagccgagcctggcagtgctggcaccgcgagctggcctgacgcctcaagcacgggcaccagccgtcatctccggggccaggggctgcagcccggcggtccctgttttgctttattgctgtttaagaaaaatggaggtagttccaaaaaagtggcaaatcccgttggaggttttgaagtccaacaaattttaaacgaatccaaagtgttctcacacgtcacatacgattgagcatctccatctggtcgtgaagcatgtggtaggcacacttgcagtgttacgatcggaatgctttttattaaaagcaagtagcatgaagtattgcttaaattttaggtataaataaatatatatatgtataatatatattccaatgtattccaagctaagaaacttacttgattcttatgaaatcttgataaaatatttataatgcatttatagaaaaagtatatatatatatataaaatgaatgcagattgcgaaggtccctgcaaatggatggcttgtgaatttgctctcaaggtgcttatggaaagggatcctgattgattgaaattcatgttttctcaagctccagattggctagatttcagatcgccaacacattcgccactgggcaactaccctacaagtttgtactttcattttaattattttctaacagaaccgctcccgtctccaagccttcatgcacatatgtacctaatgagtttttatagcaaagaatataaatttgctgttgatttttgtatgaattttttcacaaaaagatcctgaataagcattgttttatgaattttacatttttcctcaccatttagcaattttccgaatggtaataatgtctaaatctttttcctttctgaattcttgcttgtacatttttttttacctttcaaaggtttttaattatttttgtttttatttttgtacgatgagttttctgcagcgtacagaattgttgctgtcagattctattttcagaaagtgagaggagggaccgtaggtcttttcggagtgacaccaacgattgtgtctttcctggtctgtcctaggagctgtataaagaagcccaggggctctttttaactttcaacactagtagtattacgaggggtggtgtgtttttcccctccgtggcaagggcagggagggttgcttaggatgcccggccaccctgggaggcttgccagatgccgggggcagtcagcattaatgaaactcatgtttaaacttctctgaccacatcgtcaggatagaattctaacttgagttttccaaacaccttttgagcatgtcagcaatgcatggggcacacgtggggctctttacccacttgggtttttccactgcagccacgtggccagccctggattttggagcctgtggctgcaaggaacccagggacccttgttgcctggtgaacctgcagggagggtatgattgcctgaccaggacagccagtctttactctttttctcttcaacagtaactgacagtcacgttttactggtaacttattttccagcacatgaagccaccagtttcattccaaagtgtatattgggttcagacttgggggcagaagttcagacacaccgtgctcaggagggacccagagccgagtttcggagtttggtaaagtttacagggtagcttctgaaattaactcaaacttttgaccaaatgagtgcagattcttggatacggtcttgggacttgtttgactttcccctccctggtggccactctttgctctgaagcccagattggcaagaggagctggtccattccccattcatggcacagaacagtggcagggcccagctagcaggctcttctggcctccttggcctcattctctgcatagccctctggggatcctgccacctgccctcttaccccgccgtggcttatggggaggaatgcatcatctcactttttttttttaagcagatgatgggataacatggactgctcagtggccaggttatcagtggggggacttaattctaatctcattcaaatggagacgacctctgcaaaggcctggcagggggaggcaagtttcatctgtcagctcactccagcttcacaaatgtgctgagagcattactgtgtagccttttctttgaagacacactcggctcttctccacagcaagcgtccagggcagatggcagaggatctgcctcggcgtctgcaggcgggaccacgtcagggagggttccttcatgtgttctccctgtgggtccttggacctttagcctttttcttcctttgcaaaggccttgggggcactggctgggagtcagcaagcgagcactttatatccctttgagggaaaccctgatgacgccactgggcctcttggcgtctgacctgccctcgccgcttcccgccgtgccgcagcgtgcccacgtgcccacgccccaccagcaggcggctgccccggaggccgtggcccgctgggactggccgcccctccccagcgtcccagggctctggttctggagggccactttgtcaaggtgtttcagtttttctttacttcttttgaaaatctgtttgcaaggggaaggaccatttcgtaatggtctgacacaaaagcaagtttgatttttgcagcactagcaatggactttgttgcttttctttttgatcagaacattccttctttactggtcacagccacgtgctcattccattcttctttttgtagactttgggcccacgtgttttatgggcattgatacatatataaatatatagatataaatatatatgaatacatttttttaagtttcctacacctggaggttgcatggactgtacgaccggcatgactttatattgtatacagattttgcacgccaaactcggcagctttggggaagaagaaaaatgcctttctgttcccctctcatgacatttgcagatacaaaagatggaaatttttctgtaaaacaaaaccttgaaggagaggagggcggggaagtttgcgtcttattgaacttattcttaagaaattgtactttttattgtaagaaaaataaaaaggactacttaaacatttgtcatattaagaaaaaaagtttatctagcacttgtgacataccaataatagagtttattgtatttatgtggaaacagtgttttagggaaactactcagaattcacagtgaactgcctgtctctctcgagttgatttggaggaattttgttttgttttgttttgtttgtttccttttatctccttccacgggccaggcgagcgccgcccgccctcactggccttgtgacggtttattctgattgagaactgggcggactcgaaagagtccccttttccgcacagctgtgttgactttttaattacttttaggtgatgtatggctaagatttcactttaagcagtcgtgaactgtgcgagcactgtggtttacaattatactttgcatcgaaaggaaaccatttcttcattgtaacgaagctgagcgtgttcttagctcggcctcactttgtctctggcattgattaaaagtctgctattgaaagaaaaag

SEQ ID NO: 9 - protein, 717 aa, Human
LRQGGTLTGKFMSTSSIPGCLLGVALEGDGSPHGHASLLQHVLLLEQARQQSTLIAVPLHGQSPLVTGERVATSMRTVGKLPRHRPLSRTQSSPLPQSPQALQQLVMQQQHQQFLEKQKQQQLQLGKILTKTGELPRQPTTHPEETEEELTEQQEVLLGEGALTMPREGSTESESTQEDLEEEDEEEDGEEEDCIQVKDEEGESGAEEGPDLEEPGAGYKKLFSDAQPLQPLQVYQAPLSLATVPHQALGRTQSSPAAPGGMKSPPDQPVKHLFTTGVVYDTFMLKHQCMCGNTHVHPEHAGRIQSIWSRLQETGLLSKCERIRGRKATLDEIQTVHSEYIHTLLYGTSPLNRQKLDSKKLLGPISQKMYAVLPCGGIGVDSDTVWNEMHSSSAVRMAVGCLLELAFKVAAGELKNGFAIIRPPGHHAEESTAMGFCFFNSVAITAKLLQQKLNVGKVLIVDWDIHHGNGTQQAFYNDPSVLYISLHRYDNGNFFPGSGAPEEVGGGPGVGYNVNVAWTGGVDPPIGDVEYLTAFRTVVMPIAHEFSPDVVTLVSAGFDAVEGHLSPLGGYSVTARCFGHLTRQLMTLAGGRVVLALEGGHDLTAICDASEACVSALLSVELQPLDELVLQQKPNINAVATLEKVIETQSKHWSCVQKFAAGLGRSLREAQAGETEEAETVSAMALLSVGAEQAQAAAAREHSPRPAEEPMEQEPAL

SEQ ID NO: 10 - DNA, 2233 bp, Human
ccctgcggcagggtggcacgctgaccggcaagttcatgagcacatcctctattcctggctgcctgctgggcgtggcactggagggcgacgggagcccccacgggcatgcctccctgctgcagcatgtgctgttgctggagcaggcccggcagcagagcaccctcattgctgtgccactccacgggcagtccccactagtgacgggtgaacgtgtggccaccagcatgcggacggtaggcaagctcccgcggcatcggcccctgagccgcactcagtcctcaccgctgccgcagagtccccaggccctgcagcagctggtcatgcaacaacagcaccagcagttcctggagaagcagaagcagcagcagctacagctgggcaagatcctcaccaagacaggggagctgcccaggcagcccaccacccaccctgaggagacagaggaggagctgacggagcagcaggaggtcttgctgggggagggagccctgaccatgccccgggagggctccacagagagtgagagcacacaggaagacctggaggaggaggacgaggaagaggatggggaggaggaggaggattgcatccaggttaaggacgaggagggcgagagtggtgctgaggaggggcccgacttggaggagcctggtgctggatacaaaaaactgttctcagatgcccagccgctgcagcctttgcaggtgtaccaggcgcccctcagcctggccactgtgccccaccaggccctgggccgtacccagtcctcccctgctgcccctgggggcatgaagagccccccagaccagcccgtcaagcacctcttcaccacaggtgtggtctacgacacgttcatgctaaagcaccagtgcatgtgcgggaacacacacgtgcaccctgagcatgctggccggatccagagcatctggtcccggctgcaggagacaggcctgcttagcaagtgcgagcggatccgaggtcgcaaagccacgctagatgagatccagacagtgcactctgaataccacaccctgctctacgggaccagtcccctcaaccggcagaagctagacagcaagaagttgctcggccccatcagccagaagatgtatgctgtgctgccttgtgggggcatcggggtggacagtgacaccgtgtggaatgagatgcactcctccagtgctgtgcgtatggcagtgggctgcctgctggagctggccttcaaggtggctgcaggagagctcaagaatggatttgccatcatccggcccccaggacaccacgccgaggaatccacagccacgggattctgcttcttcaactctgtagccatcaccgcaaaactcctacagcagaagttgaacgtgggcaaggtcctcatcgtggactgggacattcaccatggcaatggcacccagcaggcgttctataatgacccctctgtgctctacatctctctgcatcgctatgacaacgggaacttctttccaggctctggggctcctgaagaggttggtggaggaccaggcgtggggtacaatgtgaacgtggcatggacaggaggtgtggacccccccattggagacgtggagtaccttacagccttcaggacagtggtgatgcccattgcccacgagttctcacctgatgtggtcctagtctccgccgggtttgatgctgttgaaggacatctgtctcctctgggtggctactctgtcaccgccagatgttttggccacttgaccaggcagctgatgaccctggcagggggccgggtggtgctggccctggagggaggccatgacttgaccgccatctgtgatgcctctgaggcttgtgtctcggctctgctcagtgtagagctgcagcccttggatgaggcagtcttgcagcaaaagcccaacatcaacgcagtggccacgctagagaaagtcatcgagatccagagcaaacactggagctgtgtgcagaagttcgccgctggtctgggccggtccctgcgagaggcccaagcaggtgaggccgaggaggccgagactgtgagcgccatggccttgctgtcggtgggggccgagcaggcccaggctgcggcagcccgggaacacagccccaggccggcagaggagcccatggagcaggagcctgccctgtgacgccccggcccccatccctctcggcttcaccattgtgattttgtttattttttctattaaaaacaaaaagtcacacattc

SEQ ID NO: 11 - protein, 1215 aa, Human
MTSTGQDSTTTRQRRSRQNPQSPPQDSSVTSKRNIKKGAVPRSIPNLAEVKKKGKMKKLGQAMEEDLIVGLQGMDLNLEAEALAGTGLVLDEQLNEFHCLWDDSFPEGPERLHAIKEQLIQEGLLDRCVSFQARFAEKEELMLVHSLEYIDLMETTQYMNEGELRVLADTYDSVYLHPNSYSCACLASGSVLRLVDAVLGAEIRNGMAIIRPPGHHAQHSLMDGYCMFNHVAVAARYAQQKHRIRRVLIVDWDVHHGQGTQFTFDQDPSVLYFSIHRYEQGRFWPHLKASNWSTTGFGQGQGYTINVPWNQVGMRDADYIAAFLHVLLPVALEFQPQLVLVAAGFDALQGDPKGEMAATPAGFAQLTHLLMGLAGGKLILSLEGGYNIRALAEGVSASLHTLLGDPCPMLESPGAPCRSAQASVSCALEALEPFWEVLVRSTETVERDNMEEDNVEESEEEGPWEPPVLPILTWPVLQSRTGLVYDQNMMNHCNLWDSHHPEVPQRILRIMCRLEELGIAGRCLTITPRPATEAELLTCHSAEYVGHLRATEKMKTRELHRESSNFDSIYICPSTFACAQIATGAACRLVEAVISGEVINGAAVVRPPGHHAEQDAACGFCFFNSVAVAARHAQTISGHALRILIVDWDVHHGNGTQHMFEDDPSVLYVSLHRYDHGTFFPMGDEGASSQIGRAAGTGFTVNVAWNGPRMGDADYLAAWHRLVLPIAYEFNPELVLVSAGFDAARGDPLGGCQVSPEGYAHLTHLLMGLASGRIILILEGGYNLTSISESMAACTRSILGDPPPLLTLPRPPLSGALASITETIQVHRRYWRSLRVMKVEDREGPSSSKLVTKKAPQPAKPRLAERMTTREKKVLEAGMGKVTSASFGEESTPGQTNSETAVVALCQDQPSEAATGGATLAQTISEAAIGGAMLGQTTSEEAVGGATPDQTTSEETVGGAILDQTTSEDAVGGATIGQTTSEEAVGGATLAQTISEAAMEGATLDQTTSEEAPGGTELIQTPLASSTDHQTPPTSPVQGTTPQISPSTLIGSLRTLELGSESQGASESQAPGEENLLGEAAGGQDMADSMLMQGSRGLTDQAIFYAVTPLPWCPHLVAVCPIPAAGLDVTQPCGDCGTIQENWVCLSCYQVYCGRYINGHMLQHHGNSGHPLVLSYIDLSAWCYYCQAYVHHQALLDVKNIAHQNKFGEDMPHPH

SEQ ID NO: 12 - DNA, 4099 bp, Human
gggcagtcccctgaggagcggggctggttgaaacgctaggggcgggatctggcggagtggaagaaccgcggcaggggccaagcctcctcaactatgacctcaaccggccaggattccaccacaaccaggcagcgaagaagtaggcagaacccccagtcgccccctcaggactccagtgtcacttcgaagcgaaatattaaaaagggagccgttccccgctctatccccaatctagcggaggtaaagaagaaaggcaaaatgaagaagctcggccaagcaatggaagaagacctaatcgtgggactgcaagggatggatctgaacctcgaggctgaagcactggctggcactggcttggtgttggatgagcagttaaatgaattccattgcctctgggatgacagcttcccggaaggccctgagcggctccatgccatcaaggagcaactgatccaggagggcctcctagatcgctgcgtgtcctttcaggcccggtttgctgaaaaggaagagctgatgttggttcacagcctagaatatattgacctgatggaaacaacccagtacatgaatgagggagaactccgtgtcctagcagacacccacgactcagtttatctgcatccgaactcatactcctgtgcctgcctggcctcaggctctgtcctcaggctggtggatgcggtcctgggggctgagatccggaacggcatggccatcattaggcctcctggacatcacgcccagcacagtcttatggatggctattgcatgttcaaccacgtggctgtggcagcccgctatgctcaacagaaacaccgcacccggagggtccttatcgtagattgggatgtgcaccacggtcaaggaacacagttcaccttcgaccaggaccccagtgtcctctatttctccatccaccgctacgagcagggtaggttctggccccacctgaaggcctctaactggtccaccacaggtttcggccaaggccaaggatataccatcaatgtgccttggaaccaggtggggatgcgggatgctgactacattgctgctttcctgcacgtcctgctgccagtcgccctcgagctccagcctcagctggtcctggtggccgctggatttgatgccctgcaaggggaccccaagggcgagatggccgccactccggcagggttcgcccagctaacccacctgctcatgggtctggcaggaggcaagctgatcctgtctctggagggtggctacaacctccgcgccctggctgaaggcgtcagtgcttcgctccacacccttctgggagacccttgccccatgccggagtcacctggtgccccctgccggagcgcccaggcttcagtttcctgtgctctggaagcccttgagcccttctgggaggttcttgtgagatcaactgagaccgtggagagggacaacatggaggaggacaatgtagaggagagcgaggaggaaggaccctgggagccccctgtgctcccaatcctgacatggccagtgctacagtctcgcacagggctggtctatgaccaaaatatgatgaatcactgcaacttgtgggacagccaccaccctgaggtaccccagcgcatcttgcggatcatgtgccgtctggaggagctgggccttgccgggcgctgcctcaccctgacaccgcgccctgccacagaggctgagctgctcacctgtcacagtgctgagtacgtgggtcatctccgggccacagagaaaatgaaaacccgggagctgcaccgtgagagttccaactttgactccatctatatctgccccagtaccttcgcctgtgcacagcttgccactggcgctgcctgccgcctggtggaggctgtgctctcaggagaggtcctgaatggtgctgctgtggtgcgtcccccaggacaccacgcagagcaggatgcagcttgcggtttttgctttttcaactctgtggctgtggctgctcgccatgcccagactatcagtgggcatgccctacggatcctgattgtggattgggatgtccaccacggtaatggaactcagcacatgtttgaggatgaccccagtgtgctatatgtgtccctgcaccgctatgatcatggcaccttcttccccatgggggatgagggtgccagcagccagatcggccgggccgcgggcacaggcttcaccgtcaacgtggcatggaacgggccccgcatgggtgatgctgactacctagctgcctggcatcgcctggtgcttcccattgcctacgagtttaacccagaactggtgctggtctcagctggctttgatgctgcacggggggatccgctggggggctgccaggtgtcacctgagggttatgcccacctcacccacctgctgatgggccttgccagtggccgcattatccttatcctagagggtggctataacctgacatccatctcagagtccatggctgcctgcactcgctccctccttggagacccaccacccctgctgaccctgccacggcccccactatcaggggccctggcctcaatcactgagaccatccaagtccatcgcagatactggcgcagcttacgggtcatgaaggcagaagacagagaaggaccctccagttctaagttggtcaccaagaaggcaccccaaccagccaaacctaggttagctgagcggatgaccacacgagaaaagaaggttctggaagcaggcatggggaaagtcacctcggcatcatttggggaagagtccactccaggccagactaactcagagacagctgtggtggccctcactcaggaccagccctcagaggcagccacagggggagccactctggcccagaccatttctgaggcagccattgggggagccatgctgggccagaccacctcagaggaggctgtcgggggagccactccggaccagaccacctcagaggagactgtgggaggagccattctggaccagaccacctcagaggatgctgttgggggagccacgctgggccagactacctcagaggaggctgtaggaggagctacactggcccagaccatctcggaggcagccatggagggagccacactggaccagactacgtcagaggaggctccagggggcaccgagctgatccaaactcctctagcctcgagcacagaccaccagacccccccaacctcacctgtgcagggaactacaccccagatatctcccagtacactgattgggagtctcaggaccttggagctaggcagcgaacctcagggggcctcagaatctcaggccccaggagaggagaacctaccaggagaggcagctggaggtcaggacatggctgattcgatgctgacgcagggatctaggggcctcactgatcaggccatattttatgctgtgacaccactgccctggtgtccccattcggtggcagtatgccccatacctgcagcaggcctagacgtgacccaaccttgtggggactgtggaacaatccaagagaactgggtgtgtctctcttgctatcaggtctaccgtggtcgttacatcaatggccacatgctccaacaccatggaaattctggacacccgctggtcctcagccacatcgacctgtcagcctggcgttactactgtcaggcctatgtccaccaccaggctctcctagatgtgaagaacatcgcccaccagaacaagtttggggaggatatgccccacccacactaagccccagaatacggtccctcttcaccttctgaggcccacgatagaccagttccagcctgttccaggctgtaccttggatgaggggtagcctcccactgcatcccatcctgaatatcctttgcaactccccaagagtgcttatttaagtgttaatacttttaagagaactgcgacgattaattgtggatctccccctgcccatcgcccgcttgaggggcaccactactccagcccagaaggaaaggggggcagctcagtggccccaagagggagccgatatcatgaggataacattggcgggaggggagttaactggcaggcatggcaaggttgcatatgtaataaagtacaagctgtt

SEQ ID NO: 13 - protein, 855 aa, Human
MDLRVGQRPPVEPPPEPTLLALQRPQRLHHHLFLAGLQQQRSVEPMRLSMDTPMPELQVGPQEQELRQLLHKDKSKRSAVASSVVKQKLAEVILKKQQAALERTVHPNSPGIPYRTLEPIETEGATRSMLSSFLPPVPSIPSDPPEHFPLRKTVSEPNLKLRYKPKKSLERRKNPLLRKESAPPSLRRRPAETLGDSSPSSSSTPASGCSSPNDSEHGPNPILGDSDRRTHPTLGPRGPILGSPHTPLFLPHGLEPEAGGCLPSRLQPILLLDPSGSHAPLLTVPGLGPLPFHFAQSIMTTERLSGSGLHWPLSRTRSEPLPPSATAPPPPGPMQPRLEQLKTHVQVIKRSAKPSEKPRLRQIPSAEDLETDGGGPGQVVDDGLEHRELGHGQPEARGPAPLQQHPQVIIWEQQRLAGRLPRGSTGDCVILPLAQGGHRPLSRAQSSPAAPASISAPEPASQARVLSSSETPARTLPFLTGLIYDSVMLKHQCSCGDNSRHPEHAGRIQSIWSRLQERGLRSQCECLRGRKASIEELQSVHSERHVLLYGTNPLSRLKLDNGKLAGIIAQRMFEMLPCGGVGVDTDTIWNELHSSNAARWAAGSVTDLAFKVASRELKNGFAVVRPPGHHADHSTAMGFCFFNSVAIACRQLQQQSKASKASKILIVDWDVHHGNGTQQTFYQDPSVLYISLHRHDDGNFFPGSGAVDEVGAGSGEGFNVNVAWAGGLDPPMGDPEYLAAFRIVVMPIAREFSPDLVLVSAGFDAAEGHPAPLGGYHVSAKCFGYMTQQLMNLAGGAVVLALEGGHDLTAICDASEACVAALLGNRVDPLSEEGWKQKPQPQCHPLSGGRDPGAQ

SEQ ID NO: 14 - DNA, 3131 bp, Human
ataatacctaccttgcaggaccacgacaggattaagtgaggaaaaacccccatgagagtgttttgccattgtcaagtgagcctgagggaggctgaggggggatcaggctgtatcatgcccccgaggacaaactttccagtttaccctgctccctctctctgtccctaggctgccccaggccctgcgcagacacaccaggccctcagccgcagcccatggacctgcgggtgggccagcggcccccagtggagcccccaccagagcccacattgctggccctgcagcgtccccagcgcctgcaccaccacctcttcctagcaggcctgcagcagcagcgctcggtggagcccatgaggctctccatggacacgccgacgcccgagttgcaggtgggaccccaggaacaagagctgcggcagcttctccacaaggacaagagcaagcgaagtgctgtagccagcagcgtggtcaagcagaagctagcggaggtgattctgaaaaaacagcaggcggccctagaaagaacagtccatcccaacagccccggcattccctacagaaccccggagcccctggagacggaaggagccacccgctccatgctcagcagccttccgcctcctgctcccagcccgcccagtgaccccccagagcactcccctctgcgcaagacagtctctgagcccaacctgaagctgcgccataagcccaagaagtccccggagcggaggaagaatccactgctccgaaaggagagtgcgccccccagcccccggcggcggcccgcagagaccctcggagactcctccccaagtagtagcagcacgcccgcatcagggtgcagtccccccaatgacagcgagcacggccccaatcccatcctgggcgacagtgaccgcaggacccatccgactctgggcccccgggggccaatcctggggagcccccacactcccctcttcctgccccatggcttggagcccgaggctgggggcaccttgccctcccgcctgcagcccattcctctcctggacccctcaggctctcatgccccgctgctgactgtgcccgggcttgggcccttgcccttccactttgcccagtccttaatgaccaccgagcggctctctgggtcaggcctccactggccactgagccggactcgctcagagcccctgccccccagtgccaccgctcccccaccgccgggccccatgcagccccgcctggagcagctcaaaactcacgtccaggtgatcaagaggtcagccaagccgagtgagaagccccggctgcggcagataccctcggctgaagacctggagacagatggcgggggaccgggccaggtggtggacgacggcccggagcacagggagctgggccatgggcagcccgaggccagaggccccgctcctctccagcagcaccctcaggtgttgctctgggaacagcagcgactggctgggcggctcccccggggcagcaccggggacactgtgctgcttcctctggcccagggtgggcaccggcctctgtcccgggctcagtcttccccagccgcacctgcctcactgtcagccccagagcctgccagccaggcccgagtcctctccagctcagagacccctgccaggaccctgcccttcaccacagggctgatctatgactcggtcatgctgaagcaccagtgctcctgcggtgacaacagcaggcacccggagcacgccggccgcatccagagcatctggtcccggctgcaggagcgggggcctcggagccagtgtgagtgtctccgaggccggaaggcctccctggaagagctgcagtcggtccactctgagcggcacgtgctcctctacggcaccaacccgctcagccgcctcaaactggacaacgggaagctggcagggctcctggcacagcggatgtttgagatgctgccctgtggtggggttggggtggacactgacaccatctggaatgagcttcattcctccaatgcagcccgctgggccgctggcagtgtcactgacctcgccttcaaagtggcttctcgtgagctaaagaatggtttcgctgtggtgcggcccccaggacaccatgcagatcattcaacagccatgggcttctgcttcttcaactcagtggccatcgcctgccggcagctgcaacagcagagcaaggccagcaaggccagcaagatcctcattgtagactgggacgtgcaccatggcaacggcacccagcaaaccttctaccaagaccccagtgtgctctacatctccctgcatcgccatgacgacggcaacttcttcccggggagtggggctgtggatgaggtaggggctggcagcggtgagggcttcaatgtcaatgtggcctgggctggaggtctggacccccccatgggggatcctgagtacctggctgctttcaggatagtcgtgacgcccatcgcccgagagttctctccagacctagtcctggtgtctgccggatttgatgctgctgagggtcacccggccccactgggtggctaccatgtttctgccaaatgttttggatacatgacgcagcaactgatgaacctggcaggaggcgcagtggtgctggccttggagggtggccatgacctcacagccatctgtgacgcctctgaggcctgtgtggctgctcttctgggtaacagggtggatcccctttcagaagaaggctggaaacagaaaccccaacctcaatgccactcgctctctggaggccgtgatccgggtgcacagtaaatactggggctgcatgcagcgcctggcctcctgtccagactcctgggtgcctagagtgccaggggctgacaaagaagaagtggaggcagtgaccgcactggcgtccctctctgtgggcatcctggctgaagataggccctcggagcagctggtggaggaggaagaacctatgaatctctaaggctctggaaccatctgcccgcccaccatgcccttgggacctggttctcttctaacccctggcaatagcccccattcctgggtctttagagatcctgtgggcaagtagttggaaccagagaacagcctgcctgctttgacagttatcccagggagcgtgagaaaatc

SEQ ID NO: 15 - protein, 377 aa, Human
MEEPEEPADSGQSLVPVYIYSPEYVSMCDSLAKIPKRASMVHSLIEAYALHKQMRIVKPKVASMEEMATFHTDAYLQHLQKVSQEGDDDHPDSIEYGLGYDCPATEGIFDYAAAIGGATITAAQCLIDGMCKVAINWSGGWHHAKKDEASGFCYLNDAVLGILRLRRKFERILYVDLDLHHGDGVEDAFSFTSKVMTVSLHKFSPGFFPGTGDVSDVGLGKGRYYSVNVPIQDGIQDEKYYQICESVLKEVYQAFNPKAVVLQLGADTIAGDPMCSFNMTPVGIGKCLKYILQWQLATLILGGGGYNLANTARCWTYLTGVILGKTLSSEIPDBEFFTAYGPDYVLEITPSCRPDRNEPHRIQQILNYIKGNLKHVV

SEQ ID NO: 16 - DNA, 1654 bp, Human
  misc_feature (1590)..(1641): Nucleotides 1590, 1592, 1600, 1607, 1611, 1630 and 1641 are “n” wherein “n” = any nucleotide.
gaaattcggcacgagctcgtgccgaattcggcacgagaacggttttaagcggaagatggaggagccggaggaaccggcggacagtgggcagtcgctggtcccggtttatatctatagtcccgagtatgtcagtatgtgtgactccctggccaagatccccaaacgggccagtatggtgcattctttgattgaagcatatgcactgcataagcaaatgaggatagttaagcctaaagtggcctccatggaggagatggccaccttccacactgatgcttatctgcagcatctccagaaggtcagccaagagggcgatgatgatcatccggactccatagaatatgggctaggttatgactgcccagccactgaagggatatttgactatgcagcagctataggaggggctacgatcacagctgcccaatgcctgattgacggaatgtgcaaagtagcaatcaactggtctggagggtggcatcatgcaaagaaagatgaagcatctggttttcgttatctcaatgatgctgtcctgggaatattacgattgcgacggaaatttgagcgtattccctacgtggattcggatctgcaccatggagatggtgtagaagacgcattcagtttcacctccaaagtcatgaccgtgtccctgcacaaattctccccaggatttttcccaggaacaggtgacgtgtccgacgttggcctagggaagggacggtactacagtgtaaatgtgcccatccaggatggcatacaagatgaaaaatattaccagatctgcgaaagtgtactaaaggaagtataccaagcctttaatcccaaagcagtggtcttacagctgggagccgacacaatagctggggatcccatgtgctcctttaacatgactccagtgggaattggcaagtgtctcaagtacatccctcaatggcagttggcaacactcatttcgggaggaggaggctataaccttgccaacacggctcgatgctggacatacttgaccggggtcatcctagggaaaacactatcctctgagatcccagatcatgagtttttcacagcatatggtcctgattatgtgctggaaatcacgccaagctgccggccagaccgcaatgagccccaccgaatccaacaaatcctcaactacatcaaagggaatctgaagcatgtggtctagttgacagaaagagatcaggtttccagagctgaggagtggtgcctataatgaagacagcgtgtttatgcaagcagtttgrggaatttgtgactgcagggaaaatttgaaagaaattacttcctgaaaatttccaaggggcatcaagtggcagctggcttcctggggtgaagaggcaggcaccccagagtcctcaactggacctaggggaagaaggagatarcccacatttaaagttcttatttaaaaaaacacacacacacaaatgaaatttttaatctttgaaaattatttttaagcgaattggggaggggagtattttaatcatcttaaatgaaacagatcagaagctggatgagagcagtcaccagtttgtagggcaggaggcagctgacaggcagggntngggcctcnggaccanccangtggagccctgggagaganggtactgatcngcagactgggagg

SEQ ID NO: 17 - DNA, 20 bp, Human
gaaacgtgagggactcagca

SEQ ID NO: 18 - DNA, 20 bp, Human
ggaagccagagctggagagg

SEQ ID NO: 19 - DNA, 20 bp, Human
gttaggtgaggcactgagga

SEQ ID NO: 20 - DNA, 20 bp, Human
gctgagctgttctgatttgg

SEQ ID NO: 21 - DNA, 20 bp, Human
cgtgagcacttctcatttcc

SEQ ID NO: 22 - DNA, 20 bp, Human
cgctttccttgtcattgaca

SEQ ID NO: 23 - DNA, 20 bp, Human
gcctttcctactcattgtgt

SEQ ID NO: 24 - DNA, 20 bp, Human
gctgcctgccgtgcccaccc

SEQ ID NO: 25 - DNA, 20 bp, Human
cgtgcctgcgctgcccacgg

SEQ ID NO: 26 - DNA, 20 bp, Human
tacagtccatgcaacctcca

SEQ ID NO: 27 - DNA, 20 bp, Human
atcagtccaaccaacctcgt

SEQ ID NO: 28 - DNA, 20 bp, Human
cttcggtctcacctgcttgg

SEQ ID NO: 29 - DNA, 20 bp, Human
caggctggaatgagctacag

SEQ ID NO: 30 - DNA, 20 bp, Human
gacgctgcaatcaggtagac

SEQ ID NO: 31 - DNA, 20 bp, Human
cttcagccaggatgcccaca

SEQ ID NO: 32 - DNA, 20 bp, Human
ctccggctcctccatcttcc

SEQ ID NO: 33 - DNA, 20 bp, Human
agccagctgccacttgatgc

(19) United States
(12) Patent Application Publication
(10) Pub. No.: US 2004/0266718 A1
(43) Pub. Date: Dec. 30, 2004

(54) Inhibition of specific histone deacetylase isoforms

(76) Inventors: Zuomei Li, Kirkland (CA); Claire Bonfils, Montreal (CA); Jeffrey Besterman, Baie D'Urfe (CA)

Correspondence Address: KEOWN & ASSOCIATES, SUITE 1200, 500 WEST CUMMING PARK, WOBURN, MA, 01801, US

(21) Appl. No.: 10/870,587

(22) Filed: Jun. 17, 2004

Related U.S. Application Data
(62) Division of application No. 09/817,913, filed on Aug. 6, 2001.

(51) Int. Cl. A61K 48/00

(52) U.S. Cl. 514/44

(57) ABSTRACT

This invention relates to the inhibition of histone deacetylase expression and enzymatic activity. The invention provides methods and reagents for inhibiting specific histone deacetylase (HDAC) isoforms by inhibiting expression at the nucleic acid level or enzymatic activity at the protein level.

### CROSS-REFERENCE TO RELATED APPLICATION

This application claims the benefit of U.S. Provisional Application No. 60/192,157, filed Mar. 24, 2000.

### BACKGROUND OF THE INVENTION

1. Field of the Invention

This invention relates to the fields of inhibition of histone deacetylase expression and enzymatic activity.

2. Summary of the Related Art

In eukaryotic cells, nuclear DNA associates with histones to form a compact complex called chromatin. The histones constitute a family of basic proteins which are generally highly conserved across eukaryotic species. The core histones, termed H2A, H2B, H3, and H4, associate to form a protein core. DNA winds around this protein core, with the basic amino acids of the histones interacting with the negatively charged phosphate groups of the DNA. Approximately 146 base pairs of DNA wrap around a histone core to make up a nucleosome particle, the repeating structural motif of chromatin.

Csordas, Biochem. J., 286: 23-38 (1990) teaches that histones are subject to posttranslational acetylation of the epsilon-amino groups of N-terminal lysine residues, a reaction that is catalyzed by histone acetyl transferase (HAT1). Acetylation neutralizes the positive charge of the lysine side chain, and is thought to impact chromatin structure. Indeed, Taunton et al., Science, 272: 408-411 (1996), teaches that access of transcription factors to chromatin templates is enhanced by histone hyperacetylation. Taunton et al. further teaches that an enrichment in underacetylated histone H4 has been found in transcriptionally silent regions of the genome.

Recently, there has been interest in the role of histone deacetylase (HDAC) in gene expression. Sanches Del Pino et al., Biochem. J. 303: 723-729 (1994) discloses a partially purified yeast HDAC activity. Taunton et al., (Supra): discloses a human HDAC that is related to a yeast transcriptional regulator and suggests that this protein may be a key regulator of eukaryotic transcription.

Known inhibitors of mammalian HDAC have been used to probe the role of HDAC in gene regulation. Yoshida et al., J. Biol. Chem 265: 17174-17179 (1990) discloses that (R)-Trichostatin A (TSA) is a potent inhibitor of mammalian HDAC. Yoshida et al, Cancer Research 47: 3688-3691 (1987) discloses that TSA is a potent inducer of differentiation in murine erythroleukemia cells.

More recently, it has been discovered that the HDAC activity is actually provided by a set of discrete HDAC enzyme isoforms. Grozinger et al., Proc. Natl. Acad. Sci. USA, 96: 4868-4873 (1999), teaches that HDACs may be divided into two classes, the first represented by yeast Rpd3-like proteins, and the second represented by yeast Hda1-like proteins. Grozinger et al. also teaches that the human HDAC1, HDAC2, and HDAC3 proteins are members of the first class of HDACs, and discloses new proteins, named HDAC4, HDAC5, and HDAC6, which are members of the second class of HDACs. Kao et al., Gene & Development 14: 55-66 (2000), discloses an additional member of this second class, called HDAC-7. More recently, Hu, E. et al. J. Bio. Chem. 275:15254-13264 (2000) disclosed the newest member of the first class of histone deacetylases, HDAC-8. It has been unclear what roles these individual HDAC enzymes play.

The known inhibitors of histone deacetylase are all small molecules that inhibit histone deacetylase activity at the protein level. Moreover, all of the known histone deacetylase inhibitors are non-specific for a particular histone deacetylase isoform, and more or less inhibit all members of both the histone deacetylase families equally.

Therefore, there remains a need to develop reagents for inhibiting specific histone deacetylase isoforms. There is also a need for the development of methods for using these reagents to identify and inhibit specific histone deacetylase isoforms involved in tumorigenesis.

### BRIEF SUMMARY OF THE INVENTION

The invention provides methods and reagents for inhibiting specific histone deacetylase (HDAC) isoforms by inhibiting expression at the nucleic acid level or enzymatic activity at the protein level. The invention allows the identification of and specific inhibition of specific histone deacetylase isoforms involved in tumorigenesis and thus provides a treatment for cancer. The invention further allows identification of and specific inhibition of specific HDAC isoforms involved in cell proliferation and/or differentiation and thus provides a treatment for cell proliferative and/or differentiation disorders.

The inventors have discovered new agents that inhibit specific HDAC isoforms. Accordingly, in a first aspect, the invention provides agents that inhibit one or more specific histone deacetylase isoforms but less than all histone deacetylase isoforms. Such specific HDAC isoforms include without limitation, HDAC-1, HDAC-2, HDAC-3, HDAC-4, HDAC-5, HDAC-6, HDAC-7 and HDAC-8. Non-limiting examples of the new agents include antisense oligonucleotides (oligos) and small molecule inhibitors specific for one or more HDAC isoforms but less than all HDAC isoforms.

The present inventors have surprisingly discovered that specific inhibition of HDAC-1 reverses the tumorigenic state of a transformed cell. The inventors have also surprisingly discovered that the inhibition of the HDAC-4 isoform dramatically induces growth and apoptosis arrest in cancerous cells. Thus, in certain embodiments of this aspect of the invention, the histone deacetylase isoform that is inhibited is HDAC-1 and/or HDAC-4.

In certain preferred embodiments, the agent that inhibits the specific HDAC isoform is an oligonucleotide that inhibits expression of a nucleic acid molecule encoding that histone deacetylase isoform. The nucleic acid molecule may be genomic DNA (e.g., a gene), cDNA, or RNA. In some embodiments, the oligonucleotide inhibits transcription of mRNA encoding the HDAC isoform. In other embodiments, the oligonucleotide inhibits translation of the histone deacetylase isoform. In certain embodiments the oligonucleotide causes the degradation of the nucleic acid molecule. Particularly preferred embodiments include antisense oligonucleotides directed to HDAC-1 and/or HDAC-4.

In yet other embodiments of the first aspect, the agent that inhibits a specific HDAC isoform is a small molecule inhibitor that inhibits the activity of one or more specific histone deacetylase isoforms but less than all histone deacetylase isoforms.

In a second aspect, the invention provides a method for inhibiting one or more, but less than all, histone deacetylase isoforms in a cell, comprising contacting the cell with an agent of the first aspect of the invention. In other preferred embodiments, the agent is an antisense oligonucleotide. In certain preferred embodiments, the agent is a small molecule inhibitor. In other certain preferred embodiments of the second aspect of the invention, cell proliferation is inhibited in the contacted cell. In preferred embodiments, the cell is a neoplastic cell which may be in an animal, including a human, and which may be in a neoplastic growth. In certain preferred embodiments, the method of the second aspect of the invention further comprises contacting the cell with a histone deacetylase small molecule inhibitor that interacts with and reduces the enzymatic activity of one or more specific histone deacetylase isoforms. In still yet other preferred embodiments of the second aspect of the invention, the method comprises an agent of the first aspect of the invention which is a combination of one or more antisense oligonucleotides and/or one or more small molecule inhibitors of the first aspect of the invention. In certain preferred embodiments, the histone deacetylase isoform is HDAC-1, HDAC-2, HDAC-3, HDAC-4, HDAC-5, HDAC-6, HDAC-7, or HDAC-8. In other certain preferred embodiments, the histone deacetylase isoform is HDAC-1 and/or HDAC-4. In some embodiments, the histone deacetylase small molecule inhibitor is operably associated with the antisense oligonucleotide.

In a third aspect, the invention provides a method for inhibiting neoplastic cell proliferation in an animal comprising administering to an animal having at least one neoplastic cell present in its body a therapeutically effective amount of an agent of the first aspect of the invention. In certain preferred embodiments, the agent is an antisense oligonucleotide which is combined with a pharmaceutically acceptable carrier and administered for a therapeutically effective period of time. In certain preferred embodiments, the agent is a small molecule inhibitor which is combined with a pharmaceutically acceptable carrier and administered for a therapeutically effective period of time. In certain preferred embodiments of the this aspect of the invention, cell proliferation is inhibited in the contacted cell. In preferred embodiments, the cell is a neoplastic cell which may be in an animal, including a human, and which may be in a neoplastic growth. In other certain embodiments, the agent is a small molecule inhibitor of the first aspect of the invention which is combined with a pharmaceutically acceptable carrier and administered for a therapeutically effective period of time. In still yet other preferred embodiments of the third aspect of the invention, the method comprises an agent of the first aspect of the invention which is a combination of one or more antisense oligonucleotides and/or one or more small molecule inhibitors of the first aspect of the invention. In certain preferred embodiments, the histone deacetylase isoform is HDAC-1, HDAC-2, HDAC-3, HDAC-4, HDAC-5, HDAC-6, HDAC-7, or HDAC-8. In other certain preferred embodiments, the histone deacetylase isoform is HDAC-1 and/or HDAC-4.

In a fourth aspect, the invention provides a method for identifying a specific histone deacetylase isoform that is required for induction of cell proliferation comprising contacting a cell with an agent of the first aspect of the invention. In certain preferred embodiments, the agent is an antisense oligonucleotide that inhibits the expression of a histone deacetylase isoform, wherein the antisense oligonucleotide is specific for a particular HDAC isoform, and thus inhibition of cell proliferation in the contacted cell identifies the histone deacetylase isoform as a histone deacetylase isoform that is required for induction of cell proliferation. In other certain embodiments, the agent is a small molecule inhibitor that inhibits the activity of a histone deacetylase isoform, wherein the small molecule inhibitor is specific for a particular HDAC isoform, and thus inhibition of cell proliferation in the contacted cell identifies the histone deacetylase isoform as a histone deacetylase isoform that is required for induction of cell proliferation. In certain preferred embodiments, the cell is a neoplastic cell, and the induction of cell proliferation is tumorigenesis. In still yet other preferred embodiments of the fourth aspect of the invention, the method comprises an agent of the first aspect of the invention which is a combination of one or more antisense oligonucleotides and/or one or more small molecule inhibitors of the first aspect of the invention. In certain preferred embodiments, the histone deacetylase isoform is HDAC-1, HDAC-2, HDAC-3, HDAC-4, HDAC-5, HDAC-6, HDAC-7, or HDAC-8. In other certain preferred embodiments, the histone deacetylase isoform is HDAC-1 and/or HDAC-4.

In an fifth aspect, the invention provides a method for identifying a histone deacetylase isoform that is involved in induction of cell differentiation, comprising contacting a cell with an agent that inhibits the expression of a histone deacetylase isoform, wherein induction of differentiation in the contacted cell identifies the histone deacetylase isoform as a histone deacetylase isoform that is involved in induction of cell differentiation. In certain preferred embodiments, the agent is an antisense oligonucleotide of the first aspect of the invention. In other certain preferred embodiments, the agent is an small molecule inhibitor of the first aspect of the invention. In still other certain embodiments, the cell is a neoplastic cell. In still yet other preferred embodiments of the fifth aspect of the invention, the method comprises an agent of the first aspect of the invention which is a combination of one or more antisense oligonucleotides and/or one or more small molecule inhibitors of the first aspect of the invention. In certain preferred embodiments, the histone deacetylase isoform is HDAC-1, HDAC-2, HDAC-3, HDAC-4, HDAC-5, HDAC-6, HDAC-7, or HDAC-8. In other certain preferred embodiments, the histone deacetylase isoform is HDAC-1 and/or HDAC-4.

In a sixth aspect, the invention provides a method for inhibiting neoplastic cell growth in an animal comprising administering to an animal having at least one neoplastic cell present in its body a therapeutically effective amount of an agent of the first aspect of the invention. In certain embodiments thereof, the agent is an antisense oligonucleotide, which is combined with a pharmaceutically acceptable carrier and administered for a therapeutically effective period of time.

In an seventh aspect, the invention provides a method for identifying a histone deacetylase isoform that is involved in induction of cell differentiation, comprising contacting a cell with an antisense oligonucleotide that inhibits the expression of a histone deacetylase isoform, wherein induction of differentiation in the contacted cell identifies the histone deacetylase isoform as a histone deacetylase isoform that is involved in induction of cell differentiation. Preferably, the cell is a neoplastic cell. In certain preferred embodiments, the histone deacetylase isoform is HDAC-1, HDAC-2, HDAC-3, HDAC-4, HDAC-5, HDAC-6, HDAC-7, or HDAC-8. In other certain preferred embodiments, the histone deacetylase isoform is HDAC-1 and/or HDAC-4.

In an eighth aspect, the invention provides a method for inhibiting cell proliferation in a cell comprising contacting a cell with at least two reagents selected from the group consisting of an antisense oligonucleotide from the first aspect of the invention that inhibits expression of a specific histone deacetylase isoform, a small molecule inhibitor from the first aspect of the invention that inhibits a specific histone deacetylase isoform, an antisense oligonucleotide that inhibits a DNA methyltransferase, and a small molecule that inhibits a DNA methyltransferase. In one embodiment, the inhibition of cell growth of the contacted cell is greater than the inhibition of cell growth of a cell contacted with only one of the reagents. In certain embodiments, each of the reagents selected from the group is substantially pure. In preferred embodiments, the cell is a neoplastic cell. In yet additional preferred embodiments, the reagents selected from the group are operably associated. In certain preferred embodiments, the histone deacetylase isoform is HDAC-1, HDAC-2, HDAC-3, HDAC-4, HDAC-5, HDAC-6, HDAC-7, or HDAC-8. In other certain preferred embodiments, the histone deacetylase isoform is HDAC-1 and/or HDAC-4.

In a ninth aspect, the invention provides a method for modulating cell proliferation or differentiation, comprising contacting a cell with an agent of the first aspect of the invention, wherein one or more, but less than all, HDAC isoforms are inhibited, which results in a modulation of proliferation or differentiation. In certain embodiments, the agent is an antisense oligonucleotide of the first aspect of the invention. In other certain preferred embodiments, the agent is a small molecule inhibitor of the first aspect of the invention. In preferred embodiments, the cell proliferation is neoplasia. In still yet other preferred embodiments of the this aspect of the invention, the method comprises an agent of the first aspect of the invention which is a combination of one or more antisense oligonucleotides and/or one or more small molecule inhibitors of the first aspect-of the invention. In certain preferred embodiments, the histone deacetylase isoform is HDAC-1, HDAC-2, HDAC-3, HDAC-4, HDAC-5, HDAC-6, HDAC-7, or HDAC-8. In other certain preferred embodiments, the histone deacetylase isoform is HDAC-1 and/or HDAC-4.

### BRIEF DESCRIPTION OF THE DRAWINGS

FIG. 1A is a schematic diagram providing the amino acid sequence of HDAC-1, as provided in GenBank Accession No. AAC50475 (SEQ ID NO:1).

FIG. 1B is a schematic diagram providing the nucleic acid sequence of HDAC-1, as provided in GenBank Accession No. U50079 (SEQ ID NO:2).

FIG. 2A is a schematic diagram providing the amino acid sequence of HDAC-2, as provided in GenBank Accession No. AAC50814 (SEQ ID NO:3).

FIG. 2B is a schematic diagram providing the nucleic acid sequence of HDAC-2, as provided in GenBank Accession No. U31814 (SEQ ID NO:4).

FIG. 3A is a schematic diagram providing the amino acid sequence of HDAC-3, as provided in GenBank Accession No. AAB88241 (SEQ ID NO:5).

FIG. 3B is a schematic diagram providing the nucleic acid sequence of HDAC-3, as provided in GenBank Accession No. U75697 (SEQ ID NO:6).

FIG. 4A is a schematic diagram providing the amino acid sequence of HDAC-4, as provided in GenBank Accession No. BAA22957 (SEQ ID NO:7).

FIG. 4B is a schematic diagram providing the nucleic acid sequence of HDAC-4, as provided in GenBank Accession No. AB006626 (SEQ ID NO:8).

FIG. 5A is a schematic diagram providing the amino acid sequence of HDAC-5, as provided in GenBank Accession No. BAA25526 (SEQ ID NO:9).

FIG. 5B is a schematic diagram providing the nucleic acid sequence of HDAC-5 as provided in GenBank Accession No. AB011172 (SEQ ID NO:10).

FIG. 6A is a schematic diagram providing the amino acid sequence of human HDAC-6, as provided in GenBank Accession No. AAD29048 (SEQ ID NO:11).

FIG. 6B is a schematic diagram providing the nucleic acid sequence of human HDAC-6, as provided in GenBank Accession No. AJ011972 (SEQ ID NO:12).

FIG. 7A is a schematic diagram providing the amino acid sequence of human HDAC-7, as provided in GenBank Accession No. AAF63491.1 (SEQ ID NO:13).

FIG. 7B is a schematic diagram providing the nucleic acid sequence of human HDAC-7, as provided in GenBank Accession No. AF239243 (SEQ ID NO:14).

FIG. 8A is a schematic diagram providing the amino acid sequence of human HDAC-8, as provided in GenBank Accession No. AAF73076.1 (SEQ ID NO:15).

FIG. 8B is a schematic diagram providing the nucleic acid sequence of human HDAC-8, as provided in GenBank Accession No. AF230097 (SEQ ID NO:16).

FIG. 9A is a representation of a Northern blot demonstrating the effect of HDAC-1 AS1 antisense oligonucleotide on HDAC-1 mRNA expression in human A549 cells.

FIG. 9A is a representation of a Northern blot demonstrating the effect of HDAC-2 AS antisense oligonucleotide on HDAC-2 mRNA expression in human A549 cells.

FIG. 9C is a representation of a Northern blot demonstrating the effect of HDAC-6 AS antisense oligonucleotide on HDAC-6 mRNA expression in human A549 cells.

FIG. 9D is a representation of a Northern blot demonstrating the effect of HDAC-3 AS antisense oligonucleotide on HDAC-3 mRNA expression in human A549 cells.

FIG. 9E is a representation of a Northern blot demonstrating the effect of an HDAC-4 antisense oligonucleotide (AS1) on HDAC-4 mRNA expression in human A549 cells.

FIG. 9F is a representation of a Northern blot demonstrating the dose-dependent effect of an HDAC-4 antisense oligonucleotide (AS2) on HDAC-4 mRNA expression in human A549 cells.

FIG. 9G is a representation of a Northern blot demonstrating the effect of an HDAC-5 antisense oligonucleotide (AS) on HDAC-5 mRNA expression in human A549 cells.

FIG. 9H is a representation of a Northern blot demonstrating the effect of an HDAC-7 antisense oligonucleotide (AS) on HDAC-7 mRNA expression in human A549 cells.

FIG. 9I is a representation of a Northern blot demonstrating the dose-dependent effect of HDAC-8 antisense oligonucleotides (AS1 and AS2) on HDAC-8 mRNA expression in human A549 cells.

FIG. 10A is a representation of a Western blot demonstrating the effect of HDAC isotype-specific antisense oligos on HDAC isotype protein expression in human A549 cells.

FIG. 10B is a representation of a Western blot demonstrating the dose-dependent effect of the HDAC-1 isotype-specific antisense oligo (AS1 and AS2) on HDAC isotype protein expression in human A549 cells.

FIG. 10C is a representation of a Western blot demonstrating the effect of HDAC-4 isotype-specific antisense oligonucleotide (AS2) on HDAC isotype protein expression in human A549 cells.

FIG. 11A is a graphic representation demonstrating the apoptotic effect of HDAC isotype-specific antisense oligos on human A549 cancer cells.

FIG. 12A is a graphic representation demonstrating the effect of HDAC-1 AS1 and AS2 antisense oligonucleotides on the proliferation of human A549 cancer cells.

FIG. 12B is a graphic representation demonstrating the effect of HDAC-8 specific AS1 and AS2 antisense oligonucleotides on the proliferation of human A549 cancer cells.

FIG. 13 is a a graphic representation demonstrating the cell cycle blocking effect of HDAC specific antisense oligonucleotides on human A549 cancer cells.

FIG. 14 is a representation of an RNAse protection assay demonstrating the effect of HDAC isotype-specific antisense oligonucleotides on HDAC isotype mRNA expression in human A549 cells.

FIG. 15 is a representation of a Western blot demonstrating that treatment of human A549 cells with HDAC-4 AS1 antisense oligonucleotide induces the expression of the p21 protein.

FIG. 16 is a representation of a Western blot demonstrating that treatment of human A549 cells with HDAC-1 antisense oligonucleotides (AS1 and AS2) represses the expression of the cyclin B1 and cyclin A genes.

FIG. 17 shows plating data demonstrating the ability of antisense oligonucleotides complementary to HDAC-1 to inhibit growth in soft agar of A549 cells far more than can antisense oligonucleotides complementary to HDAC-2, HDAC-6 or mismatched controls.

FIG. 18 is a representation of a Western blot demonstrating that treatment of human A549 cells with the small molecule inhibitor Compound 3 (Table 2) induces the expression of the p21 protein and represses the expression of the cyclin B1 and cyclin A genes.

### DETAILED DESCRIPTION OF THE PREFERRED EMBODIMENTS

The invention provides methods and reagents for inhibiting specific histone deacetylase isoforms (HDAC) by inhibiting expression at the nucleic acid level or protein activity at the enzymatic level. The invention allows the identification of and specific inhibition of specific histone deacetylase isoforms involved in tumorigenesis and thus provides a treatment for cancer. The invention further allows identification of and specific inhibition of specific HDAC isoforms involved in cell proliferation and/or differentiation and thus provides a treatment for cell proliferative and/or differentiation disorders.

The patent and scientific literature referred to herein establishes knowledge that is available to those with skill in the art. The issued patents, applications, and references, including GenBank database sequences, that are cited herein are hereby incorporated by reference to the same extent as if each was specifically and individually indicated to be incorporated by reference.

In a first aspect, the invention provides agents that inhibit one or more histone deacetylase isoform, but less than all specific histone deacetylase isoforms. As used herein interchangeably, the terms “histone deacetylase”, “HDAC”, “histone deacetylase isoform”, “HDAC isoform” and similar terms are intended to refer to any one of a family of enzymes that remove acetyl groups from the epsilon-amino groups of lysine residues at the N-terminus of a histone. Unless otherwise indicated by context, the term “histone” is meant to refer to any histone protein, including H1, H2A, H2B, H3, and H4, from any species. Preferred histone deacetylase isoforms include class I and class II enzymes. Specific HDACs include without limitation, HDAC-1, HDAC-2, HDAC-3, HDAC-4, HDAC-5, HDAC-6, HDAC-7 and HDAC-8. By way of non-limiting example, useful agents that inhibit one or more histone deacetylase isoforms, but less than all specific histone deacetylase isoforms, include antisense oligonucleotides and small molecule inhibitors.

The present inventors have surprisingly discovered that specific inhibition of HDAC-1 reverses the tumorigenic state of a transformed cell. The inventors have also surprisingly discovered that the inhibition of the HDAC-4 isoform dramatically induces growth and apoptosis arrest in cancerous cells. Thus, in certain embodiments of this aspect of the invention, the histone deacetylase isoform that is inhibited is HDAC-1 and/or HDAC-4.

Preferred agents that inhibit HDAC-1 and/or HDAC-4 dramatically inhibit growth of human cancer cells, independent of p53 status. These agents significantly induce apoptosis in the cancer cells and cause dramatic growth arrest. They also can induce transcription of tumor suppressor genes, such as p21^{WAF1}, p57^{KIP2}, GADD153 and GADD45. Finally, they exhibit both in vitro and in vivo anti-tumor activity. Inhibitory agents that achieve one or more of these results are considered within the scope of this aspect of the invention. By way of non-limiting example, antisense oligonucleotides and/or small molecule inhibitors of HDAC-1 and/or HDAC-4 are useful for the invention.

In certain preferred embodiments, the agent that inhibits the specific HDAC isoform is an oligonucleotide that inhibits expression of a nucleic acid molecule encoding a specific histone deacetylase isoform. The nucleic acid molecule may be genomic DNA (e.g., a gene), cDNA, or RNA. In other embodiments, the oligonucleotide ultimately inhibits translation of the histone deacetylase. In certain embodiments the oligonucleotide causes the degradation of the nucleic acid molecule. Preferred antisense oligonucleotides have potent and specific antisense activity at nanomolar concentrations.

The antisense oligonucleotides according to the invention are complementary to a region of RNA or double-stranded DNA that encodes a portion of one or more histone deacetylase isoform (taking into account that homology between different isoforms may allow a single antisense oligonucleotide to be complementary to a portion of more than one isoform).

For purposes of the invention, the term “complementary” means having the ability to hybridize to a genomic region, a gene, or an RNA transcript thereof under physiological conditions. Such hybridization is ordinarily the result of base-specific hydrogen bonding between complementary strands, preferably to form Watson-Crick or Hoogsteen base pairs, although other modes of hydrogen bonding, as well as base stacking can lead to hybridization. As a practical matter, such hybridization can be inferred from the observation of specific gene expression inhibition, which may be at the level of transcription or translation (or both).

For purposes of the invention, the term “oligonucleotide” includes polymers of two or more deoxyribonucleosides, ribonucleosides, or 2′-O-substituted ribonucleoside residues, or any combination thereof. Preferably, such oligonucleotides have from about 8 to about 50 nucleoside residues, and most preferably from about 12 to about 30 nucleoside residues. The nucleoside residues may be coupled to each other by any of the numerous known internucleoside linkages. Such internucleoside linkages include without limitation phosphorothioate, phosphorodithioate, alkylphosphonate, alkylphosphonothioate, phosphotriester, phosphoramidate, siloxane, carbonate, carboxymethylester, acetamidate, carbamate, thioether, bridged phosphoramidate, bridged methylene phosphonate, bridged phosphorothioate, and sulfone internucleotide linkages. In certain preferred embodiments, these internucleoside linkages may be phosphodiester, phosphotriester, phosphorothioate, or phosphoramidate linkages, or combinations thereof. The term oligonucleotide also encompasses such polymers having chemically modified bases or sugars and/or having additional substituents, including without limitation lipophilic groups, intercalating agents, diamines, and adamantane. The term oligonucleotide also encompasses such polymers as PNA and LNA. For purposes of the invention the term “2′-O-substituted” means substitution of the 2′ position of the pentose moiety with an —O-lower alkyl group containing 1-6 saturated or unsaturated carbon atoms, or with an —O-aryl or allyl group having 2-6 carbon atoms, wherein such alkyl, aryl, or allyl group may be unsubstituted or may be substituted, e.g., with halo, hydroxy, trifluoromethyl, cyano, nitro, acyl, acyloxy, alkoxy, carboxyl, carbalkoxyl, or amino groups; or such 2′ substitution may be with a hydroxy group (to produce a ribonucleoside), an amino or a halo group, but not with a 2′-H group.

Particularly preferred antisense oligonucleotides utilized in this aspect of the invention include chimeric oligonucleotides and hybrid oligonucleotides.

For purposes of the invention, a “chimeric oligonucleotide” refers to an oligonucleotide having more than one type of internucleoside linkage. One preferred embodiment of such a chimeric oligonucleotide is a chimeric oligonucleotide comprising a phosphorothioate, phosphodiester or phosphorodithioate region, preferably comprising from about 2 to about 12 nucleotides, and an alkylphosphonate or alkylphosphonothioate region (see e.g., Pederson et al. U.S. Pat. Nos. 5,635,377 and 5,366,878). Preferably, such chimeric oligonucleotides contain at least three consecutive internucleoside linkages selected from phosphodiester and phosphorothioate linkages, or combinations thereof.

For purposes of the invention, a “hybrid oligonucleotide” refers to an oligonucleotide having more than one type of nucleoside. One preferred embodiment of such a hybrid oligonucleotide comprises a ribonucleotide or 2′-O-substituted ribonucleotide region, preferably comprising from about 2 to about 12 2′-O-substituted nucleotides, and a deoxyribonucleotide region. Preferably, such a hybrid oligonucleotide will contain at least three consecutive deoxyribonucleosides and will also contain ribonucleosides, 2′-O-substituted ribonucleosides, or combinations thereof (see e.g., Metelev and Agrawal, U.S. Pat. Nos. 5,652,355 and 5,652,356).

The exact nucleotide sequence and chemical structure of an antisense oligonucleotide utilized in the invention can be varied, so long as the oligonucleotide retains its ability to inhibit expression of a specific histone deacetylase isoform or inhibit one or more histone deacetylase isoforms, but less than all specific histone deacetylase isoforms. This is readily determined by testing whether the particular antisense oligonucleotide is active by quantitating the amount of mRNA encoding a specific histone deacetylase isoform, quantitating the amount of histone deacetylase isoform protein, quantitating the histone deacetylase isoform enzymatic activity, or quantitating the ability of the histone deacetylase isoform to inhibit cell growth in a an in vitro or in vivo cell growth assay, all of which are described in detail in this specification. The term “inhibit expression” and similar terms used herein are intended to encompass any one or more of these parameters.

Antisense oligonucleotides utilized in the invention may conveniently be synthesized on a suitable solid support using well-known chemical approaches, including H-phosphonate chemistry, phosphoramidite chemistry, or a combination of H-phosphonate chemistry and phosphoramidite chemistry (i.e., H-phosphonate chemistry for some cycles and phosphoramidite chemistry for other cycles). Suitable solid supports include any of the standard solid supports used for solid phase oligonucleotide synthesis, such as controlled-pore glass (CPG) (see, e.g., Pon, R. T., Methods in Molec. Biol. 20: 465-496, 1993).

Antisense oligonucleotides according to the invention are useful for a variety of purposes. For example, they can be used as “probes” of the physiological function of specific histone deacetylase isoforms by being used to inhibit the activity of specific histone deacetylase isoforms in an experimental cell culture or animal system and to evaluate the effect of inhibiting such specific histone deacetylase isoform activity. This is accomplished by administering to a cell or an animal an antisense oligonucleotide that inhibits one or more histone deacetylase isoform expression according to the invention and observing any phenotypic effects. In this use, the antisense oligonucleotides according to the invention is preferable to traditional “gene knockout” approaches because it is easier to use, and can be used to inhibit specific histone deacetylase isoform activity at selected stages of development or differentiation.

Preferred antisense oligonucleotides of the invention inhibit either the transcription of a nucleic acid molecule encoding the histone deacetylase isoform, and/or the translation of a nucleic acid molecule encoding the histone deacetylase isoform, and/or lead to the degradation of such nucleic acid. Histone deacetylase-encoding nucleic acids may be RNA or double stranded DNA regions and include, without limitation, intronic sequences, untranslated 5′ and 3′ regions, intron-exon boundaries as well as coding sequences from a histone deacetylase family member gene. For human sequences, see e.g., Yang et al., Proc. Natl. Acad. Sci. USA 93(23): 12845-12850, 1996; Furukawa et al., Cytogenet. Cell Genet. 73(1-2): 130-133, 1996; Yang et al., J. Biol. Chem. 272(44): 28001-28007, 1997; Betz et al., Genomics 52(2): 245-246, 1998; Taunton et al., Science 272(5260): 408-411, 1996; and Dangond et al., Biochem. Biophys. Res. Commun. 242(3): 648-652, 1998).

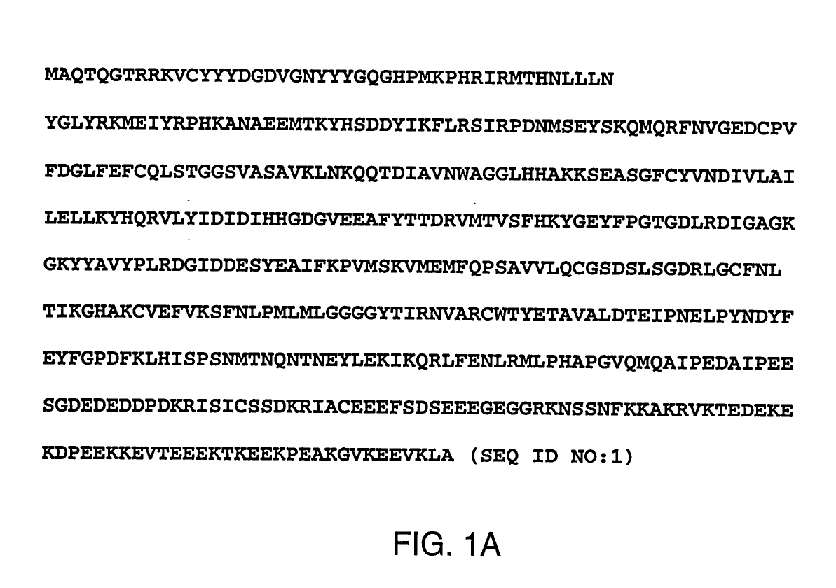

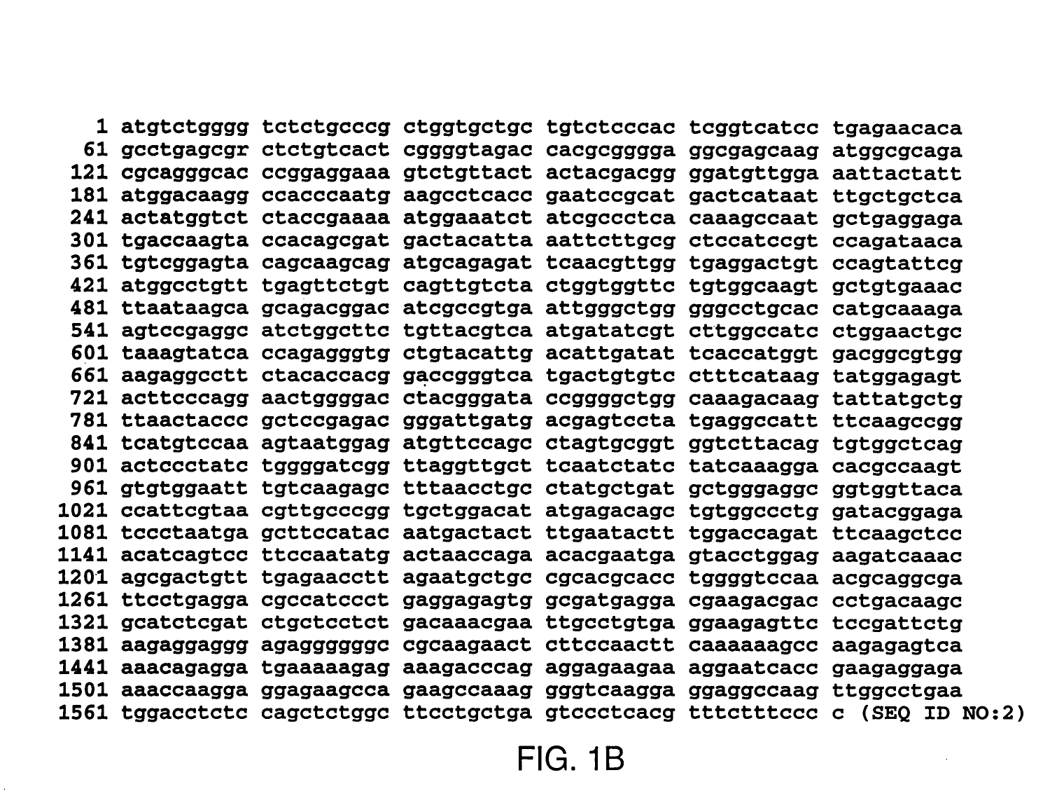

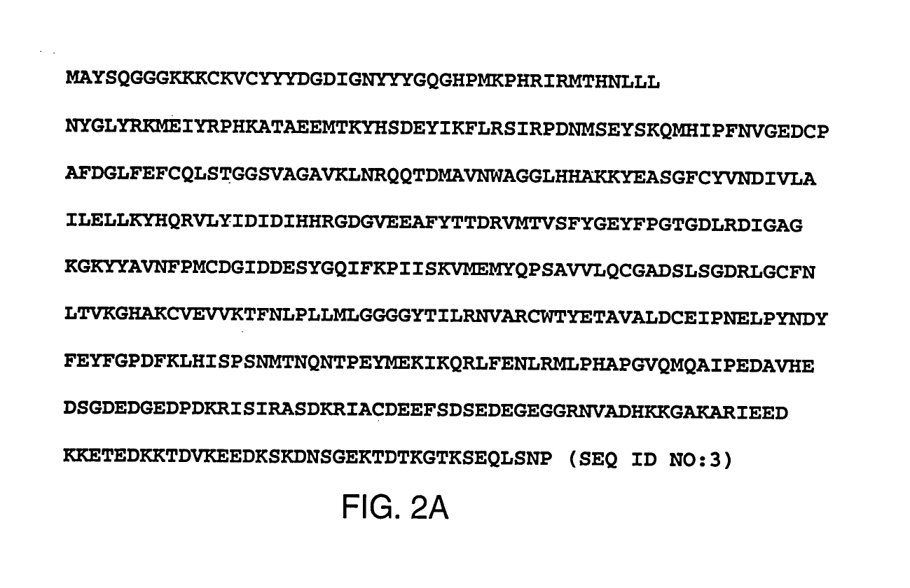

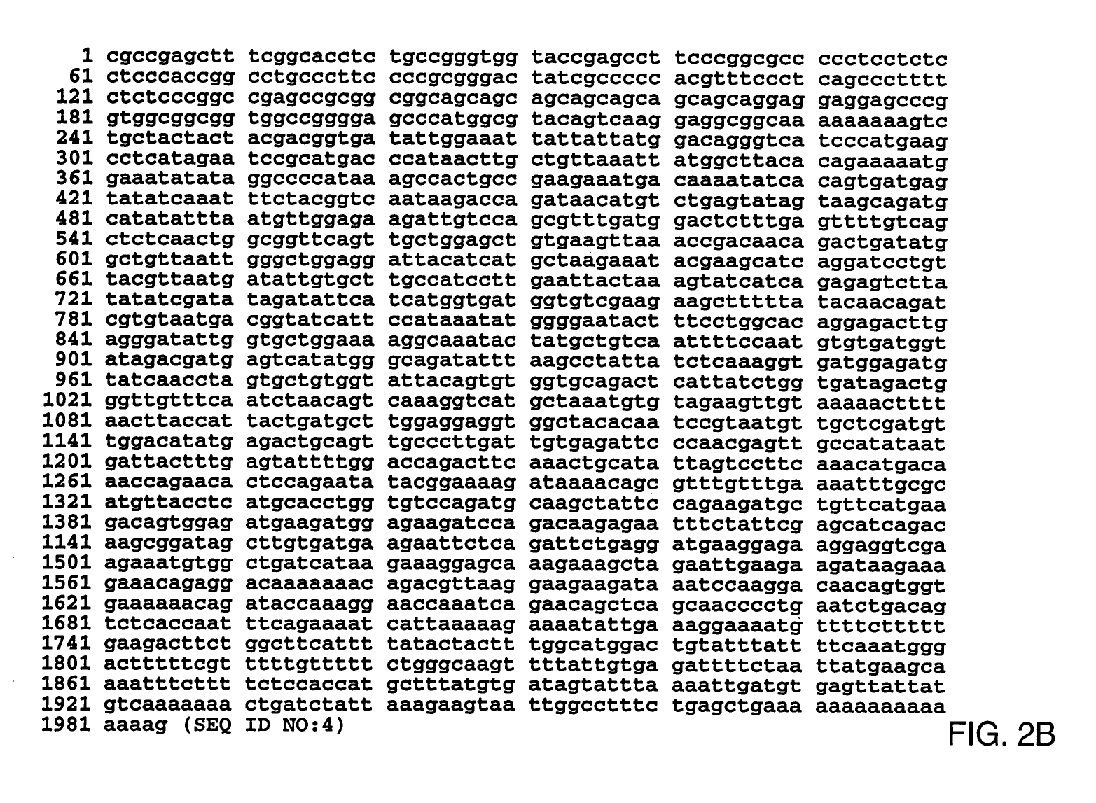

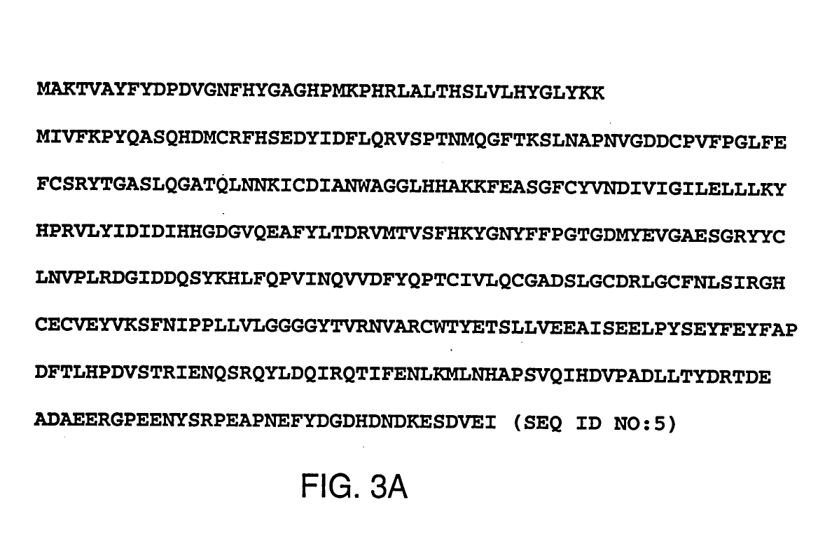

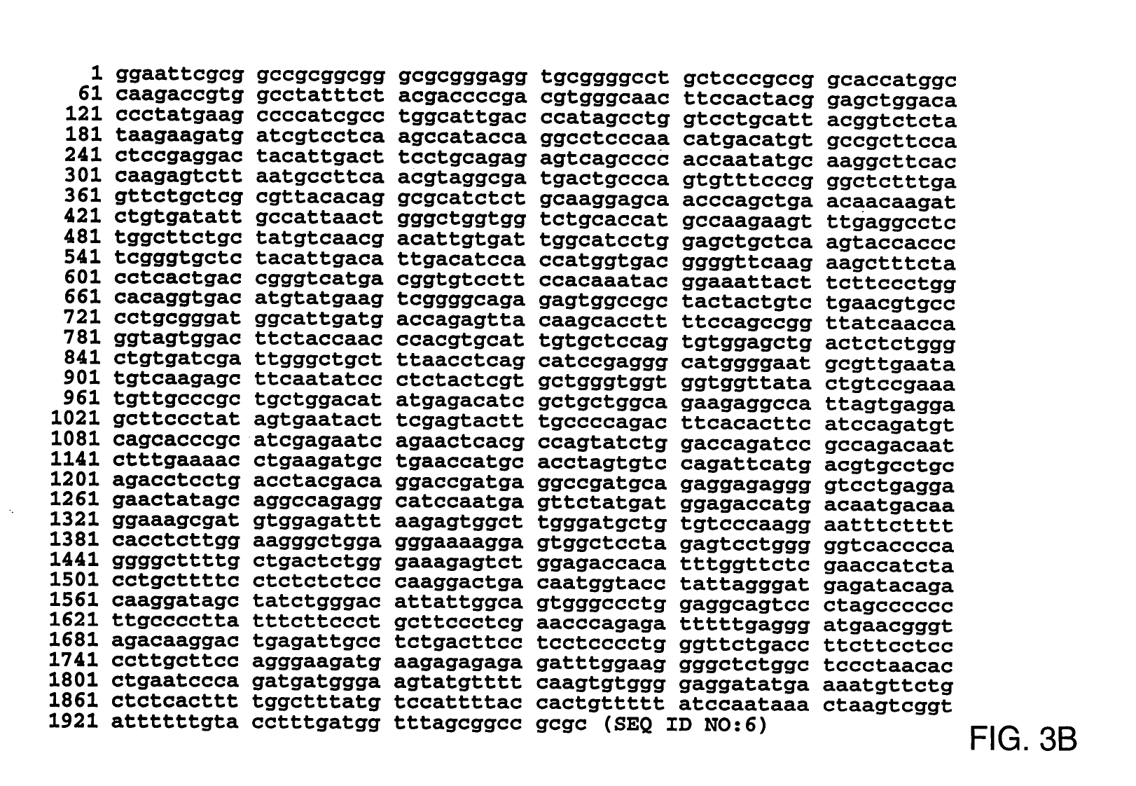

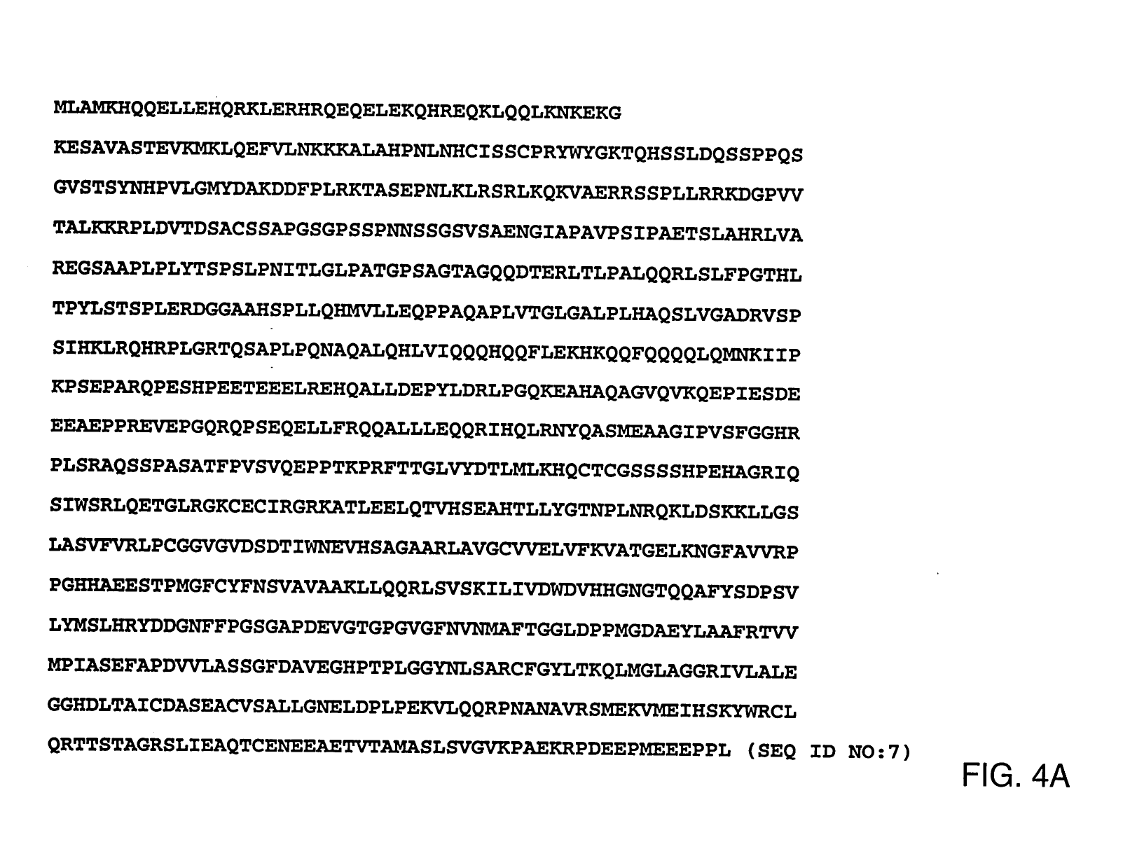

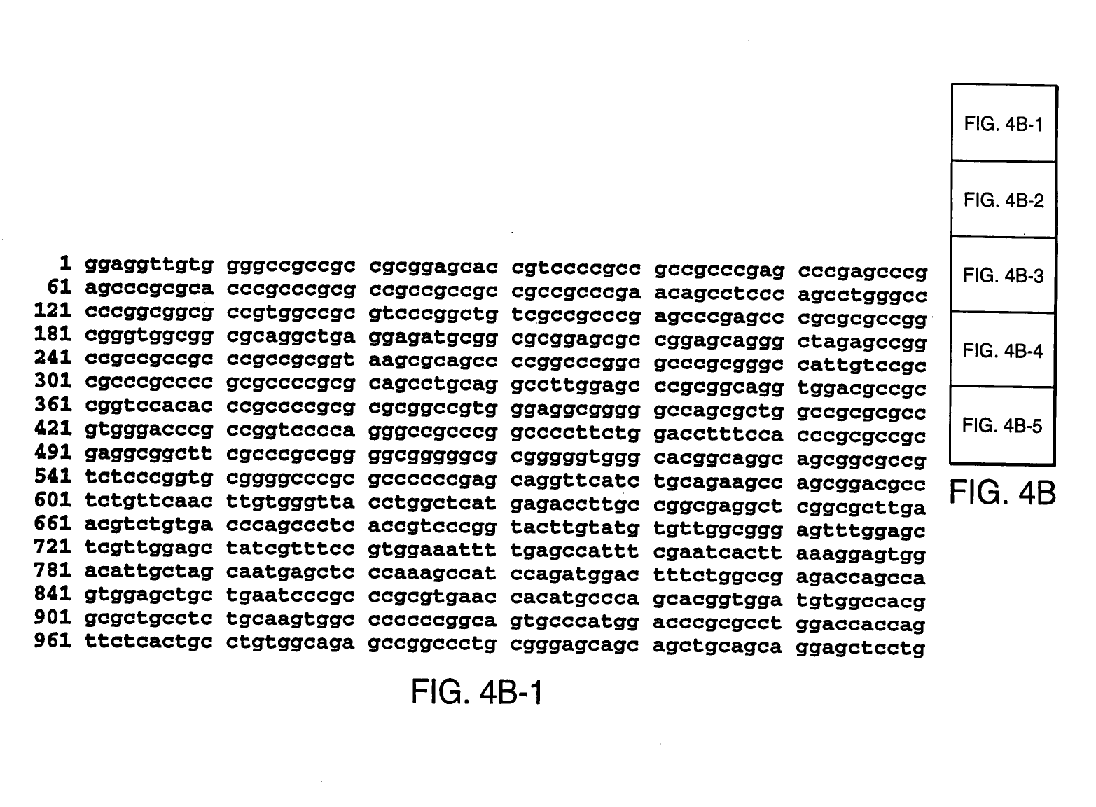

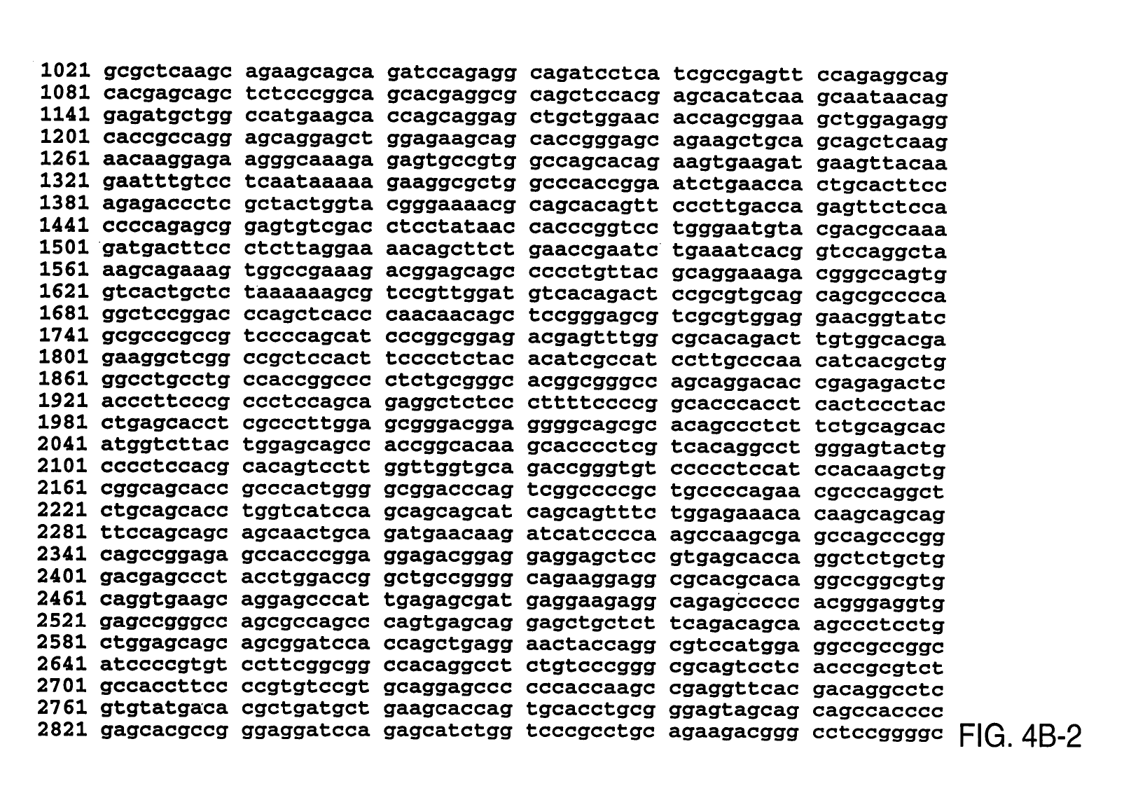

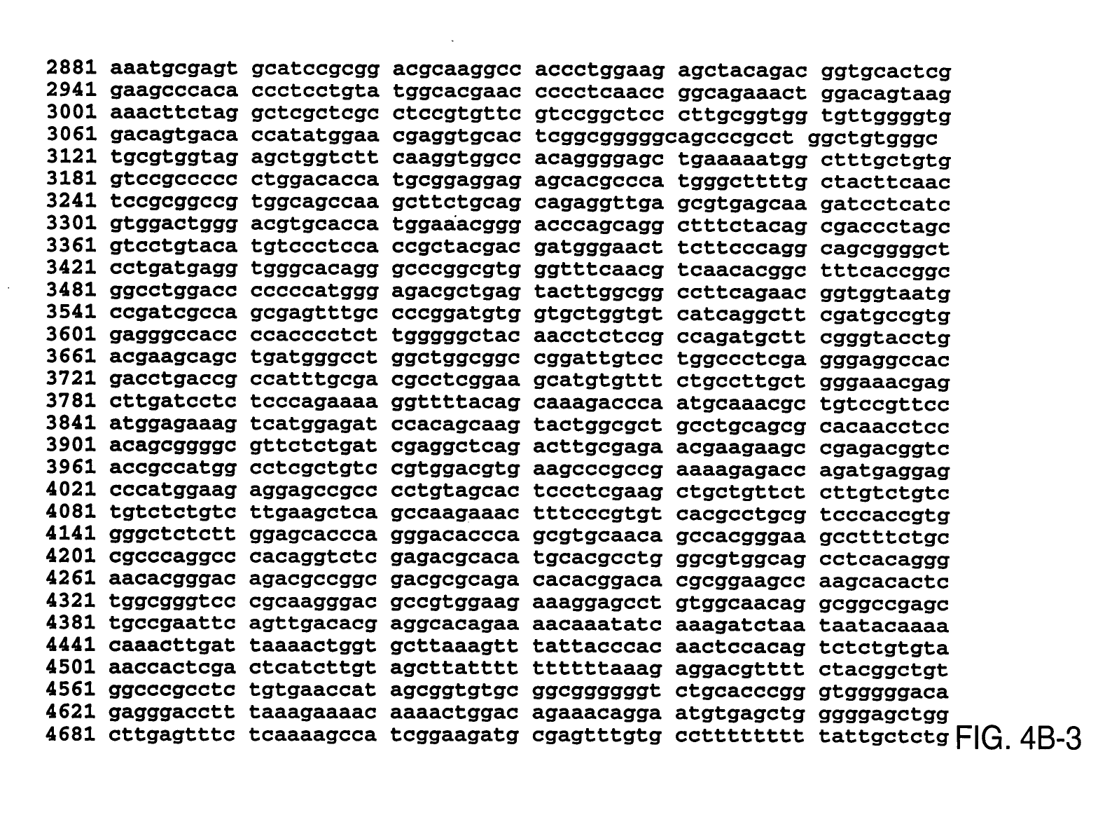

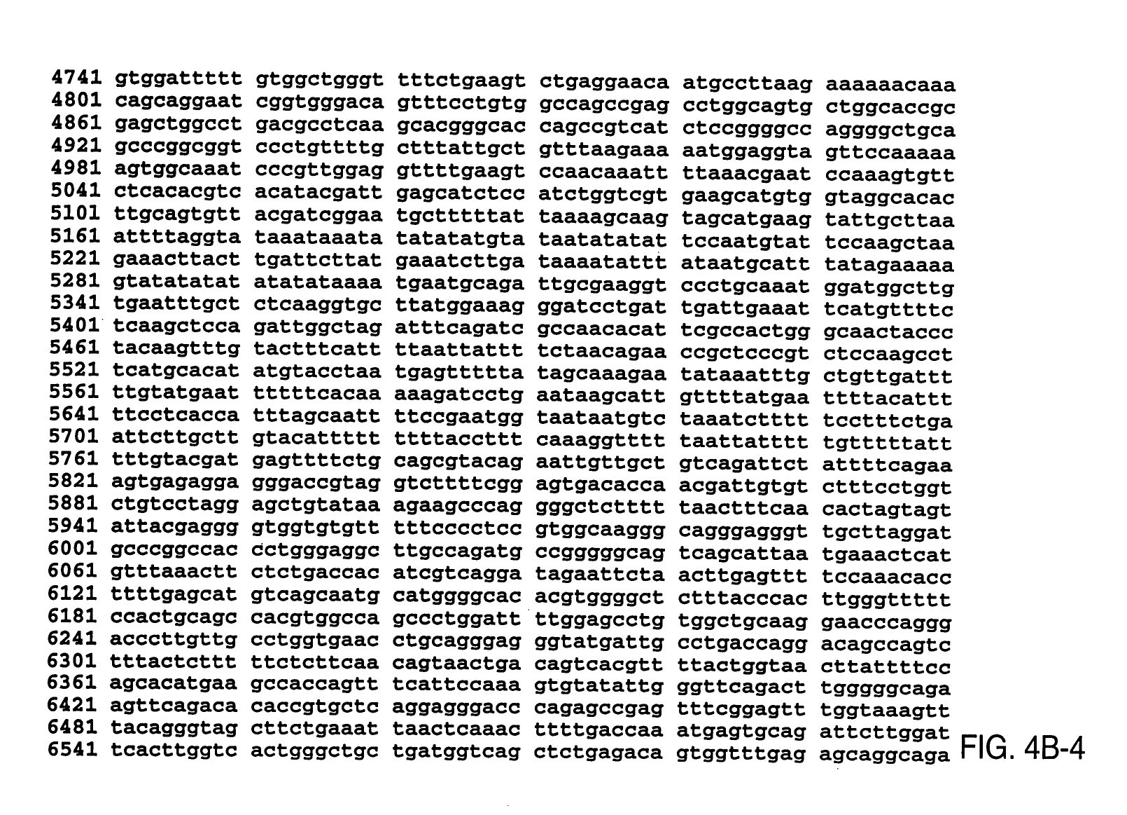

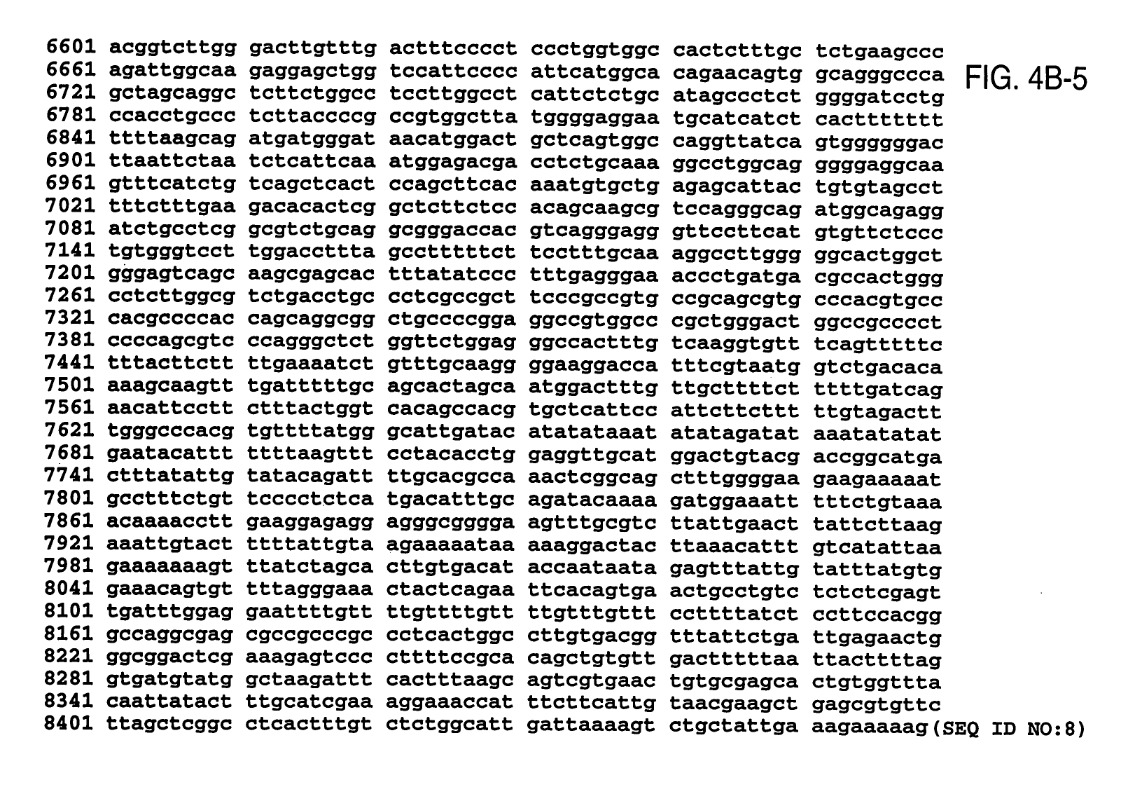

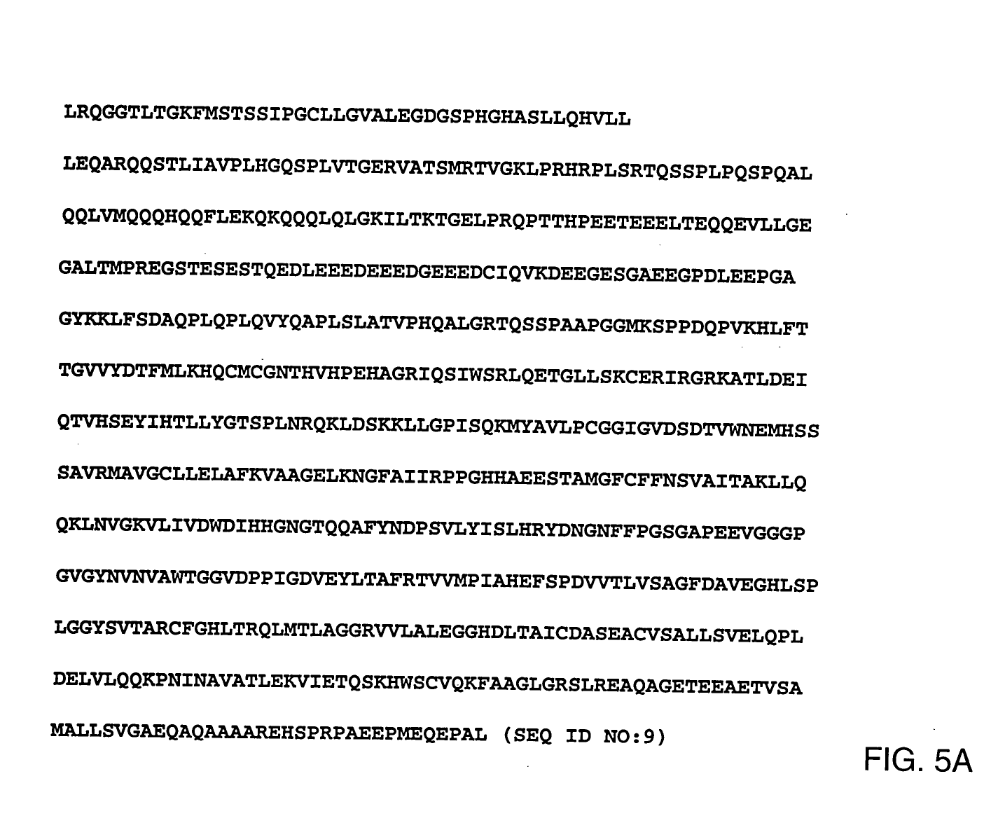

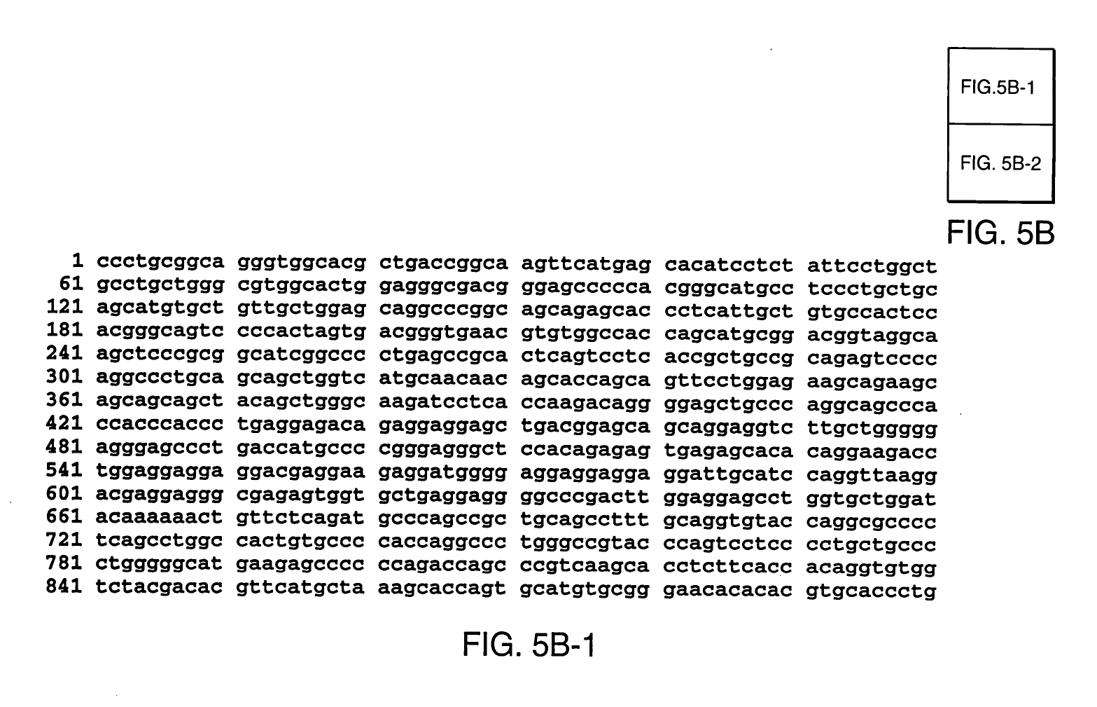

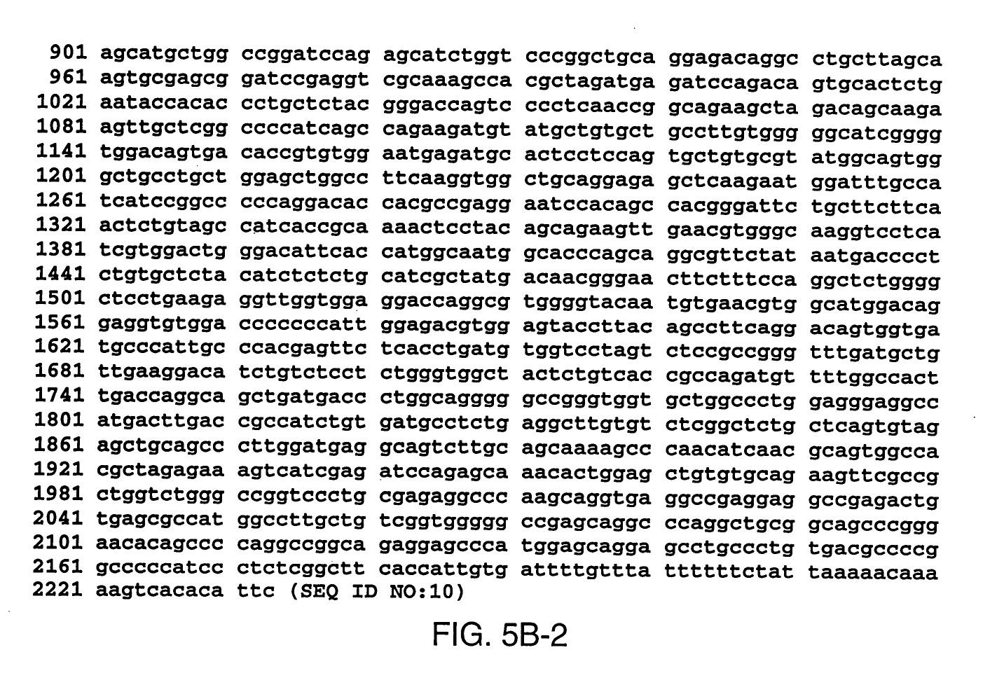

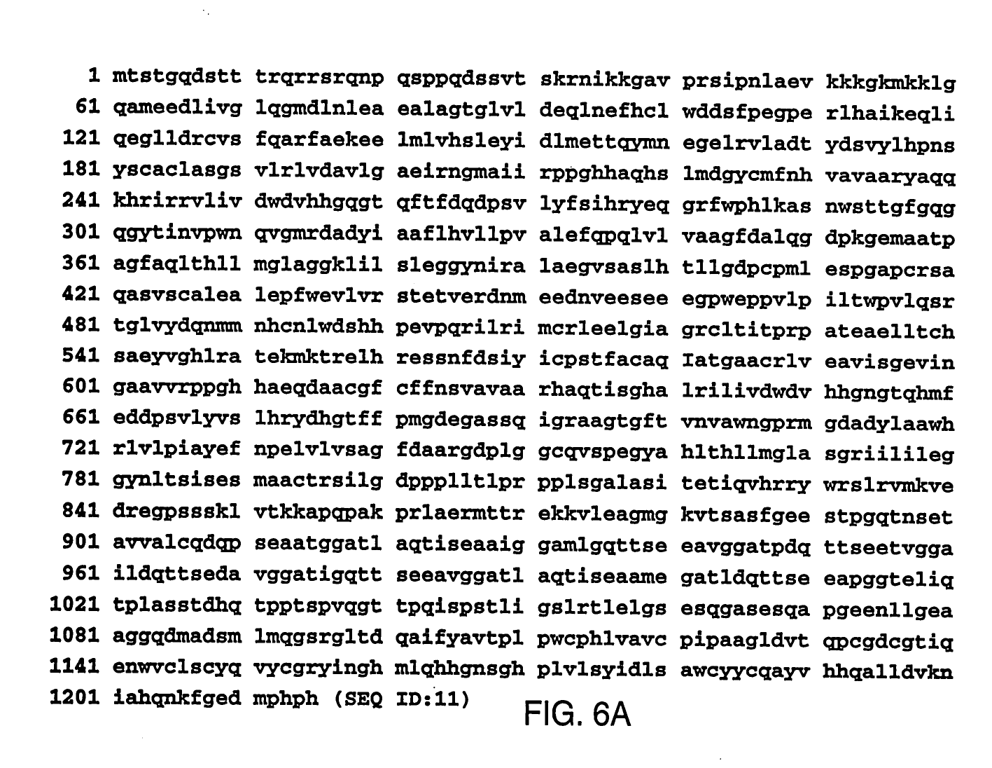

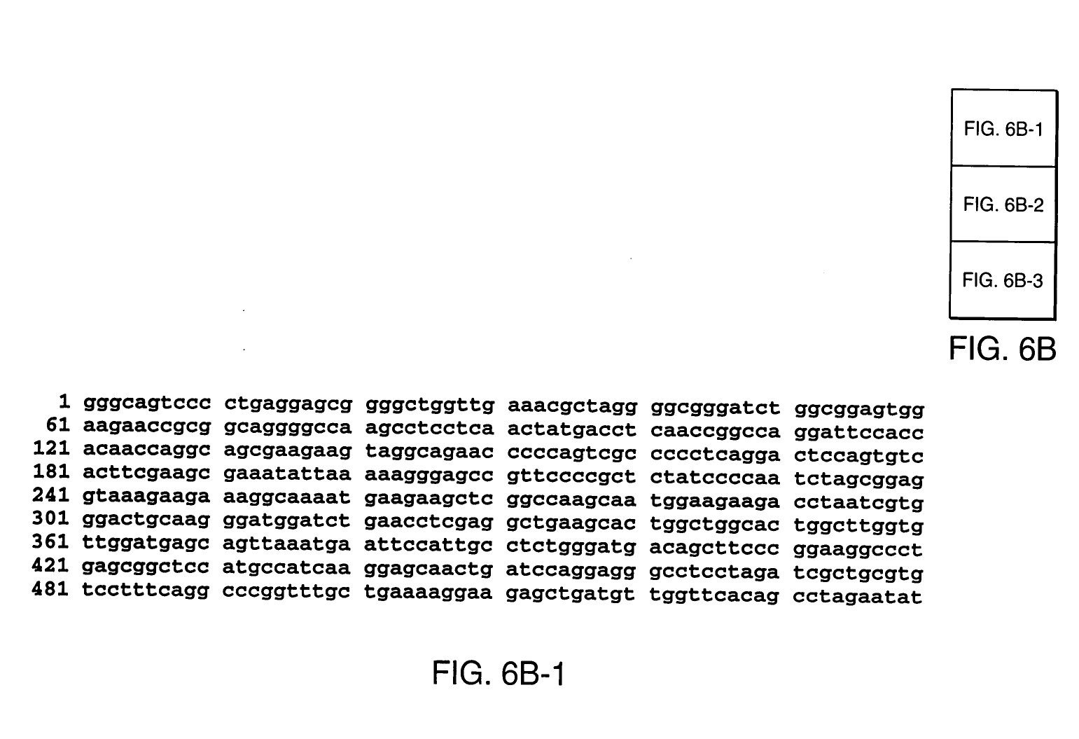

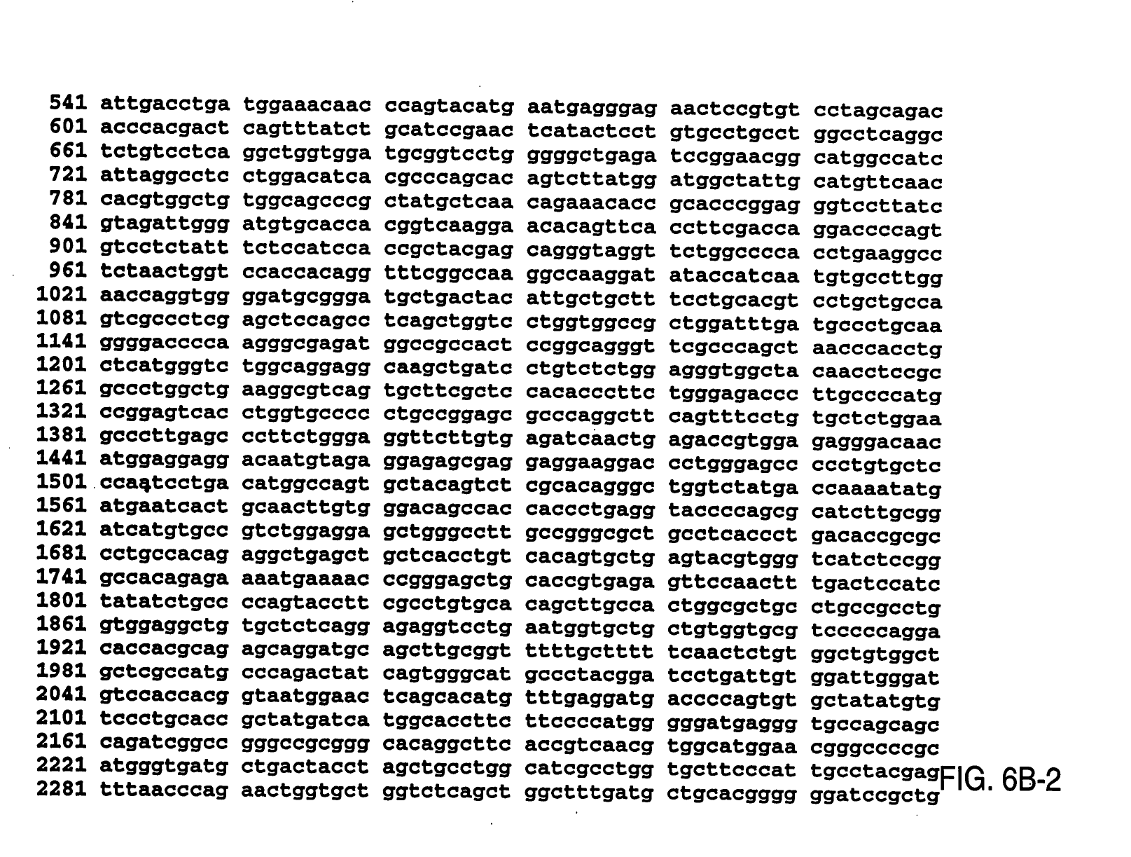

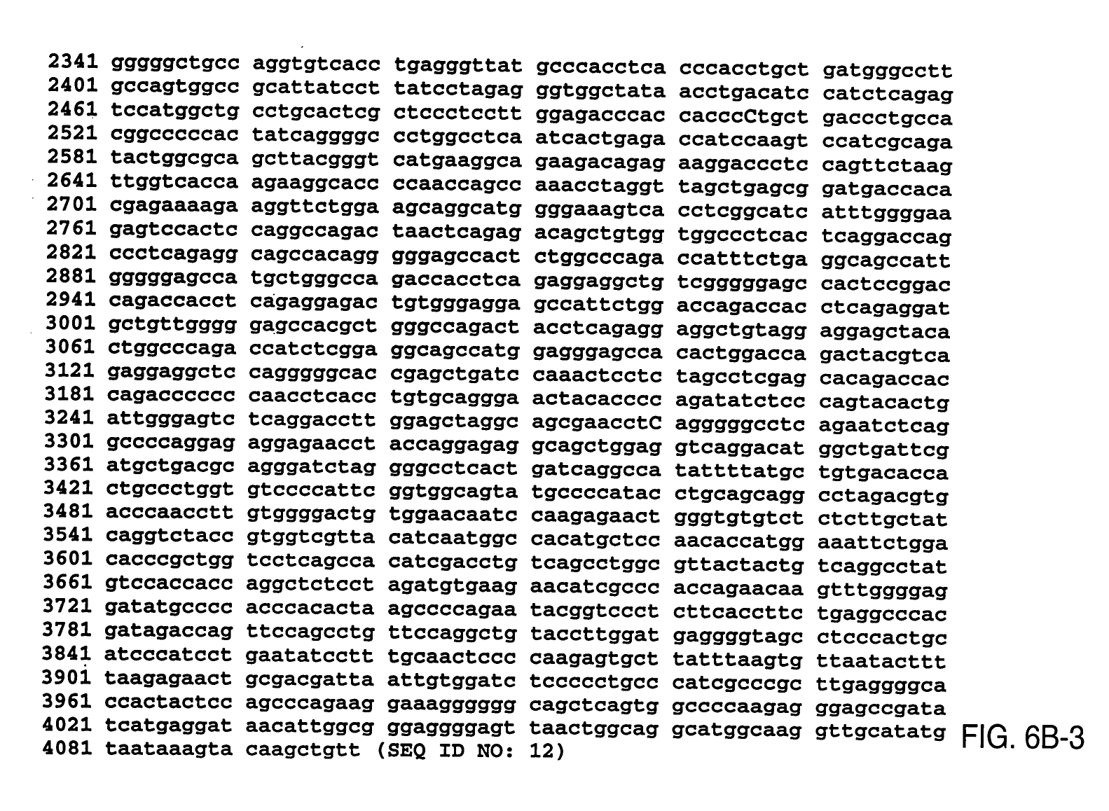

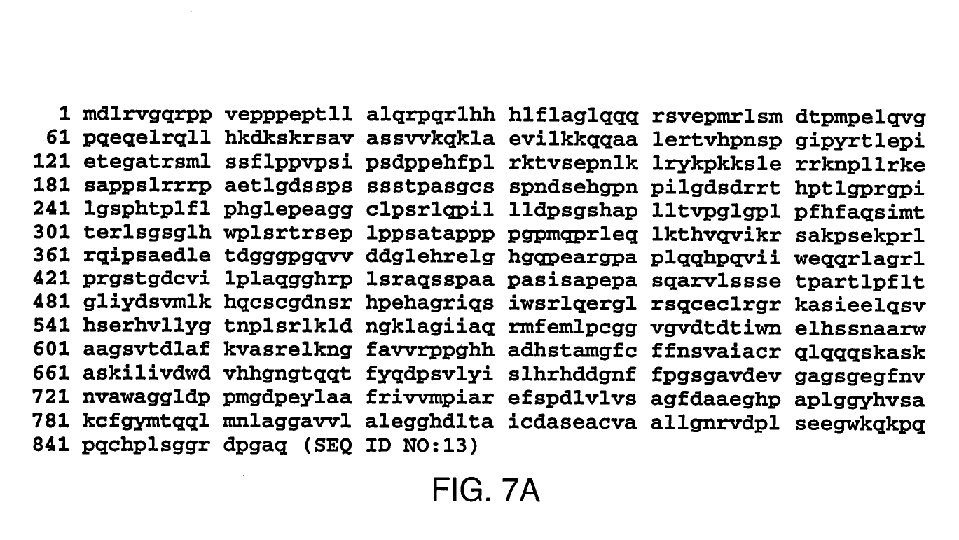

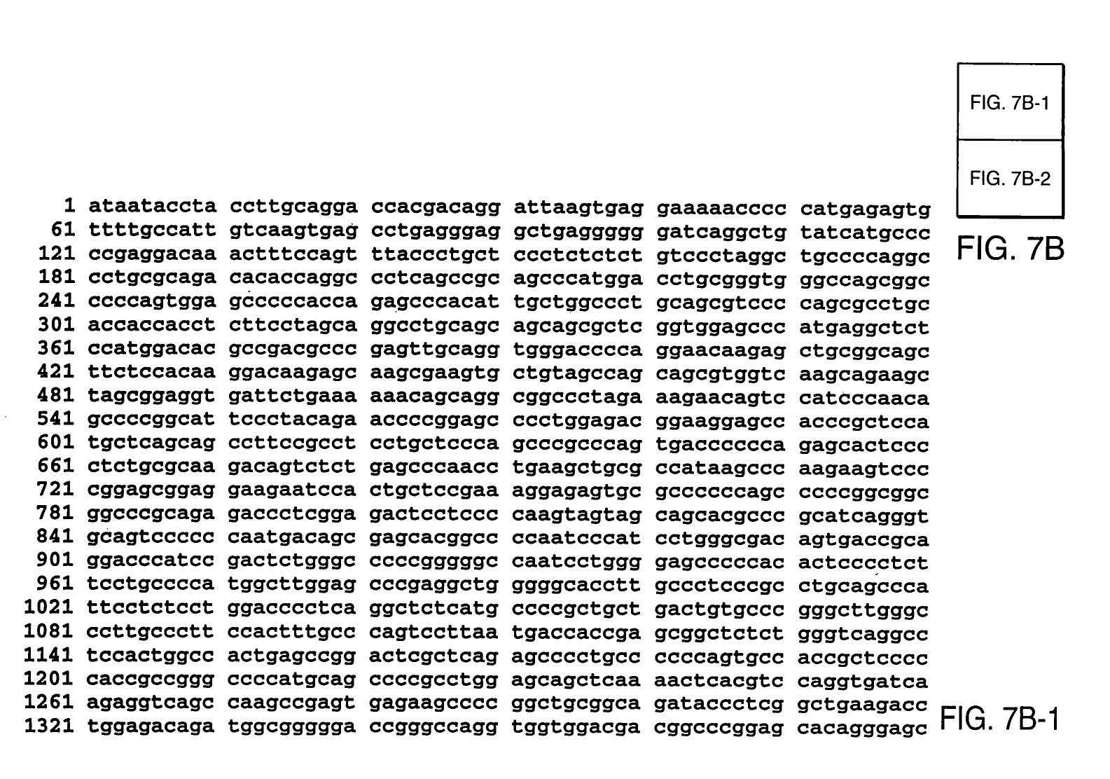

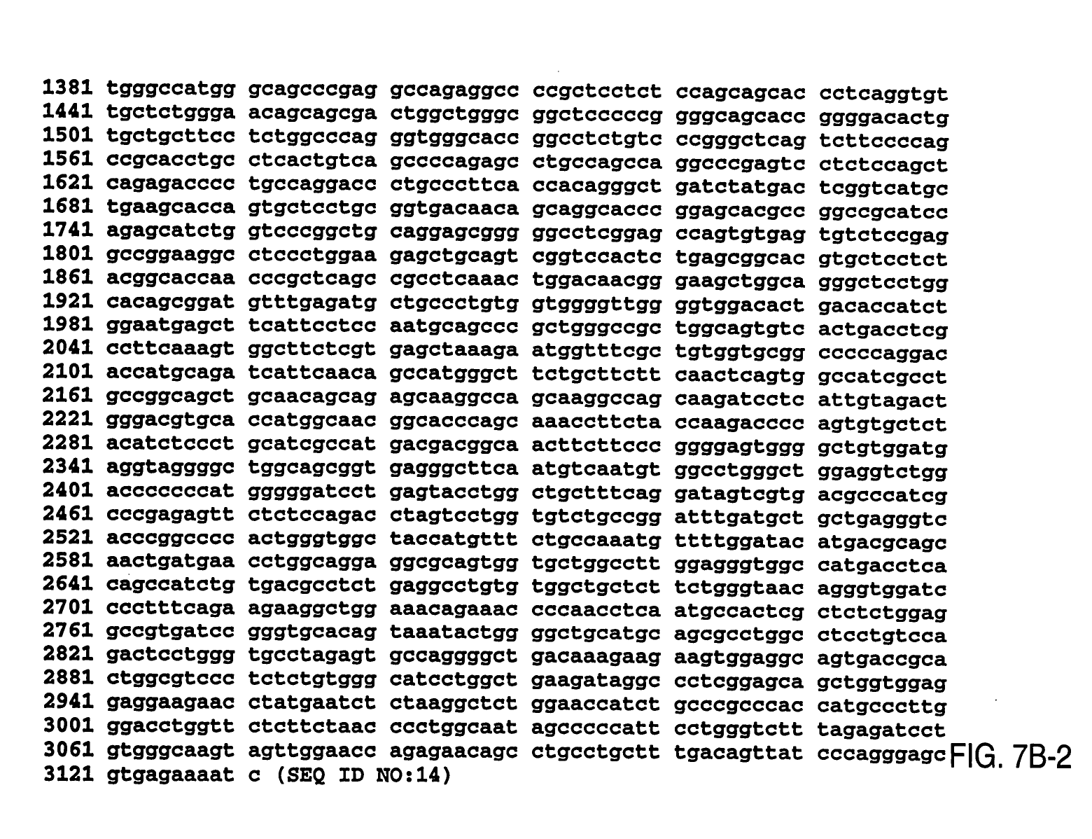

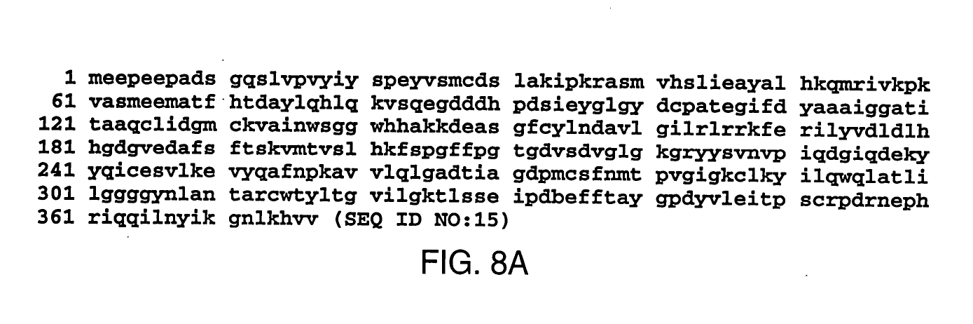

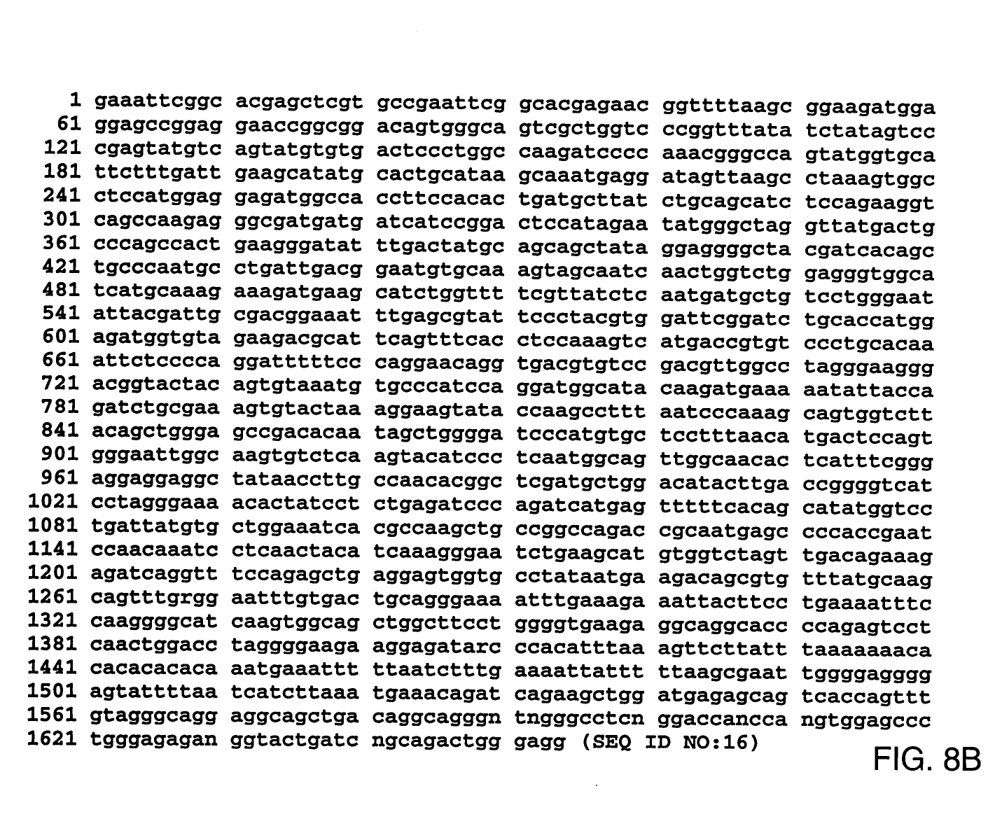

Particularly preferred non-limiting examples of antisense oligonucleotides of the invention are complementary to regions of RNA or double-stranded DNA encoding a histone deacetylase isoform (e.g., HDAC-1, HDAC-2, HDAC-3, HDAC-4, HDAC-5, HDAC-6, HDAC-7, or HDAC-8). (see e.g., GenBank Accession No. U50079 for human HDAC-1 (FIG. 1B); GenBank Accession No. U31814 for human HDAC-2; (FIG. 2B) GenBank Accession No. U75697 for human HDAC-3 (FIG. 3B; GenBank Accession No. AB006626 for human HDAC-4 (FIG. 4B); GenBank Accession No. AB011172 for human HDAC-5 (FIG. 5B); GenBank Accession No. AJ011972 for human HDAC-6 (FIG. 6B); GenBank Accession No. AF239243 for human HDAC-7 (FIG. 7B); and GenBank Accession No. AF230097 for human HDAC-8 (FIG. 8B)).

The sequences encoding histone deacetylases from many non-human animal species are also known (see, for example, GenBank Accession Numbers X98207 (murine HDAC-1); NM_{—}008229 (murine HDAC-2); NM_{—}010411 (murine HDAC-3); NM_{—}006037 (murine HDAC-4); NM_{—}010412 (murine HDAC-5); NM_{—}010413 (murine HDAC-6); and AF207749 (murine HDAC-7)). Accordingly, the antisense oligonucleotides of the invention may also be complementary to regions of RNA or double-stranded DNA that encode histone deacetylases from non-human animals. Antisense oligonucleotides according to these embodiments are useful as tools in animal models for studying the role of specific histone deacetylase isoforms.

Particularly, preferred oligonucleotides have nucleotide sequences of from about 13 to about 35 nucleotides which include the nucleotide sequences shown in Table I. Yet additional particularly preferred oligonucleotides have nucleotide sequences of from about 15 to about 26 nucleotides of the nucleotide sequences shown below. Most preferably, the oligonucleotides shown below have phosphorothioate backbones, are 20-26 nucleotides in length, and are modified such that the terminal four nucleotides at the 5′ end of the oligonucleotide and the terminal four nucleotides at the 3′ end of the oligonucleotide each have 2′-O-methyl groups attached to their sugar residues.

Antisense oligonucleotides used in the present study are shown in Table I.

TABLE 1

Sequences of Human Isotype-Specific Antisense (AS)
Oligonucleotides and Their Mismatch (MM) Oligonucleotides

| | | | | | |
|---|---|---|---|---|---|
| | | Accession | Nucleotide | | Gene |
| Oligo | Target | Number | Position | Sequence | Position |

| | | | | | | |
|---|---|---|---|---|---|---|
| HDAC1 AS1 | Human HDAC1 | U50079 | 1585-1604 | 5′-GAAACGTGAGGGACTCAGCA-3′ | (SEQ ID NO:17) | 3′-UTR |
| HDAC1 AS2 | Human HDAC1 | U50079 | 1565-1584 | 5′-GGAAGCCAGAGCTGGAGAGG-3′ | (SEQ ID NO:18) | 3′-UTR |
| HDAC1 MM | Human HDAC1 | U50079 | 1585-1604 | 5′-GTTAGGTGAGGCACTGAGGA-3′ | (SEQ ID NO:19) | 3′-UTR |
| HDAC2 AS | Human HDAC2 | U31814 | 1643-1622 | 5′-GCTGAGCTGTTCTGATTTGG-3′ | (SEQ ID NO:20) | 3′-UTR |
| HDAC2 MM | Human HDAC2 | U31814 | 1643-1622 | 5′-CGTGAGCACTTCTCATTTCC-3′ | (SEQ ID NO:21) | 3′-UTR |
| HDAC3 AS | Human HDAC3 | AF039703 | 1276-1295 | 5′-CGCTTTCCTTGTCATTGACA-3′ | (SEQ ID NO:22) | 3′-UTR |
| HDAC3 MM | Human HDAC3 | AF039703 | 1276-1295 | 5′-GCCTTTCCTACTCATTGTGT-3′ | (SEQ ID NO:23) | 3′-UTR |
| HDAC4 AS1 | Human HDAC4 | AB006626 | 514-33 | 5-GCTGCCTGCCGTGCCCACCC-3′ | (SEQ ID NO:24) | 5′-UTR |
| HDAC4 MM1 | Human HDAC4 | AB006626 | 514-33 | 5′-CGTGCCTGCGCTGCCCACGG-3′ | (SEQ ID NO:25) | 5′-UTR |
| HDAC4 AS2 | Human HDAC4 | AB006626 | 7710-29 | 5′-TACAGTCCATGCAACCTCCA-3′ | (SEQ ID NO:26) | 3′-UTR |
| HDAC4 MM4 | Human HDAC4 | AB006626 | 7710-29 | 5′-ATCAGTCCAACCAACCTCGT-3′ | (SEQ ID NO:27) | 3′-UTR |
| HDAC5 AS | Human HDAC5 | AF039691 | 2663-2682 | 5′-CTTCGGTCTCACCTGCTTGG-3′ | (SEQ ID NO:28) | 3′-UTR |
| HDAC6 AS | Human HDAC6 | AJ011972 | 3791-3810 | 5′-CAGGCTGGAATGAGCTACAG-3′ | (SEQ ID NO:29) | 3′-UTR |
| HDAC6 MM | Human HDAC6 | AJ011972 | 3791-3810 | 5′-GACGCTGCAATCAGGTAGAC-3′ | (SEQ ID NO:30) | 3′-UTR |
| HDAC7 AS | Human HDAC7 | AF239243 | 2896-2915 | 5′-CTTCAGCCAGGATGCCCACA-3′ | (SEQ ID NO:31) | 3′-UTR |
| HDAC8 AS1 | Human HDAC8 | AF230097 | 51-70 | 5′-CTCCGGCTCCTCCATGTTCC-3′ | (SEQ ID NO:32) | 5′-UTR |
| HDAC8 AS2 | Human HDAC8 | AF230097 | 1328-1347 | 5′-AGCCAGCTGCCACTTGATGC-3′ | (SEQ ID NO:33) | 3′-UTR |

The antisense oligonucleotides according to the invention may optionally be formulated with any of the well known pharmaceutically acceptable carriers or diluents (see preparation of pharmaceutically acceptable formulations in, e.g., Remington's Pharmaceutical Sciences, 18th Edition, ed. A. Gennaro, Mack Publishing Co., Easton, Pa., 1990), with the proviso that such carriers or diluents not affect their ability to modulate HDAC activity.

By way of non-limiting example, the agent of the first aspect of the invention may also be a small molecule inhibitor. The term “small molecule” as used in reference to the inhibition of histone deacetylase is used to identify a compound having a molecular weight preferably less than 1000 Da, more preferably less than 800 Da, and most preferably less than 600 Da, which is capable of interacting with a histone deacetylase and inhibiting the expression of a nucleic acid molecule encoding an HDAC isoform or activity of an HDAC protein. Inhibiting histone deacetylase enzymatic activity means reducing the ability of a histone deacetylase to remove an acetyl group from a histone. In some preferred embodiments, such reduction of histone deacetylase activity is at least about 50%, more preferably at least about 75%, and still more preferably at least about 90%. In other preferred embodiments, histone deacetylase activity is reduced by at least 95% and more preferably by at least 99%. In one certain embodiment, the small molecule inhibitor is an inhibitor of one or more but less than all HDAC isoforms. By “all HDAC isoforms” is meant all proteins that specifically remove an epsilon acetyl group from an N-terminal lysine of a histone, and includes, without limitation, HDAC-1, HDAC-2, HDAC-3, HDAC-4, HDAC-5, HDAC-6, HDAC-7, or HDAC-8, all of which are considered “related proteins,” as used herein.

Most preferably, a histone deacetylase small molecule inhibitor interacts with and reduces the activity of one or more histone deacetylase isoforms (e.g., HDAC-1 and/or HDAC-4), but does not interact with or reduce the activities of all of the other histone deacetylase isoforms (e.g., HDAC-2 and HDAC-6). As discussed below, a preferred histone deacetylase small molecule inhibitor is one that interacts with and reduces the enzymatic activity of a histone deacetylase isoform that is involved in tumorigenesis.

Non-limiting examples of small molecule inhibitors useful for the invention are presented in Table 2.

TABLE 2
Small Molecule HDAC Inhibitors [μM] and Their Antitumor Activities In Vivo

| | | | | | | | | % inhibitor of tumor formation in vivo | | | | |
|---|---|---|---|---|---|---|---|---|---|---|---|---|
| | | | Enzyme | | | | | | Cell | | | |
| | | | IC50 (μM) | | | | | | Cycle | | | |
| | Inhibitor | HD- | | HD- | HD- | HD- | H4- | | Arrest | | | |
| Cpd | Structure | AC1 | | AC3 | AC4 | AC6 | Ac | MTT | EC | colon | lung | prostate |
| 1 | 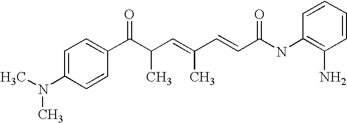 | 3 | 25 | 21 | 23 | >50 | 1 | 3 | 2 | | | |
| 2 | 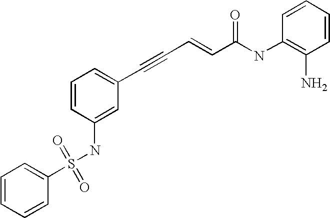 | 3 | 31 | 30 | 35 | >30 | 5 | 4 | 8 | 53 | 54 | |
| | | | | | | | | | | (40,po) | (50,ip) | |
| 3 | 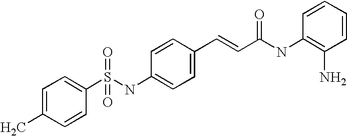 | 3 | 22 | 45 | 28 | >50 | 5 | 4 | 2 | 55 | | |
| | | | | | | | | | | (40,ip) | | |

note: for in vivo antitumor studies, numbers outside brackets indicate % of inhibition of tumor growth in vivo; numbers in brackets indicate daily dose of inhibitor used (mg/kg body weight/day); oral (PO) or intraperitoneal (IP) administration is indicated in brackets.

The reagents according to the invention are useful as analytical tools and as therapeutic tools, including as gene therapy tools. The invention also provides methods and compositions which may be manipulated and fine-tuned to fit the condition(s) to be treated while producing fewer side effects.

In a second aspect, the invention provides a method for inhibiting one or more, but less than all, histone deacetylase isoforms in a cell comprising contacting the cell with an agent of the first aspect of the invention. By way of non-limiting example, the agent may be an antisense oligonucleotide or a small molecule inhibitor that inhibits the expression of one or more, but less than all, specific histone deacetylase isoforms in the cell.

In one certain embodiment, the invention provides a method comprising contacting a cell with an antisense oligonucleotide that inhibits one or more but less than all histone deacetylase isoforms in the cell. Preferably, cell proliferation is inhibited in the contacted cell. Thus, the antisense oligonucleotides according to the invention are useful in therapeutic approaches to human diseases including benign and malignant neoplasms by inhibiting cell proliferation in cells contacted with the antisense oligonucleotides. The phrase “inhibiting cell proliferation” is used to denote an ability of a histone deacetylase antisense oligonucleotide or a small molecule histone deacetylase inhibitor (or combination thereof) to retard the growth of cells contacted with the oligonucleotide or small molecule inhibitor, as compared to cells not contacted. Such an assessment of cell proliferation can be made by counting contacted and non-contacted cells using a Coulter Cell Counter (Coulter, Miami, Fla.) or a hemacytometer. Where the cells are in a solid growth (e.g., a solid tumor or organ), such an assessment of cell proliferation can be made by measuring the growth with calipers, and comparing the size of the growth of contacted cells with non-contacted cells. Preferably, the term includes a retardation of cell proliferation that is at least 50% of non-contacted cells. More preferably, the term includes a retardation of cell proliferation that is 100% of non-contacted cells (i.e., the contacted cells do not increase in number or size). Most preferably, the term includes a reduction in the number or size of contacted cells, as compared to non-contacted cells. Thus, a histone deacetylase antisense oligonucleotide or a histone deacetylase small molecule inhibitor that inhibits cell proliferation in a contacted cell may induce the contacted cell to undergo growth retardation, to undergo growth arrest, to undergo programmed cell death (i.e., to apoptose), or to undergo necrotic cell death.

Conversely, the phrase “inducing cell proliferation” and similar terms are used to denote the requirement of the presence or enzymatic activity of a specific histone deacetylase isoform for cell proliferation in a normal (i.e., non-neoplastic) cell. Hence, over-expression of a specific histone deacetylase isoform that induces cell proliferation may or may not lead to increased cell proliferation; however, inhibition of a specific histone deacetylase isoform that induces cell proliferation will lead to inhibition of cell proliferation.

The cell proliferation inhibiting ability of the antisense oligonucleotides according to the invention allows the synchronization of a population of a-synchronously growing cells. For example, the antisense oligonucleotides of the invention may be used to arrest a population of non-neoplastic cells grown in vitro in the G1 or G2 phase of the cell cycle. Such synchronization allows, for example, the identification of gene and/or gene products expressed during the G1 or G2 phase of the cell cycle. Such a synchronization of cultured cells may also be useful for testing the efficacy of a new transfection protocol, where transfection efficiency varies and is dependent upon the particular cell cycle phase of the cell to be transfected. Use of the antisense oligonucleotides of the invention allows the synchronization of a population of cells, thereby aiding detection of enhanced transfection efficiency.

The anti-neoplastic utility of the antisense oligonucleotides according to the invention is described in detail elsewhere in this specification.

In yet other preferred embodiments, the cell contacted with a histone deacetylase antisense oligonucleotide is also contacted with a histone deacetylase small molecule inhibitor.

In a few preferred embodiments, the histone deacetylase small molecule inhibitor is operably associated with the antisense oligonucleotide. As mentioned above, the antisense oligonucleotides according to the invention may optionally be formulated well known pharmaceutically acceptable carriers or diluents. This formulation may further contain one or more one or more additional histone deacetylase antisense oligonucleotide(s), and/or one or more histone deacetylase small molecule inhibitor(s), or it may contain any other pharmacologically active agent.

In a particularly preferred embodiment of the invention, the antisense oligonucleotide is in operable association with a histone deacetylase small molecule inhibitor. The term “operable association” includes any association between the antisense oligonucleotide and the histone deacetylase small molecule inhibitor which allows an antisense oligonucleotide to inhibit one or more specific histone deacetylase isoform-encoding nucleic acid expression and allows the histone deacetylase small molecule inhibitor to inhibit specific histone deacetylase isoform enzymatic activity. One or more antisense oligonucleotide of the invention may be operably associated with one or more histone deacetylase small molecule inhibitor. In some preferred embodiments, an antisense oligonucleotide of the invention that targets one particular histone deacetylase isoform (e.g., HDAC-1) is operably associated with a histone deacetylase small molecule inhibitor which targets the same histone deacetylase isoform. A preferred operable association is a hydrolyzable. Preferably, the hydrolyzable association is a covalent linkage between the antisense oligonucleotide and the histone deacetylase small molecule inhibitor. Preferably, such covalent linkage is hydrolyzable by esterases and/or amidases. Examples of such hydrolyzable associations are well known in the art. Phosphate esters are particularly preferred.

In certain preferred embodiments, the covalent linkage may be directly between the antisense oligonucleotide and the histone deacetylase small molecule inhibitor so as to integrate the histone deacetylase small molecule inhibitor into the backbone. Alternatively, the covalent linkage may be through an extended structure and may be formed by covalently linking the antisense oligonucleotide to the histone deacetylase small molecule inhibitor through coupling of both the antisense oligonucleotide and the histone deacetylase small molecule inhibitor to a carrier molecule such as a carbohydrate, a peptide or a lipid or a glycolipid. Other preferred operable associations include lipophilic association, such as formation of a liposome containing an antisense oligonucleotide and the histone deacetylase small molecule inhibitor covalently linked to a lipophilic molecule and thus associated with the liposome. Such lipophilic molecules include without limitation phosphotidylcholine, cholesterol, phosphatidylethanolamine, and synthetic neoglycolipids, such as syalyllacNAc-HDPE. In certain preferred embodiments, the operable association may not be a physical association, but simply a simultaneous existence in the body, for example, when the antisense oligonucleotide is associated with one liposome and the small molecule inhibitor is associated with another liposome.

In a third aspect, the invention provides a method for inhibiting neoplastic cell proliferation in an animal comprising administering to an animal having at least one neoplastic cell present in its body a therapeutically effective amount of an agent of the first aspect of the invention. In one certain embodiment, the agent is an antisense oligonucleotide of the first aspect of the invention, and the method further comprises a pharmaceutically acceptable carrier. The antisense oligonucleotide and the pharmaceutically acceptable carrier are administered for a therapeutically effective period of time. Preferably, the animal is a mammal, particularly a domesticated mammal. Most preferably, the animal is a human.

The term “neoplastic cell” is used to denote a cell that shows aberrant cell growth. Preferably, the aberrant cell growth of a neoplastic cell is increased cell growth. A neoplastic cell may be a hyperplastic cell, a cell that shows a lack of contact inhibition of growth in vitro, a benign tumor cell that is incapable of metastasis in vivo, or a cancer cell that is capable of metastases in vivo and that may recur after attempted removal. The term “tumorigenesis” is used to denote the induction of cell proliferation that leads to the development of a neoplastic growth.

The terms “therapeutically effective amount” and “therapeutically effective period of time” are used to denote known treatments at dosages and for periods of time effective to reduce neoplastic cell growth. Preferably, such administration should be parenteral, oral, sublingual, transdermal, topical, intranasal, or intrarectal. When administered systemically the therapeutic composition is preferably administered at a sufficient dosage to attain a blood level of antisense oligonucleotide from about 0.1 μM to about 10 μM. For localized administration, much lower concentrations than this may be effective, and much higher concentrations may be tolerated. One of skill in the art will appreciate that such therapeutic effect resulting in a lower effective concentration of the histone deacetylase inhibitor may vary considerably depending on the tissue, organ, or the particular animal or patient to be treated according to the invention.

In a preferred embodiment, the therapeutic composition of the invention is administered systemically at a sufficient dosage to attain a blood level of antisense oligonucleotide from about 0.01 μM to about 20 μM. In a particularly preferred embodiment, the therapeutic composition is administered at a sufficient dosage to attain a blood level of antisense oligonucleotide from about 0.05 μM to about 15 μM. In a more preferred embodiment, the blood level of antisense oligonucleotide is from about 0.1 μM to about 10 μM.

For localized administration, much lower concentrations than this may be therapeutically effective. Preferably, a total dosage of antisense oligonucleotide will range from about 0.1 mg to about 200 mg oligonucleotide per kg body weight per day. In a more preferred embodiment, a total dosage of antisense oligonucleotide will range from about 1 mg to about 20 mg oligonucleotide per kg body weight per day. In a most preferred embodiment, a total dosage of antisense oligonucleotide will range from about 1 mg to about 10 mg oligonucleotide per kg body weight per day. In a particularly preferred embodiment, the therapeutically effective amount of a histone deacetylase antisense oligonucleotide is about 5 mg oligonucleotide per kg body weight per day.

In certain preferred embodiments of the third aspect of the invention, the method further comprises administering to the animal a therapeutically effective amount of a histone deacetylase small molecule inhibitor with a pharmaceutically acceptable carrier for a therapeutically effective period of time. In some preferred embodiments, the histone deacetylase small molecule inhibitor is operably associated with the antisense oligonucleotide, as described supra.

The histone deacetylase small molecule inhibitor-containing therapeutic composition of the invention is administered systemically at a sufficient dosage to attain a blood level histone deacetylase small molecule inhibitor from about 0.01 μM to about 10 μM. In a particularly preferred embodiment, the therapeutic composition is administered at a sufficient dosage to attain a blood level of histone deacetylase small molecule inhibitor from about 0.05 μM to about 10 μM. In a more preferred embodiment, the blood level of histone deacetylase small molecule inhibitor is from about 0.1 μM to about 5 μM. For localized administration, much lower concentrations than this may be effective. Preferably, a total dosage of histone deacetylase small molecule inhibitor will range from about 0.01 mg to about 100 mg protein effector per kg body weight per day. In a more preferred embodiment, a total dosage of histone deacetylase small molecule inhibitor will range from about 0.1 mg to about 50 mg protein effector per kg body weight per day. In a most preferred embodiment, a total dosage of histone deacetylase small molecule inhibitor will range from about 0.1 mg to about 10 mg protein effector per kg body weight per day. In a particularly preferred embodiment, the therapeutically effective synergistic amount of histone deacetylase small molecule inhibitor (when administered with an antisense oligonucleotide) is about 5 mg per kg body weight per day.

Certain preferred embodiments of this aspect of the invention result in an improved inhibitory effect, thereby reducing the therapeutically effective concentrations of either or both of the nucleic acid level inhibitor (i.e., antisense oligonucleotide) and the protein level inhibitor (i.e.,histone deacetylase small molecule inhibitor) required to obtain a given inhibitory effect as compared to those necessary when either is used individually.

Furthermore, one of skill will appreciate that the therapeutically effective synergistic amount of either the antisense oligonucleotide or the histone deacetylase inhibitor may be lowered or increased by fine tuning and altering the amount of the other component. The invention therefore provides a method to tailor the administration/treatment to the particular exigencies specific to a given animal species or particular patient. Therapeutically effective ranges may be easily determined for example empirically by starting at relatively low amounts and by step-wise increments with concurrent evaluation of inhibition.

In a fourth aspect, the invention provides a method for identifying a specific histone deacetylase isoform that is required for induction of cell proliferation comprising contacting a cell with an agent of the first aspect of the invention. In certain preferred embodiments, the agent is an antisense oligonucleotide that inhibits the expression of a histone deacetylase isoform, wherein the antisense oligonucleotide is specific for a particular HDAC isoform, and thus inhibition of cell proliferation in the contacted cell identifies the histone deacetylase isoform as a histone deacetylase isoform that is required for induction of cell proliferation. In other certain embodiments, the agent is a small molecule inhibitor that inhibits the activity of a histone deacetylase isoform, wherein the small molecule inhibitor is specific for a particular HDAC isoform, and thus inhibition of cell proliferation in the contacted cell identifies the histone deacetylase isoform as a histone deacetylase isoform that is required for induction of cell proliferation. In certain preferred embodiments, the cell is a neoplastic cell, and the induction of cell proliferation is tumorigenesis. In still yet other preferred embodiments of the fourth aspect of the invention, the method comprises an agent of the first aspect of the invention which is a combination of one or more antisense oligonucleotides and/or one or more small molecule inhibitors of the first aspect of the invention. In certain preferred embodiments, the histone deacetylase isoform is HDAC-1, HDAC-2, HDAC-3, HDAC-4, HDAC-5, HDAC-6, HDAC-7, or HDAC-8. In other certain preferred embodiments, the histone deacetylase isoform is HDAC-1 and/or HDAC-4.

In an fifth aspect, the invention provides a method for identifying a histone deacetylase isoform that is involved in induction of cell differentiation comprising contacting a cell with an agent that inhibits the expression of a histone deacetylase isoform, wherein induction of differentiation in the contacted cell identifies the histone deacetylase isoform as a histone deacetylase isoform that is involved in induction of cell differentiation. In certain preferred embodiments, the agent is an antisense oligonucleotide of the first aspect of the invention. In other certain preferred embodiments, the agent is an small molecule inhibitor of the first aspect of the invention. In still other certain embodiments, the cell is a neoplastic cell. In still yet other preferred embodiments of the fifth aspect of the invention, the method comprises an agent of the first aspect of the invention which is a combination of one or more antisense oligonucleotides and/or one or more small molecule inhibitors of the first aspect of the invention. In certain preferred embodiments, the histone deacetylase isoform is HDAC-1, HDAC-2, HDAC-3, HDAC-4, HDAC-5, HDAC-6, HDAC-7, or HDAC-8. In other certain preferred embodiments, the histone deacetylase isoform is HDAC-1 and/or HDAC-4.

In a sixth aspect, the invention provides a method for inhibiting neoplastic cell growth in an animal comprising administering to an animal having at least one neoplastic cell present in its body a therapeutically effective amount of an agent of the first aspect of the invention. In certain embodiments thereof, the agent is an antisense oligonucleotide, which is combined with a pharmaceutically acceptable carrier and administered for a therapeutically effective period of time.

In certain embodiments where the agent of the first aspect of the invention is a histone deacetylase small molecule inhibitor, therapeutic compositions of the invention comprising said small molecule inhibitor(s) are administered systemically at a sufficient dosage to attain a blood level histone deacetylase small molecule inhibitor from about 0.01 μM to about 10 μM. In a particularly preferred embodiment, the therapeutic composition is administered at a sufficient dosage to attain a blood level of histone deacetylase small molecule inhibitor from about 0.05 μM to about 10 μM. In a more preferred embodiment, the blood level of histone deacetylase small molecule inhibitor is from about 0.1 μM to about 5 μM. For localized administration, much lower concentrations than this may be effective. Preferably, a total dosage of histone deacetylase small molecule inhibitor will range from about 0.01 mg to about 100 mg protein effector per kg body weight per day. In a more preferred embodiment, a total dosage of histone deacetylase small molecule inhibitor will range from about 0.1 mg to about 50 mg protein effector per kg body weight per day. In a most preferred embodiment, a total dosage of histone deacetylase small molecule inhibitor will range from about 0.1 mg to about 10 mg protein effector per kg body weight per day.

In a sixth aspect, the invention provides a method for investigating the role of a particular histone deacetylase isoform in cellular proliferation, including the proliferation of neoplastic cells. In this method, the cell type of interest is contacted with an amount of an antisense oligonucleotide that inhibits the expression of one or more specific histone deacetylase isoform, as described for the first aspect according to the invention, resulting in inhibition of expression of the histone deacetylase isoform(s) in the cell. If the contacted cell with inhibited expression of the histone deacetylase isoform(s) also shows an inhibition in cell proliferation, then the histone deacetylase isoform(s) is required for the induction of cell proliferation. In this scenario, if the contacted cell is a neoplastic cell, and the contacted neoplastic cell shows an inhibition of cell proliferation, then the histone deacetylase isoform whose expression was inhibited is a histone deacetylase isoform that is required for tumorigenesis. In certain preferred embodiments, the histone deacetylase isoform is HDAC-1, HDAC-2, HDAC-3, HDAC-4, HDAC-5, HDAC-6, HDAC-7, or HDAC-8. In certain preferred embodiments, the histone deacetylase isoform is HDAC-1 and/or HDAC-4.

Thus, by identifying a particular histone deacetylase isoform that is required for in the induction of cell proliferation, only that particular histone deacetylase isoform need be targeted with an antisense oligonucleotide to inhibit cell proliferation or induce differentiation. Consequently, a lower therapeutically effective dose of antisense oligonucleotide may be able to effectively inhibit cell proliferation. Moreover, undesirable side effects of inhibiting all histone deacetylase isoforms may be avoided by specifically inhibiting the one (or more) histone deacetylase isoform(s) required for inducing cell proliferation.

As previously indicated, the agent of the first aspect includes, but is not limited to, oligonucleotides and small molecule inhibitors that inhibit the activity of one or more, but less than all, HDAC isoforms. The measurement of the enzymatic activity of a histone deacetylase isoform can be achieved using known methodologies. For example, Yoshida et al. (J. Biol. Chem. 265: 17174-17179, 1990) describe the assessment of histone deacetylase enzymatic activity by the detection of acetylated histones in trichostatin A treated cells. Taunton et al. (Science 272: 408-411, 1996) similarly describes methods to measure histone deacetylase enzymatic activity using endogenous and recombinant HDAC. Both Yoshida et al. J. Biol. Chem. 265: 17174-17179, 1990) and Taunton et al. (Science 272: 408-411, 1996) are hereby incorporated by reference.

Preferably, the histone deacetylase small molecule inhibitor(s) of the invention that inhibits a histone deacetylase isoform that is required for induction of cell proliferation is a histone deacetylase small molecule inhibitor that interacts with and reduces the enzymatic activity of fewer than all histone deacetylase isoforms.

In an seventh aspect, the invention provides a method for identifying a histone deacetylase isoform that is involved in induction of cell differentiation, comprising contacting a cell with an antisense oligonucleotide that inhibits the expression of a histone deacetylase isoform, wherein induction of differentiation in the contacted cell identifies the histone deacetylase isoform as a histone deacetylase isoform that is involved in induction of cell differentiation. Preferably, the cell is a neoplastic cell. In certain preferred embodiments, the histone deacetylase isoform is HDAC-1, HDAC-2, HDAC-3, HDAC-4, HDAC-5, HDAC-6, HDAC-7, or HDAC-8.

The phrase “inducing cell differentiation” and similar terms are used to denote the ability of a histone deacetylase antisense oligonucleotide or histone deacetylase small molecule inhibitor (or combination thereof) to induce differentiation in a contacted cell as compared to a cell that is not contacted. Thus, a neoplastic cell, when contacted with a histone deacetylase antisense oligonucleotide or histone deacetylase small molecule inhibitor (or both) of the invention, may be induced to differentiate, resulting in the production of a daughter cell that is phylogenetically more advanced than the contacted cell.

In an eighth aspect, the invention provides a method for inhibiting cell proliferation in a cell, comprising contacting a cell with at least two of the reagents selected from the group consisting of an antisense oligonucleotide that inhibits a specific histone deacetylase isoform, a histone deacetylase small molecule inhibitor, an antisense oligonucleotide that inhibits a DNA methyltransferase, and a DNA methyltransferase small molecule inhibitor. In one embodiment, the inhibition of cell growth of the contacted cell is greater than the inhibition of cell growth of a cell contacted with only one of the reagents. In certain preferred embodiments, each of the reagents selected from the group is substantially pure. In preferred embodiments, the cell is a neoplastic cell. In yet additional preferred embodiments, the reagents selected from the group are operably associated.

Antisense oligonucleotides that inhibit DNA methyltransferase are described in Szyf and von Hofe, U.S. Pat. No. 5,578,716, the entire contents of which are incorporated by reference. DNA methyltransferase small molecule inhibitors include, without limitation, 5-aza-2′-deoxycytidine (5-aza-dC), 5-fluoro-2′-deoxycytidine, 5-aza-cytidine (5-aza-C), or 5,6-dihydro-5-aza-cytidine.

In a ninth aspect, the invention provides a method for modulating cell proliferation or differentiation comprising contacting a cell with an agent of the first aspect of the invention, wherein one or more, but less than all, HDAC isoforms are inhibited, which results in a modulation of proliferation or differentiation. In preferred embodiments, the cell proliferation is neoplasia.

For purposes of this aspect, it is unimportant how the specific HDAC isoform is inhibited. The present invention has provided the discovery that specific individual HDACs are involved in cell proliferation or differentiation, whereas others are not. As demonstrated in this specification, this is true regardless of how the particular HDAC isoform(s) is/are inhibited.

By the term “modulating” proliferation or differentiation is meant altering by increasing or decreasing the relative amount of proliferation or differentiation when compared to a control cell not contacted with an agent of the first aspect of the invention. Preferably, there is an increase or decrease of about 10% to 100%. More preferably, there is an increase or decrease of about 25% to 100%. Most preferably, there is an increase or decrease of about 50% to 100%. The term “about” is used herein to indicate a variance of as much as 20% over or below the stated numerical values.

In certain preferred embodiments, the histone deacetylase isoform is selected from HDAC-1, HDAC-2, HDAC-3, HDAC-4, HDAC-5, HDAC-6, HDAC-7 and HDAC-8. In certain preferred embodiments, the histone deacetylase isoform is HDAC-1.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein. Such equivalents are considered to be within the scope of this invention, and are covered by the appended claims.

### EXAMPLES

### Example 1

### Synthesis and Identification of Antisense Oligonucleotides

Antisense (AS) and mismatch (MM) oligodeoxynucleotides (oligos) were designed to be directed against the 5′- or 3′-untranslated region (UTR) of the targeted gene. Oligos were synthesized with the phosphorothioate backbone and the 4×4 nucleotides 2′-O-methyl modification on an automated synthesizer and purified by preparative reverse-phase HPLC. All oligos used were 20 base pairs in length.

To identify antisense oligodeoxynucleotide (ODN) capable of inhibiting HDAC-1 expression in human cancer cells, eleven phosphorothioate ODNs containing sequences complementary to the 5′ or 3′ UTR of the human HDAC-1 gene (GenBank Accession No. U50079) were initially screened in T24 cells at 100 nM. Cells were harvested after 24 hours of treatment, and HDAC-1 RNA expression was analyzed by Northern blot analysis. This screen identified HDAC-1 AS1 and AS2 as ODNs with antisense activity to human HDAC-1. HDAC-1 MM oligo was created as a control; compared to the antisense oligo, it has a 6-base mismatch.

Twenty-four phosphorothioate ODNs containing sequences complementary to the 5′ or 3′ UTR of the human HDAC-2 gene (GenBank Accession No. U31814) were screened as above. HDAC-2 AS was identified as an ODN with antisense activity to human HDAC-2. HDAC-2 MM was created as a control; compared to the antisense oligo, it contains a 7-base mismatch.

Twenty-one phosphorothioate ODNs containing sequences complementary to the 5′ or 3′ UTR of the human HDAC-3 gene (GenBank Accession No. AF039703) were screened as above. HDAC-3 AS was identified as an ODN with antisense activity to human HDAC-3. HDAC-3 MM oligonucleotide was created as a control; compared to the antisense oligonucleotide, it contains a 6-base mismatch.

Seventeen phosphorothioate ODNs containing sequences complementary to the 5′ or 3′ UTR of the human HDAC-4 gene (GenBank Accession No. AB006626) were screened as above. HDAC-4 AS1 and AS2 were identified as ODNs with antisense activity to human HDAC-4. HDAC-4 MM1 and MM2 oligonucleotides were created as controls; compared to the antisense oligonucleotides, they each contain a 6-base mismatch.

Thirteen phosphorothioate ODNs containing sequences complementary to the 5′ or 3′ untranslated regions of the human HDAC-5 gene (GenBank Accession No. AF039691) were screened as above. HDAC-5 AS was identified as an ODN with antisense activity to human HDAC-5.

Thirteen phosphorothioate ODNs containing sequences complementary to the 5′ or 3′ untranslated regions of the human HDAC-6 gene (GenBank Accession No. AJ011972) were screened as above. HDAC-6 AS was identified as an ODN with antisense activity to human HDAC-6. HDAC-6 MM oligo was created as a control; compared to the antisense oligo, it contains a 7-base mismatch.

Eighteen phosphorothioate ODNs containing sequences complementary to the 5′ or 3′ untranslated regions of the human HDAC-7 gene (GenBank Accession No. AF239243) were screened as above. HDAC-7 AS was identified as an ODN with antisense activity to human HDAC-7.

Fourteen phosphorothioate ODNs containing sequences complementary to the 5′ or 3′ untranslated regions of the human HDAC-8 gene (GenBank Accession No. AF230097) were screened as above. HDAC-8 AS was identified as an ODN with antisense activity to human HDAC-8.

### Example 2

### HDAC AS ODNs Specifically Inhibit Expression at the mRNA Level

In order to determine whether AS ODN treatment reduced HDAC expression at the mRNA level, human A549 cells were treated with 50 nM of antisense (AS) oligonucleotide directed against human HDAC-3 or its corresponding mismatch (MM) oligo for 48 hours, and A549 cells were treated with 50 nM or 100 nM of AS oligonucleotide directed against human HDAC-1, HDAC-2, HDAC-4, HDAC-5, HDAC-6 or HDAC-7 or the appropriate MM oligonucleotide (100 nM) for 24 hours.

Briefly, human A549 and/or T24 human bladder carcinoma cells were seeded in 10 cm tissue culture dishes one day prior to oligonucleotide treatment. The cell lines were obtained from the American Type Culture Collection (ATCC) (Manassas, Va.) and were grown under the recommended culture conditions. Before the addition of the oligonucleotides, cells were washed with PBS (phosphate buffered saline). Next, lipofectin transfection reagent (GIBCO BRL Mississauga, Ontario, Calif.), at a concentration of 6.25 μg/ml, was added to serum free OPTIMEM medium (GIBCO BRL, Rockville, Md.), which was then added to the cells. The oligonucleotides to be screened were then added directly to the cells (i.e., one oligonucleotide per plate of cells). Mismatched oligonucleotides were used as controls. The same concentration of oligonucleotide (e.g., 50 nM) was used per plate of cells for each oligonucleotide tested.

Cells were harvested, and total RNAs were analyzed by Northern blot analysis. Briefly, total RNA was extracted using RNeasy miniprep columns (QIAGEN). Ten to twenty μg of total RNA was run on a formaldehyde-containing 1% agarose gel with 0.5 M sodium phosphate (pH 7.0) as the buffer system. RNAs were then transferred to nitrocellulose membranes and hybridized with the indicated radiolabeled DNA probes. Autoradiography was performed using conventional procedures.

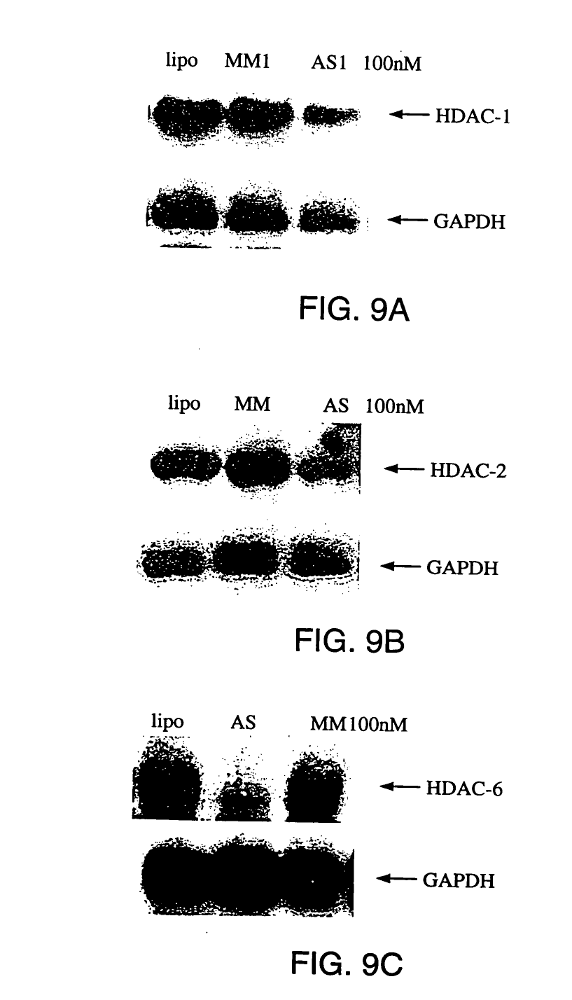

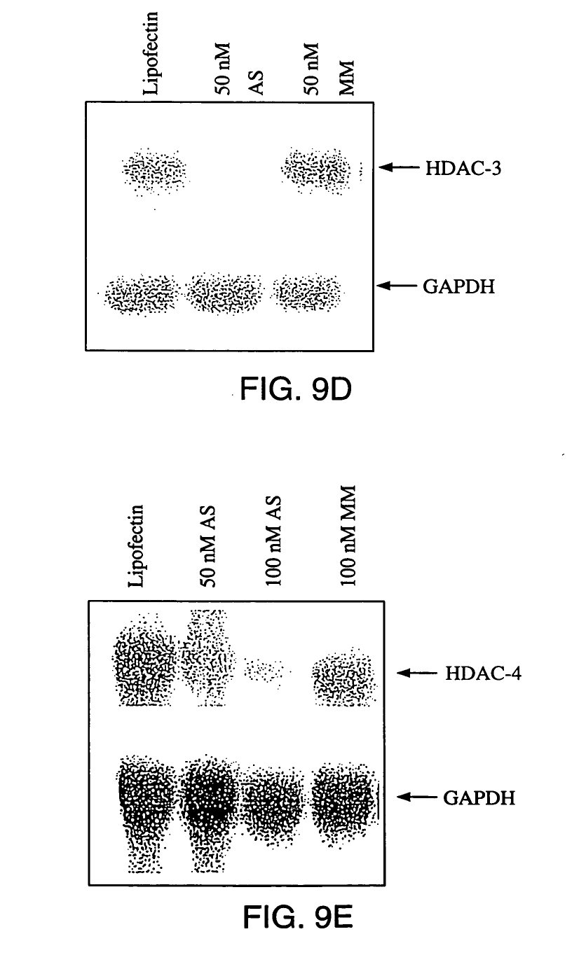

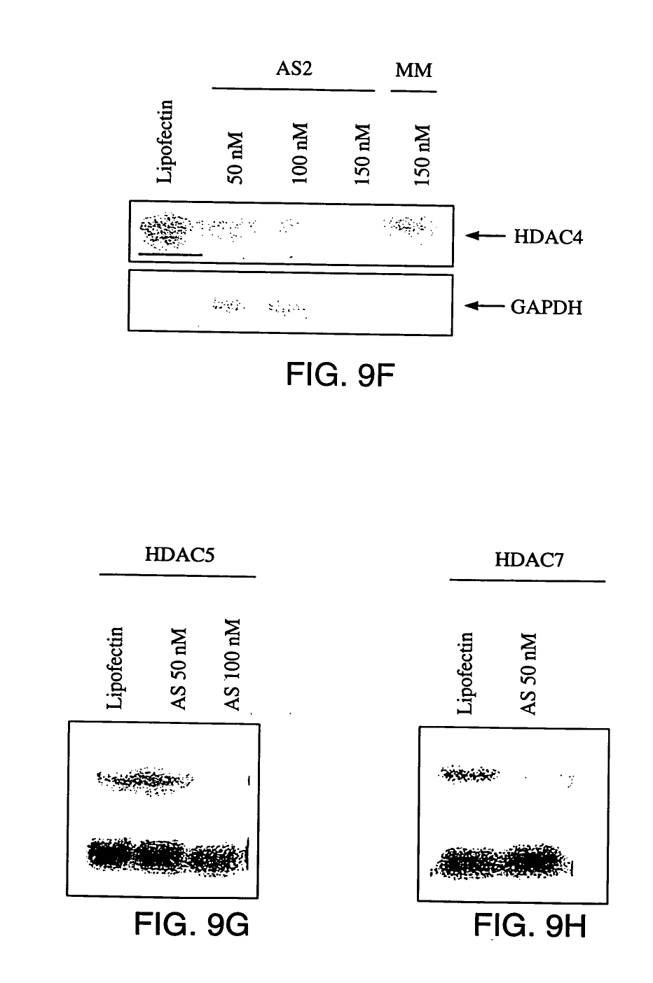

FIGS. 9A-9I present results of experiments conducted with HDAC-1 (FIG. 9A), HDAC-2 (FIG. 9B), HDAC-6 (FIG. 9C), HDAC-3 (FIG. 9D), HDAC-4 (FIGS. 9E and 9F), HDAC-5 (FIG. 9G), HDAC-7 (FIG. 9H), and HDAC-8 (FIG. 9I) AS ODNs.

Treatment of cells with the respective HDAC AS ODN significantly inhibits the expression of the targeted HDAC mRNA in human A549 cells.

### Example 3

### HDAC OSDNs Inhibit HDAC Protein Expression

In order to determine whether treatment with HDAC OSDNs would inhibit HDAC protein expression, human A549 cancer cells were treated with 50 nM of paired antisense or its mismatch oligos directed against human HDAC-1, HDAC-2, HDAC-3, HDAC-4 or HDAC-6 for 48 hours. OSDN treatment conditions were as previously described.

Cells were lysed in buffer containing 1% Triton X-100, 0.5% sodium deoxycholate, 5 mM EDTA, 25 mM Tris-HC1, pH 7.5, plus protease inhibitors. Total protein was quantified by the protein assay reagent from Bio-Rad (Hercules, Calif.). 100 ug of total protein was analyzed by SDS-PAGE. Next, total protein was transferred onto a PVDF membrane and probed with various HDAC-specific primary antibodies. Rabbit anti-HDAC-1 (H-51), anti-HDAC-2 (H-54) antibodies (Santa Cruz Biotechnologies, Santa Cruz, Calif.) were used at 1:500 dilution. Rabbit anti-HDAC-3 antibody (Sigma, St. Louis, Mo.) was used at a dilution of 1:1000. Anti-HDAC-4 antibody was prepared as previously described (Wang, S. H. et al., (1999) Mol. Cell. Biol. 19:7816-27), and was used at a dilution of 1:1000. Anti-HDAC-6 antibody was raised by immunizing rabbits with a GST fusion protein containing a fragment of HDAC-6 protein (amino acid #990 to #1216, GenBank Accession No. AAD29048). Rabbit antiserum was tested and found only to react specifically to the human HDAC-6 isoform. HDAC-6 antiserum was used at 1:500 dilution in Western blots to detect HDAC-6 in total cell lysates. Horse Radish Peroxidase conjugated secondary antibody was used at a dilution of 1:5000 to detect primary antibody binding. The secondary antibody binding was visualized by use of the Enhanced chemiluminescence (ECL) detection kit (Amersham-Pharmacia Biotech., Inc., Piscataway, N.J.).

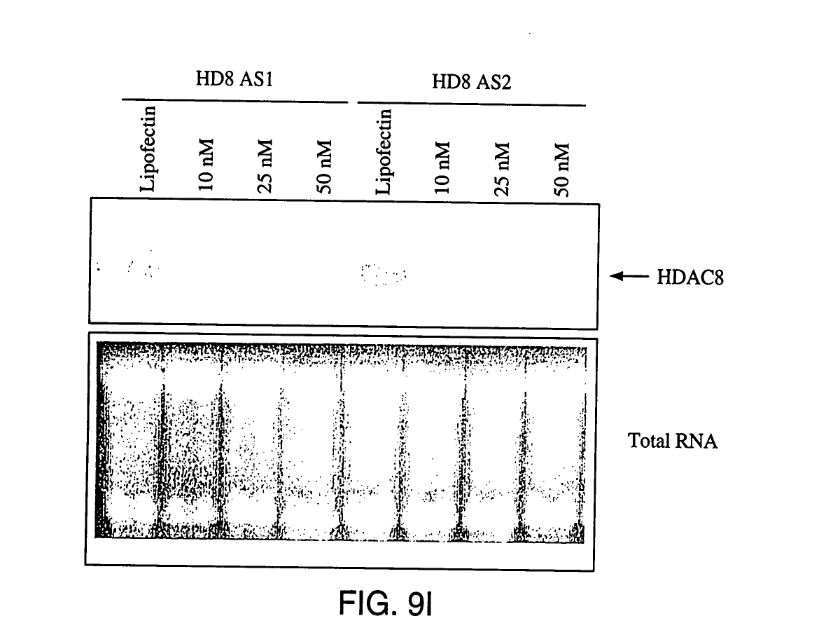

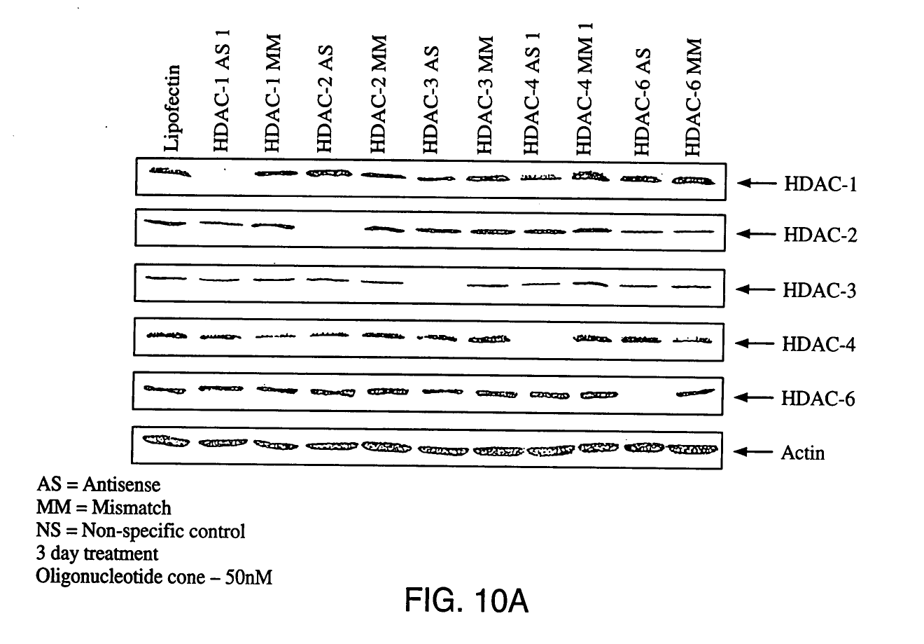

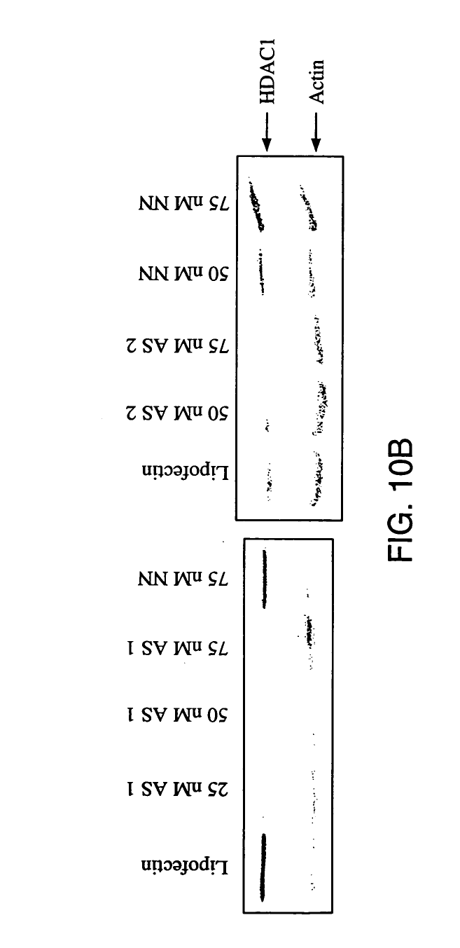

As shown in FIG. 10A, the treatment of cells with HDAC-1, HDAC-2, HDAC-3, HDAC-4 or HDAC-6 ODNs for 48 hours specifically inhibits the expression of the respective HDAC isotype protein. FIG. 10B presents dose dependent response for the inhibited expression of HDAC-1 protein in cells treated with two HDAC-1 AS ODNs. As predicted, treatment of cells with the respective mismatch (MM) control oligonucleotide does not result in a significant decrease in HDAC-1 protein expression in the treated cells.

In order to demonstrate that the level of HDAC protein expression is an important factor in the cancer cell phenotype, experiments were done to determine the level of HDAC isotype expression in normal and cancer cells. Western blot analysis was performed as described above.

The results are presented in Table 3 clearly demonstrate that HDAC-1, HDAC-2, HDAC-3, HDAC-4, and HDAC-6, isotype proteins are overexpressed in cancer cell lines.

TABLE 3
Expression Level of HDAC Isotypes in Human
Normal and Cancer Cells

| States of | Tissue | Cell | | | | | |
|---|---|---|---|---|---|---|---|
| Cell | Type | Designation | HDAC-1 | HDAC-2 | HDAC-3 | HDAC-4 | HDAC-6 |
| Normal | Breast | HMEC | − | + | ++ | + | + |
| | Epithelial | | | | | | |
| Normal | Foreskin | MRHF | − | + | + | ++ | + |
| | Fibroblasts | | | | | | |
| Cancer | Bladder | T24 | +++ | ++ | +++ | ++ | +++ |
| Cancer | Lung | A549 | ++ | +++ | +++ | +++ | ++ |
| Cancer | Colon | SW48 | +++ | +++ | +++ | +++ | +++ |
| Cancer | Colon | HCT116 | ++++ | +++ | +++ | ++++ | +++ |
| Cancer | Colon | HT29 | +++ | +++ | +++ | +++ | +++ |
| Cancer | Colon | NCI-H446 | ++ | ++++ | +++ | ++++ | ++ |
| Cancer | Cervix | Hela | +++ | ++++ | +++ | +++ | +++ |
| Cancer | Prostate | DU145 | +++ | +++ | +++ | ++++ | +++ |
| Cancer | Breast | MDA-MB- | ++ | +++ | +++ | +++ | ++++ |
| | | 231 | | | | | |
| Cancer | Breast | MCF-7 | +++ | +++ | +++ | ++ | ++ |
| Cancer | Breast | T47D | +++ | +++ | +++ | ++ | +++ |
| Cancer | Kidney | 293T | +++ | ++++ | ++++ | ++ | ++ |
| Cancer | Leukemia | K562 | +++ | ++++ | ++++ | ++++ | +++ |
| Cancer | Leukemia | Jurkat T | +++ | ++ | ++++ | ++ | ++ |

(−): not detectable;
(+): detectable;
(++): 2X over (+);
(+++): 5X over (+);
(++++): 10x over (+)

### Example 4

### Effect of HDAC Isotype Specific OSDNs on Cell Growth and Apoptosis

In order to determine the effect of HDAC OSDNs on cell growth and cell death through apoptosis, A549 or T24 cells, MDAmb231 cells, and HMEC cells (ATCC, Manassas, Va.) were treated with HDAC OSDNs as previously described.

For the apoptosis study, cells were analyzed using the Cell Death Detection ELISA^{Plus} kit (Roche Diagnostic GmBH, Mannheim, Germany) according to the manufacturer's directions. Typically, 10,000 cells were plated in 96-well tissue culture dishes for 2 hours before harvest and lysis. Each sample was analyzed in duplicate. ELISA reading was done using a MR700 plate reader (DYNEX Technology, Ashford, Middlesex, England) at 410 nm. The reference was set at 490 nm.

For the cell growth analysis, human cancer or normal cells were treated with 50 nM of paired AS or MM oligos directed against human HDAC-1, HDAC-2, HDAC-3, HDAC-4 or HDAC-6 for 72 hours. Cells were harvested and cell numbers counted by trypan blue exclusion using a hemocytometer. Percentage of inhibition was calculated as (100—AS cell numbers/control cell numbers)%.

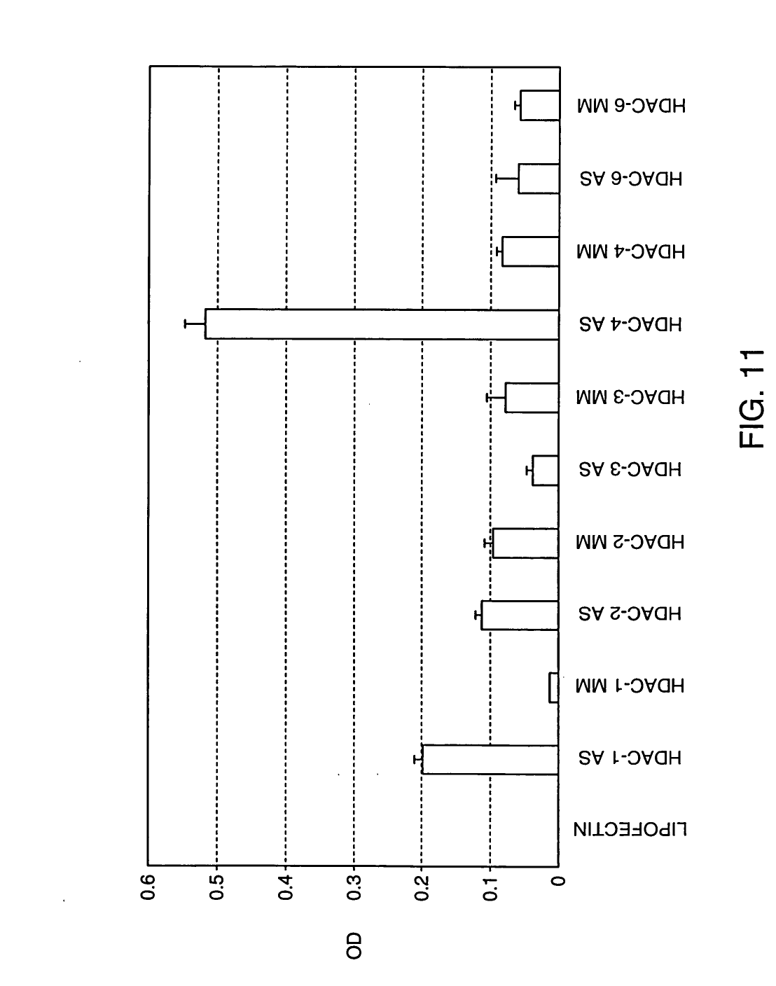

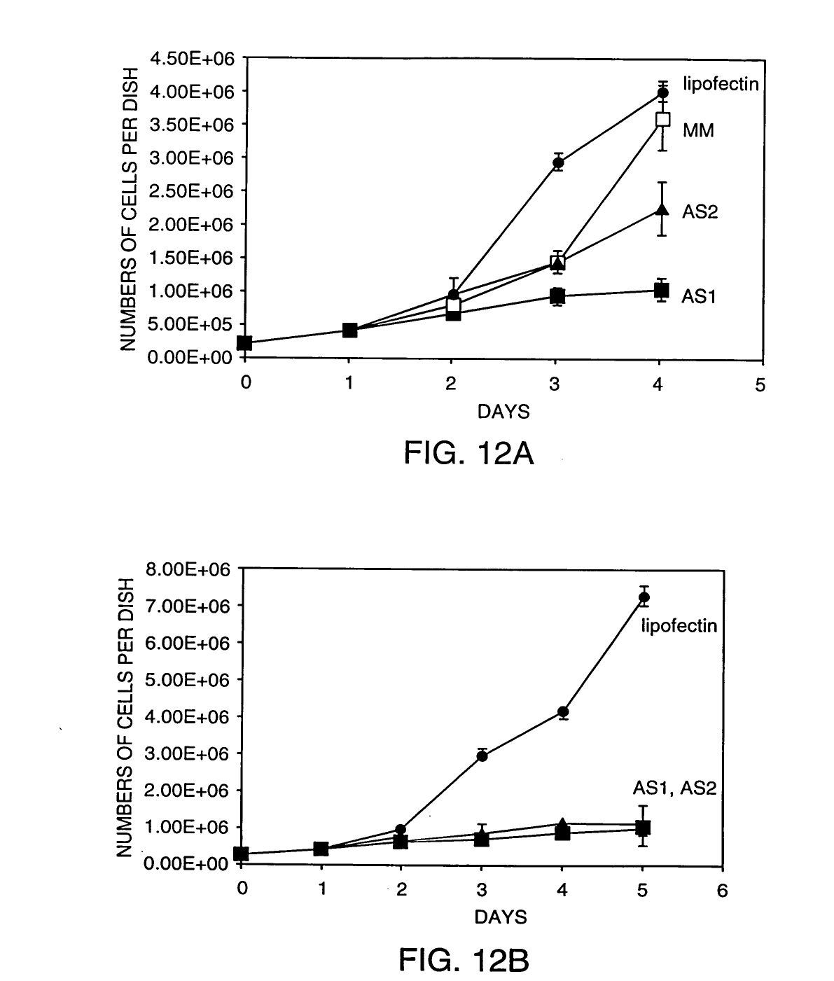

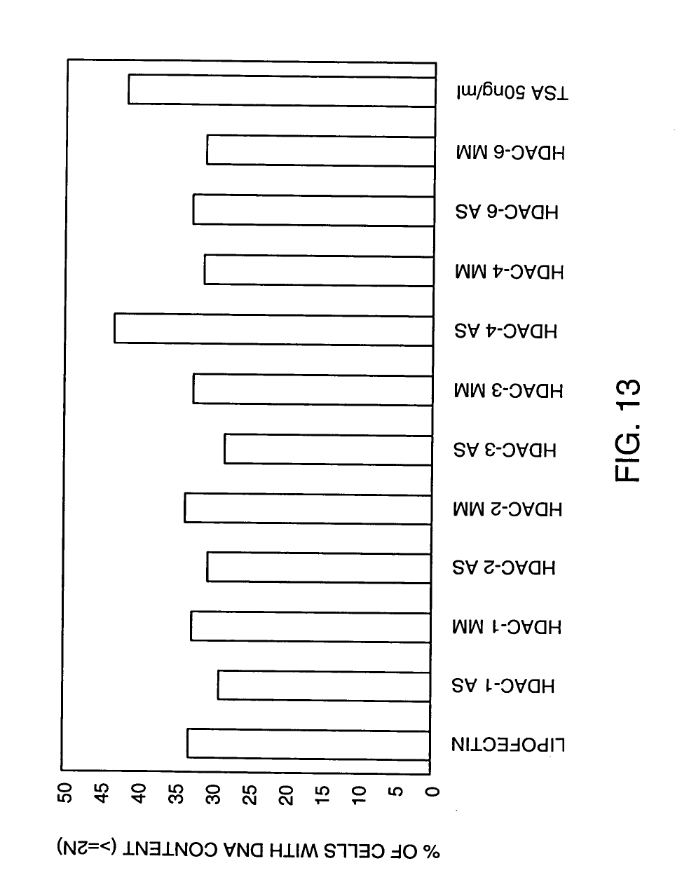

Results of the study are shown in FIGS. 11-13, and in Table 4 and Table 5. Treatment of human cancer cells by HDAC-4 AS, and to a lesser extent, HDAC 1 AS, induces growth arrest and apoptosis of various human cancer. The corresponding mismatches have no effect. The effects of HDAC-4 AS or HDAC-1 AS on growth inhibition and apoptosis are significantly reduced in human normal cells. In contrast to the effects of HDAC-4 or HDAC-1 AS oligos, treatment with human HDAC-3 and HDAC-6 OSDNs has no effect on cancer cell growth or apoptosis, and treatment with human HDAC-2 OSDN has a minimal effect on cancer cell growth inhibition. Since T24 cells are p53 null and A549 cells have functional p53 protein, this induction of apoptosis is independent of p53 activity.

TABLE 4
Effect of HDAC Isotype-Specific OSDNs on Human Normal
and Cancer Cells Growth Inhibition (AS vs. MM)

| | Cancer | Normal | | |
|---|---|---|---|---|
| | Cells | Cells | | |
| | A549 | T24 | MDAmb231 | HMEC |
| HDAC-1 AS1 | ++(+) | +(+) | +/− | +/− |
| HDAC-2 AS | +(+) | +/− | − | +/− |
| HDAC-3 AS | − | − | − | − |
| HDAC-4 AS1 | +++ | ++ | ++ | +/− |
| HDAC-6 AS | − | − | +/− | − |

“−”: no inhibition,
“+”: <50% inhibition,
“++”: 50-75% inhibition,
“+++”: >75% inhibition

TABLE 5
Effect of HDAC Isotype-Specific OSDNs on Human Normal
and Cancer Cells Apoptosis After 48 Hour Treatment

| | A549 | T24 | MDAmb231 | HMEC |
|---|---|---|---|---|
| HDAC-1 AS1 | + | − | | − |
| HDAC-2 AS | − | − | − | − |
| HDAC-3 AS | − | − | − | − |
| HDAC-4 AS1 | +++ | + | ++ | − |
| HDAC-6 AS | − | − | − | − |
| TSA (100 ng/ml) | ++ | ++ | ++ | + |

“−”: < = 2x fold over non-specific background;
“+”: 2-3X fold;
“++”: 3-5X fold;
“+++”: 5-8X fold;
“++++”: 8X fold

### Example 5

### Inhibition of HDAC Isotypes Induces the Expression of Growth Regulatory Genes

In order to understand the mechanism of growth arrest and apoptosis of cancer cells induced by HDAC-1 or HDAC-4 AS treatment, RNase protection assays were used to analyze the mRNA expression of cell growth regulators (p21 and GADD45) and proapoptotic gene Bax.

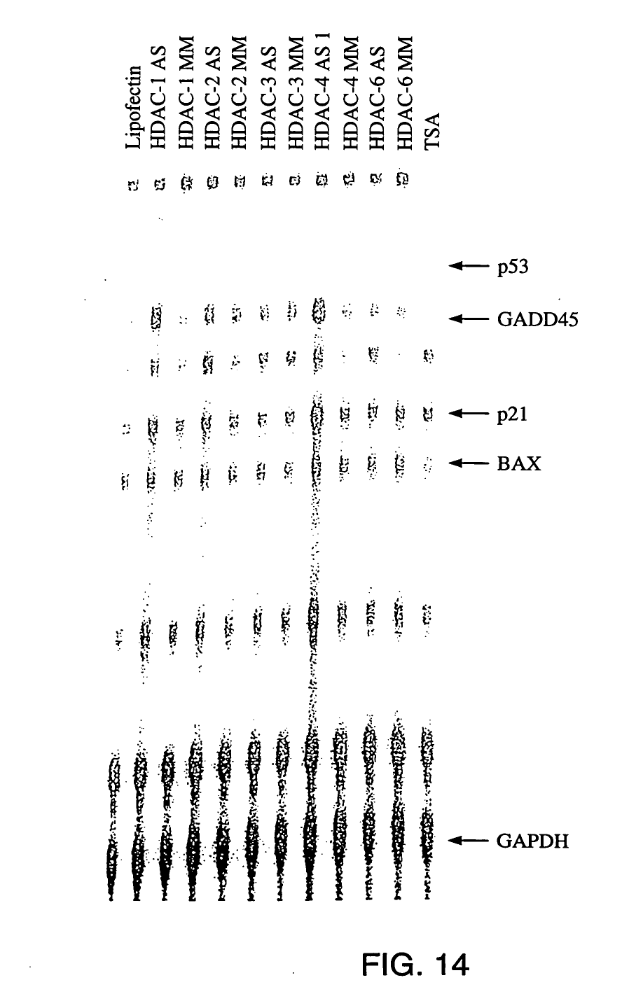

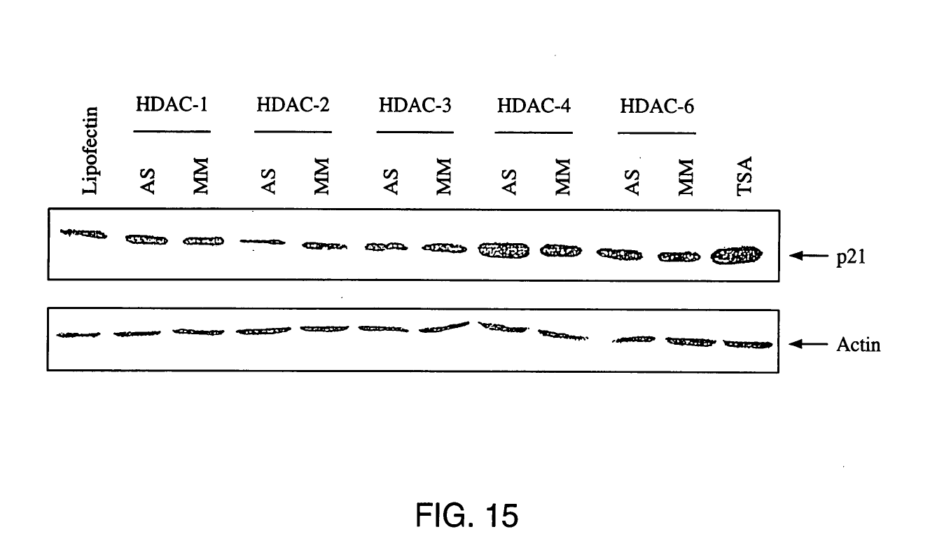

Briefly, human cancer A549 or T24 cells were treated with HDAC isotype-specific antisense oligonucleotides (each 50 nM) for 48 hours. Total RNAs were extracted and RNase protection assays were performed to analyzed the mRNA expression level of p21 and GADD45. As a control, A549 cells were treated by lipofectin with or without TSA (250 ng/ml) treatment for 16 hours. These RNase protection assays were done according to the following procedure. Total RNA from cells was prepared using “RNeasy miniprep kit” from QIAGEN following the manufacturer's manual. Labeled probes used in the protection assays were synthesized using “hStress-1 multiple-probe template sets” from Pharmingen (San Diego, Calif., U.S.A.) according to the manufacturer's instructions. Protection procedures were performed using “RPA II™ Ribonuclease Protection Assay Kit” from Ambion, (Austin, Tex.) following the manufacturer's instructions. Quantitation of the bands from autoradiograms was done by using Cyclone™ Phosphor System (Packard Instruments Co. Inc., Meriden, Conn.). The results are shown in FIGS. 14, 15 and Table 6.

TABLE 6
Up-Regulation of p21, GADD45 and Bax After Cell
Treatment with Human HDAC Isotype-Specific Antisenses

| | A549 | | | T24 | | |
|---|---|---|---|---|---|---|
| | p21 | GADD45 | Bax | p21 | GADD45 | Bax |
| HDAC-1 | 17 | 5.0 | 0.8 | 2.4 | 3.4 | 0.9 |
| HDAC-2 | 1.1 | 1.2 | 1.0 | 1.0 | 1.0 | 0.9 |
| HDAC-3 | 0.7 | 0.9 | 1.0 | 0.9 | 1.0 | 1.0 |
| HDAC-4 | 3.1 | 5.7 | 2.6 | 2.8 | 2.7 | 1.9 |
| HDAC-6 | 1.0 | 1.0 | 1.0 | 1.0 | 0.8 | 1.1 |
| TSA vs lipofectin | 2.8 | 0.6 | 0.8 | | | |

Values indicate the fold induction of transcription as measured by RNase protection analysis for the respective AS vs. MM HDAC isotype-specific oligos.

Results of the experiments are presented in Table 6. The inhibition of HDAC-4 in both A549 and T24 cancer cells dramatically up-regulates both p21 and GADD45 expression. Inhibition of HDAC-1 by antisense oligonucleotides induces p21 expression but more greatly induces GADD45 expression. Inhibition of HDAC-4, upregulates Bax expression in both A549 and T24 cells. The effect of HDAC-4 AS treatment (50 nM, 48 hrs) on p21 induction in A549 cells is comparable to that of TSA (0.3 to 0.8 uM, 16 hrs).

Experiments were also conducted to examine the affect of HDAC antisense oligonucleotides on HDAC protein expression. In A549 cells, treatment with HDAC-4 antisene oligonucleotides results in a dramatic increase in the level of p21 protein (FIG. 15).

### Example 6

### Cyclin Gene Expression is Repressed by HDAC-1 AS Treatment

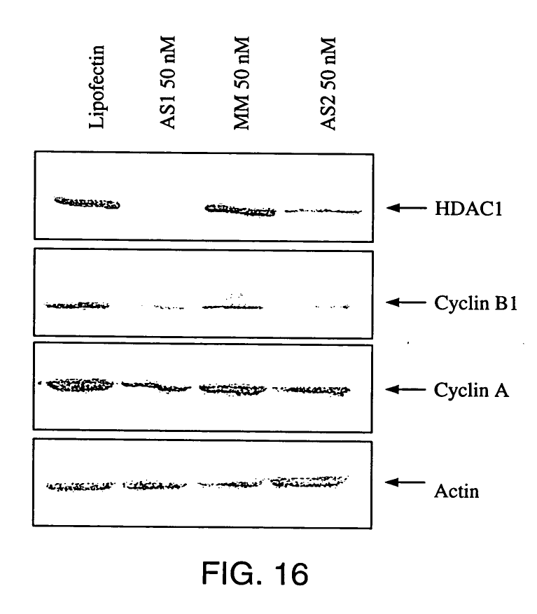

Human cancer A549 cells were treated with AS1, AS2 or MM oligo directed human HDAC1 for 48 hours. Total cell lysates were harvested and analyzed by Western blot using antibodies against human HDAC1, cyclin B1, cyclin A and actin (all from Santa Cruz Biotechnology, Inc., Santa Cruz, Calif.). AS1 or AS2 both repress expression of cyclin B1 and A. Downregulation of cyclin A and B1 expression by AS1 and AS2 correlates well with their ability to inhibit cancer cell growth. (FIG. 16)

### Example 7

### Inhibition of Growth in Soft Agar

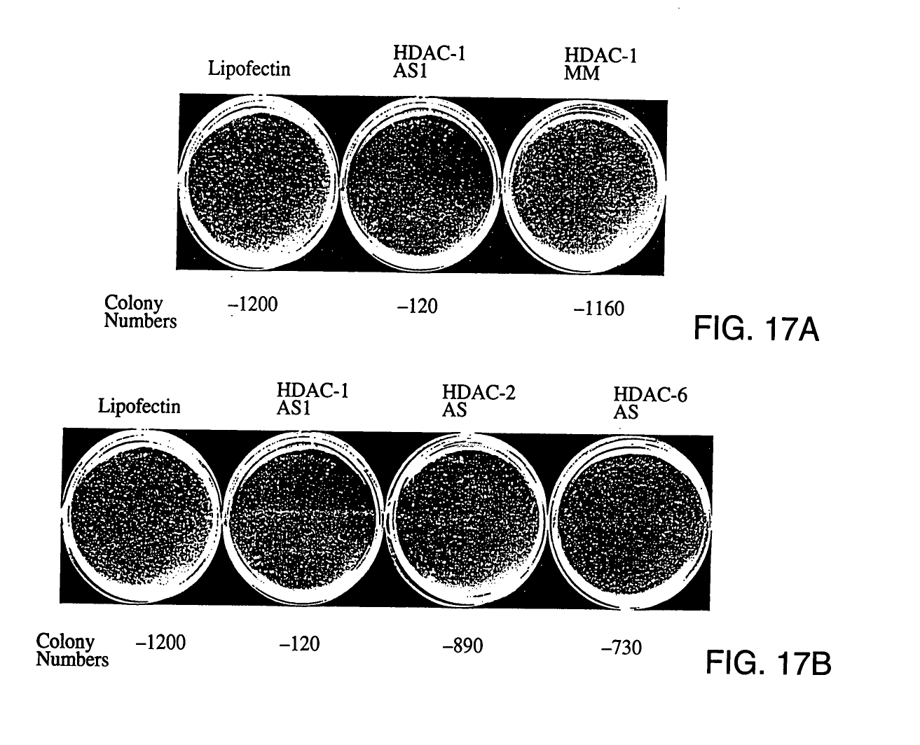

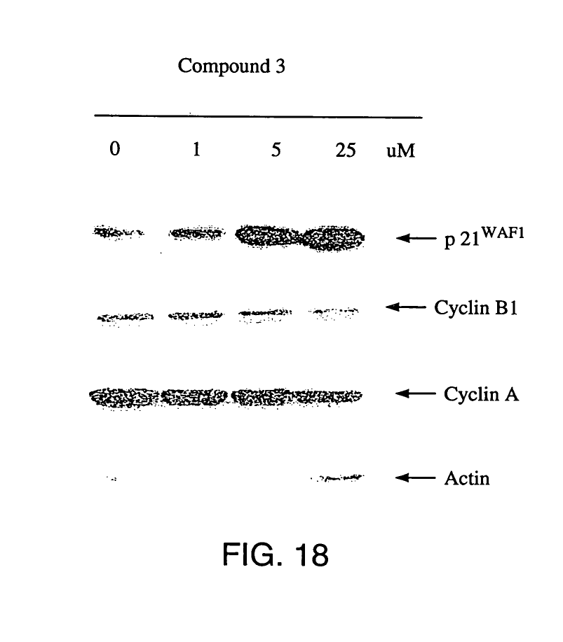

1.3 g granulated agar (DIDFCO) was added to 100 ml deionized water and boiled in a microwave to sterilize. The boiled agar was held at 55●C until further use. Iscove's Modified Dulbecco's Medium (GIBCO/BRL), 100× Penicillin-Streptomycin-Glutamine (GIBCO/BRL) and fetal bovine serum (medicorp) were pre-warmed at 37●C. To 50 ml sterile tubes was added 9 ml Isove's medium, 2 ml fetal bovine serum and 0.2 ml 100× Pen-Strep-Gln. Then 9 ml 55●C 1.3% agar was added to each tube. The tube contents were mixed immediately, avoiding air bubbles, and 2.5 ml of the mixture was poured into each sterile 6 cm petri dish to form a polymerized bottom layer. Dishes with polymerized bottom layers were then put in a CO2 incubator at 37●C until further use. In 50 ml sterile tubes were prewarmed at 37●C for each 4 cell lines/samples, 20 ml Iscove's medium, 0.4 ml 100× Pen-Strp-Gln and 8 ml fetal bovine serum. Cells were trypsinized and counted by trypan blue staining and 20,000 cells were aliquotted into a sterile 15 ml tube. To the tube was then added DMEM with low glucose (GIBCO/BRL) +10% fetal bovine serum +Pen-Strep-Gln to a final volume of 1 ml. To the prewarmed 37●C mix in the 50 ml tube was quickly added 8 ml 55●C 1.3% agar, which was then mixed well. Nine ml of this mixture was then aliquotted to each 1 ml cells in the 15 ml tube which is then mixed and 5 ml aliquotted onto the ploymerized bottom layer of the 6 cm culture plates and allowed to polymerize at room temperature. After polymerization, 2.5 ml bottom layer mix was gently added over the cell layer. Plates were wrapped up in foil paper and incubated in a CO2 incubator at 37° ●C for three weeks, at which time colonies in agar are counted. The results are shown in FIG. 17.

These results demonstrate that an antisense oligonucleotide complementary to HDAC-1 inhibits growth of A549 cells in soft agar, but antisense oligonucleotides complementary to HDAC-2 or HDAC-6, or mismatch controls, do not.

### Example 8

### Inhibition of HDAC Isotypes by Small Molecules

In order to demonstrate the identification of HDAC small molecule inhibitors, HDAC small molecule inhibitors were screened in histone deacetylase enzyme assays using various human histone deacetylase isotypic enzymes (i.e., HDAC-1, HDAC-3, HDAC-4 and HDAC-6). Cloned recombinant human HDAC-1, HDAC-3 and HDAC-6 enzymes, which were tagged with the Flag epitope (Grozinger, C. M., et al., Proc. Natl. Acad. Sci. U.S.A. 96:4868-4873 (1999)) in their C-termini, were produced by a baculovirus expression system in insect cells.

Flag-tagged human HDAC-4 enzyme was produced in human embronic kidney 293 cells after transformation by the calcium phosphate precipitation method. Briefly, 293 cells were cultured in Dulbecco's Modified Eagle Medium (DMEM) containing 10% fetal bovine serum and antibiotics. Plasmid DNA encoding Flag-tagged human HDAC-4 was precipitated by ethanol and resuspend in sterile water. DNA-calcium precipitates, formed by mixing DNA, calcium choloride and 2× HEPES-buffered saline solution, were left on 293 cells for 12-16 hours. Cells were return to serum-contained. DMEM medium and harvested at 48 hour post transfection for purification of Flag-tagged HDAC-4 enzyme.

HDAC-1 and HDAC-6 were purified on a Q-Sepharose column, followed by an anti-Flag epitope affinity column. The other HDAC isotypes, HDAC-3 and HDAC-4, were purified directly on an anti-Flag affinity column.

For the deacetylase assay, 20,000 cpm of an [³H]-metabolically-labeled acetylated histone was used as a substrate. Histones were incubated with cloned recombinant human HDAC enzymes at 37° C. For the HDAC-1 asasy, the incubation time was 10 minutes, and for the HDAC-3, HDAC-4 and HDAC-6 assays, the incubation time was 2 hours. All assay conditions were pre-determined to be certain that each reaction was linear. Reactions were stopped by adding acetic acid (0.04 M, final concentration) and HCl (250 mM, final concentration). The mixture was extracted with ethyl acetate, and the released [³H]-acetic acid was quantified by liquid scintillation counting. For the inhibition studies, HDAC enzyme was preincubated with test compounds for 30 minutes at 4° C. prior to the start of the enzymatic assay. IC₅₀ values for HDAC enzyme inhibitors were identified with dose response curves for each individual compound and, thereby, obtaining a value for the concentration of inhibitor that produced fifty percent of the maximal inhibition.

### Example 9

### Inhibition of HDAC Activity in Whole Cells by Small Molecules

T24 human bladder cancer cells (ATCC, Manassas, Va.) growing in culture were incubated with test compounds for 16 hours. Histones were extracted from the cells by standard procedures (see e.g. Yoshida et al., supra) after the culture period. Twenty μg total core histone protein was loaded onto SDS/PAGE and transferred to nitrocellulose membranes, which were then reacted with polyclonal antibody specific for acetylated histone H-4 (Upstate Biotech Inc., Lake Placid, Wyo.). Horse Radish Peroxidase conjugated secondary antibody was used at a dilution of 1:5000 to detect primary antibody binding. The secondary antibody binding was visualized by use of the Enhanced chemiluminescence (ECL) detection kit (Amersham-Pharmacia Biotech., Inc., Piscataway, N.J.). After exposure to film, acetylated H-4 signal was quantitated by densitometry.

The results, shown in Table 2 above, demonstrate that small molecule inhibitors selective for HDAC-1 and/or HDAC-4 can inhibit histone deacetylation in whole cells.

### Example 10

### Inhibition of Cancer Cell Growth by HDAC Small Molecule Inhibitors

Two thousand (2,000) human colon cancer HCT116 cells (ATCC, Manassas, Va. were used in an MTT (3-[4,5-dimethylthiazol-2-yl]-2,5 diphenyl tetrazolium bromide) assay to quantitatively determine cell proliferation and cytotoxicity. Typically, HCT116 cells were plated into each well of the 96-well tissue culture plate and left overnight to attach to the plate. Compounds at various concentrations were added into the culture media (final DMSO concentration 1%) and incubated for 72 hours. MTT solution (obtained from Sigma as powder) was added and incubated with the cells for 4 hours at 37° C. in incubator with 5% CO₂. During the incubation, viable cells convert MTT to a water-insoluble formazan dye. Solubilizing buffer (50% N,N-dimethylformamide, 20% SDS, pH 4.7) was added to cells and incubated for overnight at 37C in incubator with 5% CO₂. Solubilized dye was quantitated by calorimetric reading at 570 nM using a reference of 630 nM. Optical density values were converted to cell number values by comparison to a standard growth curve for each cell line. The concentration test compound that reduces the total cell number to 50% that of the control treatment, i.e., 1% DMSO, is taken as the EC₅₀ value.

The results, shown in Table 2 above, demonstrate that small molecule inhibitors selective for HDAC-1 and/or HDAC-4 can affect cell proliferation.

### Example 11

### Inhibition by Small Molecules of Tumor Growth in a Mouse Model

Female BALB/c nude mice were obtained from Charles River Laboratories (Charles River, N.Y.) and used at age 8-10 weeks. Human prostate tumor cells (DU145, 2×10⁶) or human colon cancer cells (HCT116; 2×10⁶) or small lung core A549 2×106 were injected subcutaneously in the animal's flank and allowed to form solid tumors. Tumor fragments were serially passaged a minimum of three times, then approximately 30 mg tumor fragments were implanted subcutaneously through a small surgical incision under general anaesthesia. Small molecule inhibitor administration by intraperotineal or oral administration was initiated when the tumors reached a volume of 100 mm³. For intraperotineal administration, small molecule inhibitors of HDAC (40-50 mg/kg body weight/day) were dissolved in 100% DMSO and administered daily intraperitoneally by injection. For oral administration, small molecule inhibitors of HDAC (40-50 mg/kg body weight/days) were dissolved in a solution containing 65% polyethylene glycol 400 (PEG 400 (Sigma-Aldridge, Mississauga, Ontario, CA, Catalogue No. P-3265), 5% ethanol, and 30% water. Tumor volumes were monitored twice weekly up to 20 days. Each experimental group contained at least 6-8 animals. Percentage inhibition was calculated using volume of tumor from vehicle-treated mice as controls.

The results, shown in Table 2 above, demonstrate that small molecule inhibitors selective for HDAC-1 and/or HDAC-4 can inhibit the growth of tumor cells in vivo.

### Example 12

### Upregulation of p21 Expression and Down Regulation of Cyclin Gene Expression Following Treatment with Small Molecule Inhibitor

Sulfonamide aniline (compound 3, Table 2) is a small molecule HDAC1 specific inhibitor. Human HCT116 cells were treated with escalating doses of compound 3 for 16 hours. Total cell lysates were harvested and expression of p21 ^{WAF1}, cyclin B1, cyclin A and actin was analyzed by Western blot. Ariti-p21 ^{WAF1} antibody was purchased from BD Transduction Laboratories (BD Pharmingen Canada, Missasagua, Ontario). Compound 3 clearty upregulates expression of p21 ^{WAF1} and represses the expression of cyclin A and B1. The expression profile of these cell cycle regulators correlates well with the ability of compound 3 to inhibit HCT116 proliferation in MTT assays (see Table 2),

### Example 13

### Cell Cycle Arrest Induced by HDAC Small Molecule Inhibtiors

Human cancer HCT116 cells were plated at 2×10⁵ per 10-cm dish and were left to attach to the dish overnight in the incubator. Cells were treated with small molecule inhibitors at various concentrations (1 uM and 10 uM, typically, dissolved in DMSO) for 16 hours. Cells were harvested by trypsinization and washed once in 1×PBS (phosphate buffered saline). The cells were resuspended in about 200 ul 1×PBS and were fixed by slowly adding 1 ml 70% ethanol at −20° C. and were left at least overnight at −20° C. Fixed cells were centrifuged at low speed (1,000 rpm) for 5 minutes, and the cell pellets were washed again with 1×PBS. Nucleic acids from fixed cells were incubated in a staining solution (0.1% (w/v) glucose in 1×PBS containing 50 ug/ml propidium iodide) (Sigma-Aldridge, Mississauga, Ontario, CA) and RNase A (final 100 units/ml, (Sigma-Aldridge, Mississauga, Ontario, CA) for at least 30 minutes in the dark at 25° C. DNA content was measured by using a fluorescence-activated cell sorter (FACS) machine. Treatment of cells with all HDAC small molecule inhibitors in Table 2 results in a significant accumulation of cancer cell in G2/M phase of the cell cycle and concomitantly reduce the accumulation of cancer cells in S phase of the cell cycle. The ratio of cells in G2/M phase vs. cells in the S. phase was determined. The Effective concentration (EC) of a small molecule inhibitor to induce a (G2+M)/S ratio of 2.5 is calculated, as shown in Table 2.

### Example: 14

### Synthesis of Small Molecule Compound No. 2

The following provides a synthesis scheme for small molecule Compound No. 2 from Table 2.

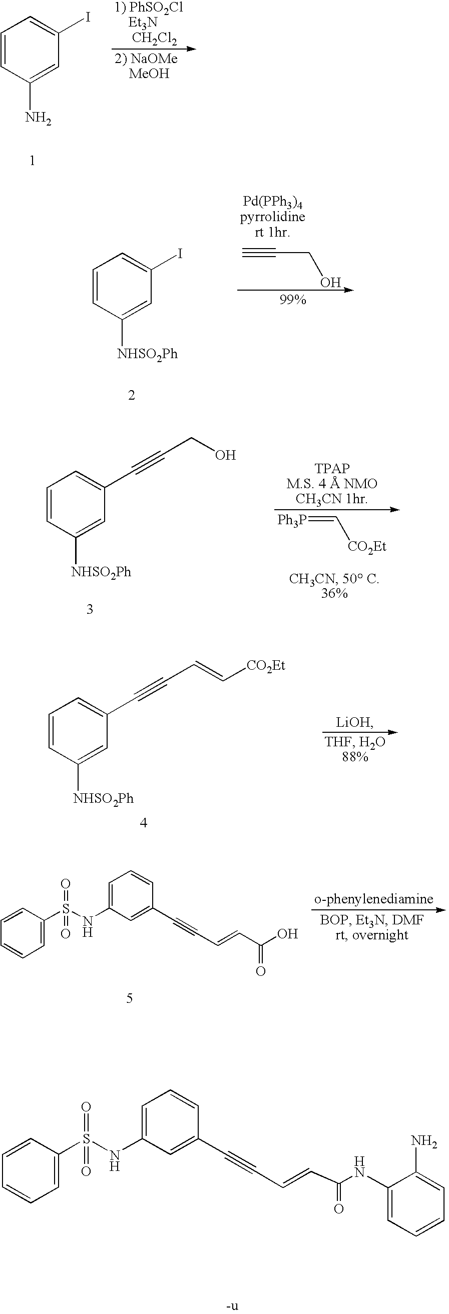

Step 1: 3-(benzenesulfonylamino)-phenyl iodide (2)

To a solution of 3-iodoaniline (5 g, 22.8 mmol), in CH₂Cl₂ (100 mL), were added at room temperature Et₃N (6.97 mL) followed by benzenesulfonyl chloride (5.84 mL). The mixture was stirred 4 h then a white precipitate was formed. A saturated aqueous solution of NaHCO₃ was added and the phases were separated. The aqueous layer was extracted several times with CH₂Cl₂ and the combined extracts were dried over (MgSO₄) then evaporated. The crude mixture was dissolved in MeOH (100 mL) and NaOMe (6 g), was added and the mixture was heated 1 h at 60° C. The solution became clear with time and HCl (1N) was added. The solvent was evaporated under reduced pressure then the aqueous phase was extracted several times with CH₂Cl₂. The combined organic extracts were dried over (MgSO₄) and evaporated. The crude material was purified by flash chromatography using (100% CH₂Cl₂) as solvent yielding the title compound 21 (7.68g, 94%) as yellow solid.

¹H NMR: (300 MHz, CDCl₃): δ 7.82-7.78 (m, 2H), 7.60-7.55 (m, 1H), 7.50-7.42 (m, 4H), 7.10-7.06 (m, 1H), 6.96 (t, J=8 Hz, 1H), 6.87 (broad s, 1H).

Step 2: 3-(benzenesulfonylamino)-phenyl-propargylic alcohol (3)

To a solution of 2 (500 mg, 1.39 mmol) in pyrrolidine (5 mL) at room temperature was added Pd(PPh₃)₄ (80 mg, 0.069 mmol), followed by CuI (26 mg, 0.139 mmol). The mixture was stirred until complete dissolution. Propargylic alcohol (162●L, 2.78 mmol) was added and stirred 6 h at room temperature. Then the solution was treated with a saturated aqueous solution of NH₄Cl and extracted several times with AcOEt. The combined organic extracts were dried over (MgSO₄) then evaporated. The residue was purified by flash chromatography using hexane/AcOEt (1:1) as solvent mixture yielding 3 (395 mg, 99%) as yellow solid.

hu 1H NMR: (300 MHz, CDCl₃): δ 7.79-7.76 (m, 2H), 7.55-7.52 (m, 1H), 7.45 (t, J=8 Hz, 2H), 7.19-7.15 (m, 3H), 7.07-7.03 (m, 1H), 4.47 (s, 2H).

Step 3: 5-[3-(benzenesulfonylamino)-phenyl]-4-yn-2-pentenoate (4)

To a solution of 3 (2.75 g, 9.58 mmol) in CH₃CN (150 mL) at room temperature were added 4-methylmorpholine N-oxide (NMO, 1.68 g, 14.37 mmol) followed by tetrapropylammonium perruthenate (TPAP, 336 mg, 0.958 mmol). The mixture was stirred at room temperature 3 h, and then filtrated through a Celite pad with a fritted glass funnel. To the filtrate carbethoxymethylenetriphenyl-phosphorane (6.66 g, 19.16 mmol) was added and the resulting solution was stirred 3 h at room temperature. The solvent was evaporated and the residue was dissolved in CH₂Cl₂ and washed with a saturated aqueous solution of NH₄Cl. The aqueous layer was extracted several times with CH₂Cl₂ then the combined organic extract were dried over (MgSO₄) and evaporated. The crude material was purified by flash chromatography using hexane/AcOEt (1:1) as solvent mixture giving 4 (1.21 g, 36%) as yellow oil.

¹H NMR: (300 MHz, CDCl₃): δ 7.81 (d, J=8 Hz, 2H), 7.56-7.43 (m, 3H), 7.26-7.21 (m, 3H), 7.13-7.11 (m, 1H), 6.93 (d, J=16 Hz, 1H), 6.29 (d, J=16 Hz, 1H), 4.24 (q, J=7 Hz, 2H), 1.31 (t, J=7 Hz, 3H).

Step 4: 5-[3-(benzenesulfonylamino)-phenyl]-4-yn-2-pentenic acid (5)

To a solution of 4 (888 mg, 2.50 mmol) in a solvent mixture of THF (10 mL) and water (10 mL) at room temperature was added LiOH (1.04 g, 25.01 mmol). The resulting mixture was heated 2 h at 60° C. and treated with HCl (1N) until pH 2. The phases were separated and the aqueous layer was extracted several times with AcOEt. The combined organic extracts were dried over (MgSO₄) then evaporated. The crude residue was purified by flash chromatography using CH₂Cl₂/MeOH (9:1) as solvent mixture yielding 5 (712 mg, 88%), as white solid.

¹H NMR: (300 MHz, DMSO-d₆): δ 7.78-7.76 (m, 2H), 7.75-7.53 (m, 3H), 7.33-7.27 (m, 1H), 7.19-7.16 (m, 3H), 6.89 (d, J=16 Hz, 1H), 6.33 (d, J=16 Hz, 1H).

Step 5: Compound 2

Coupling of 5 with o-phenylenediamine in the presence of benzotriazol-1-yloxytris(dimethylamino)phosphonium hexafluorophosphate (BOP) afforded the anilide Compound 2.

¹H NMR: (300 MHz, DMSO d₆): δ 7.77 (broad s, 4H); 7.57 (d, 1H, J=15.7 Hz); 7.35 (d, 1H, J=6.9 Hz); 7.03-6.94 (m, 6H); 6.76 (d, 1H, J=7.1 Hz); 6.59 (d, 1H, J=6.9 Hz); 4.98 (broad s, 2H); 2.19 (s, 3H).

¹³C NMR: (75 MHz, DMSO d₆): δ 162.9; 141.6; 139.8; 139.0; 137.6; 134.8; 133.6; 129.6; 128.1; 127.3; 125.9; 125.4; 124.7; 123.2; 120.7; 116.2; 115.9; 20.3.

### Example: 15

### Synthesis of Small Molecule Compound No. 3

The following provides a synthesis scheme for Compound No. 3 from Table 2.

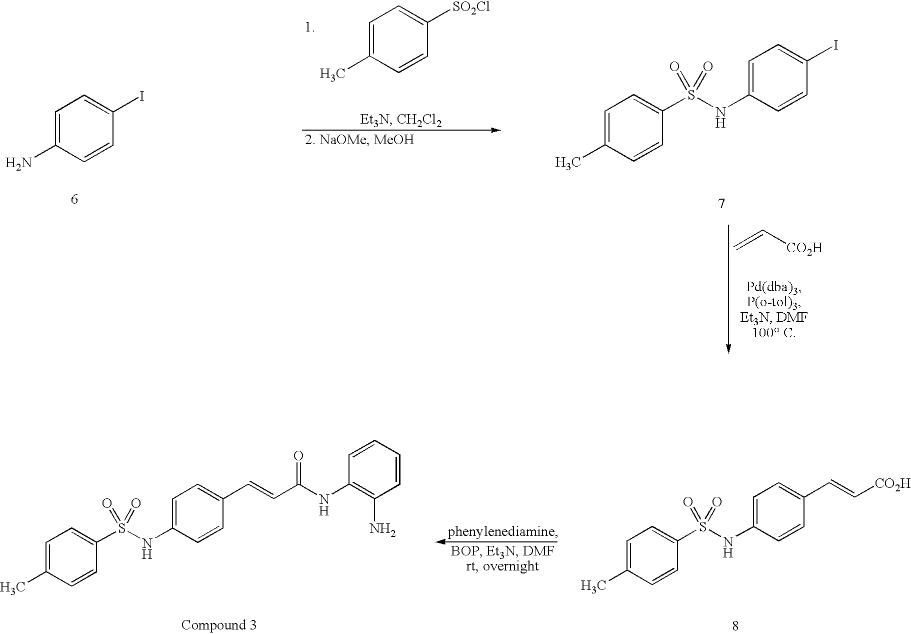

Step 1: 3-[4-(toluenesulfonylamino)-phenyl]-2-propenoic acid (8)

To a solution of 7 (1.39 mmol), in DMF (10 mL) at room temperature were added tris(dibenzylideneacetone)dipalladium(0) (Pd₂(dba)₃; 1.67 mmol), tri-o-tolylphosphine (P(o-tol)₃, 0.83 mmol), Et₃N (3.48 mmol) and finally acrylic acid (1.67 mmol). The resulting solution was degassed and purged several times with N₂ then heated overnight at 100° C. The solution was filtrated through a Celite pad with a fritted glass funnel then the filtrate was evaporated. The residue was purified by flash chromatography using CH₂Cl₂/MeOH (95:5) as solvent mixture yielding the title compound 8.

Step 2: N-Hydroxy-3-[4-(benzenesulfonylamino)-phenyl]-2-propenamide

(Compound 3)

The acid 8 was coupled with o-phenylenediamine in the presence of benzotriazol-1-yloxytris(dimethylamino)phosphonium hexafluorophosphate (BOP) to afford the anilide Compound 3.

¹H NMR: (300 MHz, DMSO d₆): δ 7.77 (broad s, 4H); 7.57 (d, 1H, J=15.7 Hz); 7.35 (d, 1H, J=6.9 Hz); 7.03-6.94 (m, 6H); 6.76 (d, 1H, J=7.1 Hz); 6.59 (d, 1H, J=6.9Hz); 4.98 (broad s, 2H); 2.19 (s, 3H).

¹³C NMR: (75 MHz, DMSO d₆): δ 162.9; 141.6; 139.8; 139.0; 137.6; 134.8; 133.6; 129.6; 128.1; 127.3; 125.9; 125.4; 124.7; 123.2; 120.7; 116.2; 115.9; 20.3.

### Example: 16

### Synthesis of Small Molecule No. Compound 1

The following provides a synthesis scheme for small molecule Compound No. 1 from Table 2.

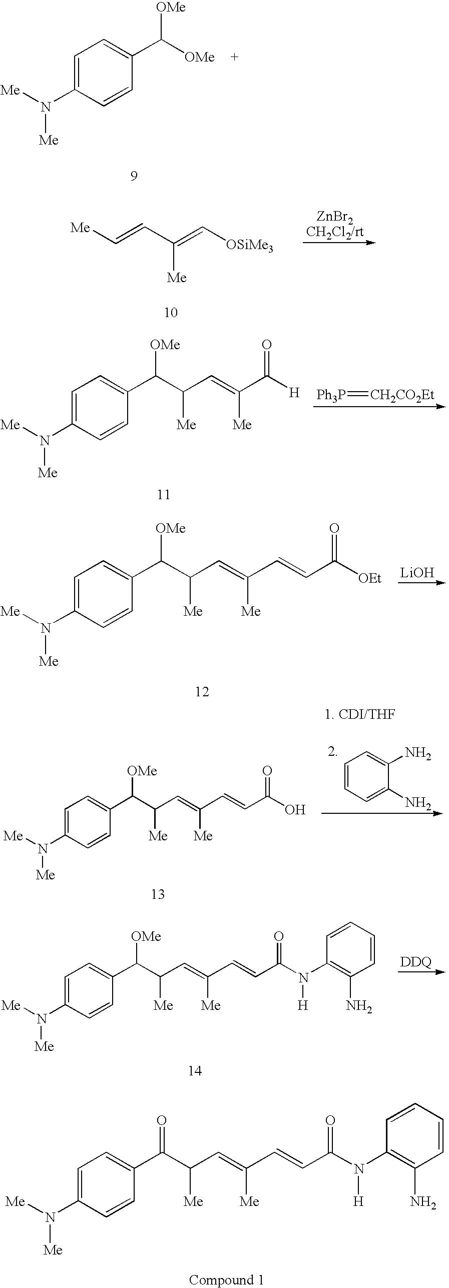

Step 1: (11)

To a stirred solution of p-anisaldehyde dimethyl acetal (9) (10 mmol) in dry CH2Cl₂ (60 mL) at rt was added 2-methyl-1-trimethylsilyloxypenta-1,3-diene (10) (Tetrahedron, 39: 881 (1983)) (10 mmol) followed by catalytic amount of anhydrous ZnBr₂ (25 mg). After being stirred for 5 h at rt, the reaction was quenched with water (20 mL). The two phases were separated and the aqueous layer was extracted with CH₂Cl₂ (2×25 mL). The combined organic layers were washed with brine, dried over magnesium sulfate, filtered, and concentrated under reduced pressure. Purification of the crude product by flash silica gel chromatography (25% ethyl acetate in hexane) afforded the desired aldehyde 11 in 68% yield as a mixture of two isomers in a ca. 2.5: 1 ratio: major isomer: ¹H NMR (300 MHz, CDCl₃) δ 9.29 (s, 1H), 7.08 (d, J=8.4 Hz, 2H), 6.67 (d, J=8.4 Hz, 2H), 6.29 (dq, J=9.9, 1.2 Hz, 1H), 3.96 (d, J=6.6 Hz, 1H), 3.20 (s, 3H), 3.05 (m, 1H), 2.94 (s, 6H), 1.60 (d, J=0.9 Hz, 3H), 1.12 (d, J=6.9 Hz, 3H).

Step 2: (12)

A mixture of aldehyde 11 (5.14 mmol) and ethyl (triphenylphosphor-anylidene)acetate (2.15 g, 6.16 mmol) in toluene (25 mL) was heated at reflux overnight under N₂. After removal of the solvent under reduced pressure, the crude product obtained was purified by flash silica gel chromatography (10% ethyl acetate in hexane) to give the title compound 12 in 96% yield as a mixture of two isomers in a ca. 2.5: 1 ratio: major isomer: ¹H NMR (300 MHz, CDCl₃) δ 7.21 (dd, J=15.6, 0.9 Hz, 1H), 7.06 (d, J=8.7 Hz, 2H), 6.66 (d, J=8.7 Hz, 2H), 5.69 (d, J=15.6 Hz, 1H), 5.67 (br. d, J=9.0 Hz, 1H), 4.17 (q, J=7.2 Hz, 2H), 3.87 (d, J=6.9 Hz, 1H), 3.18 (s, 3H), 2.93 (s, 6H), 2.81 (m, 1H), 1.59 (d, J=1.2 Hz, 3H), 1.27 (t, J=7.2 Hz, 3H), 1.05 (d, 6.6 Hz, 3H).

Step 3: (13)

To a stirred solution of diene ester 12 (1.24 mmol) in methanol (10 mL) at rt was added aqueous LiOH 0.5 N solution (1.7 mmol). After being stirred at 40° C. for 16 h, methanol was removed under reduced pressure and the resulting aqueous solution was acidified with 3N HCl (pH=ca. 4), extracted with ethyl acetate (25×3 mL), dried (MgSO₄), and concentrated under reduced pressure to give the desired carboxylic acid 13 in 98% yield: major isomer: ¹H NMR (300 MHz, CD₃OD) δ 7.21 (d, J=15.6, 0.6 Hz, 1H), 7.04 (d, J=8.7 Hz, 2H), 6.70 (d, J=8.7 Hz, 2H), 5.61 (d, J=15.6 Hz, 1 Hz, 1H), 5.60 (br. d, J=10.0 Hz, 1H), 3.85 (d, J=7.5 Hz, 1H), 3.13 (s, 3H), 2.87 (s, 6H), 2.81 (m, 1H), 1.52 (d, J=1.5 Hz, 3H), 1.06 (d, J=6.6 Hz, 3H).

Step 4: (14)

To a solution of carboxylic acid 13 (0.753 mmol) in anhydrous THF (10 mL) was added 1,1′-carbonyldiimidazole (0.790 mmol) at rt, and the mixture was stirred overnight. To the resulting solution was added 1,2-phenylenediamine (5.27 mmol), followed by trifluoroacetic acid (52 μl), and the reaction mixture was stirred for 16 h at rt. The reaction mixture was diluted with ethyl acetate (30 mL), washed with saturated NaHCO₃ solution (5 mL) and then water (10 mL), dried (MgSO₄), and concentrated. Purification by flash silica gel chromatography (50% ethyl acetate in toluene) afforded the title compound 14 in 61% yield, as a mixture of two isomers in a ca.3:1 ratio: major isomer: ¹H NMR (300 MHz, CD₃OD) δ 7.28-7.02 (m, 5H), 6.79 (m, 2H), 6.68 (d, J=8.7 Hz, 2H), 5.83 (d, J=15.0 Hz, 1H), 5.69 (d, J=9.6 Hz, 1H), 3.87 (d, J=6.9 Hz, 3.19 (s, 3H), 2.94 (s, 6H), 2.80 (m, 1H), 1.61 (br. s, 3H), 1.07 (d, J=6.6 Hz, 3H).

Step 5: (Compound 1)

To a stirred solution of compound 14 (0.216 mmol) in wet benzene (2 mL, benzene: H₂O=9: 1) at room temperature was added 2,3-dichloro-5,6-dicyano-1,4-benzoquinone (DDQ, 0.432 mmol). After being stirred vigorously for 15 min., the mixture was diluted with ethyl acetate (30 mL), washed with water (2×5 mL), dried (anhydr.MgSO₄), and concentrated. Purification by flash silica gel chromatography (50% ethyl acetate in hexanes, and then ethyl acetate only) afforded the title compound 35 (6 mg, 7% yield): ¹H NMR (300 MHz, CDCl₃) δ 7.83 (d, J=9.0, 2H), 7.87 (br. s, 1H), 7.29 (d, J=15.6 Hz, 1H), 7.27 (d, 7.8 Hz, 1H), 7.00 (m, 1H), 6.72 (m, 2H), 6.62 (d, J=9.0 Hz, 2H), 5.97 (d, J=15.6 Hz, 1H), 5.97 (d, J=9.3 Hz, 1H), 4.34 (dq, J=9.3, 6.9 Hz, 1H), 3.03 (s, 3H), 1.87 (br. s, 3H), 1.29 (d, J=6.9 Hz, 3H); ¹³C NMR (75 MHz, CDCl₃) δ 12.6, 17.6, 39.9, 40.8, 110.7, 118.0, 119.0, 119.3, 123.8, 124.4, 125.1, 126.9, 130.6, 132.5, 140.8, 146.2, 153.4, 164.8, 198.6.

### EQUIVALENTS

Those skilled in the art will recognize, or be able to ascertain, using no more than routine experimentation, many equivalents to the specific embodimemts of the invention described herein. Such equivalents are intended to be encompasssed by the following claims.

What is claimed is:

1. An agent that inhibits one or more specific histone deacetylase isoforms, but less than all histone deacetylase isoforms.

2. The agent according to claim 1, wherein the agent that inhibits one or more specific histone deacetylase isoforms, but less than all histone deacetylase isoforms, is an oligonucleotide.

3. The oligonucletide according to claim 2, wherein the oligonucleotide is complementary to a region of RNA or double-stranded DNA that encodes a portion of one or more histone deacetylase isoforms.

4. The oligonucleotide according to claim 3, wherein the oligonucleotide is a chimeric oligonucleotide.

5. The oligonucleotide according to claim 3, wherein the oligonucleotide is a hybrid oligonucleotide.

6. The oligonucleotide according to claim 3, wherein the oligonucleotide is complementary to a region of RNA or double-stranded DNA selected from the group consisting of
(a) a nucleic acid molecule encoding a portion of HDAC-1 (SEQ ID NO:2),
(b) a nucleic acid molecule encoding a portion of HDAC-2 (SEQ ID NO:4),
(c) a nucleic acid molecule encoding a portion of HDAC-3 (SEQ ID NO:6),
(d) a nucleic acid molecule encoding a portion of HDAC-4 (SEQ ID NO:8),
(e) a nucleic acid molecule encoding a portion of HDAC-5 (SEQ ID NO:10),
(f) a nucleic acid molecule encoding a portion of HDAC-6 (SEQ ID NO:12),
(g) a nucleic acid molecule encoding a portion of HDAC-7 (SEQ ID NO:14), and
(h) a nucleic acid molecule encoding a portion of HDAC-8 (SEQ ID NO:18).

7. The oligonucleotide according to claim 6 having a nucleotide sequence of from about 13 to about 35 nucleotides.

8. The oligonucleotide according to claim 6 having a nucleotide sequence of from about 15 to about 26 nucleotides.

9. The oligonucleotide according to claim 6 having one or more phosphorothioate internucleoside linkage, being 20-26 nucleotides in length, and being modified such that the terminal four nucleotides at the 5′ end of the oligonucleotide and the terminal four nucleotides at the 3′ end of the oligonucleotide each have 2′-O-methyl groups attached to their sugar residues.

10. The oligonucleotide according to claim 6, wherein the oligonucleotide is complementary to a region of RNA or double-stranded DNA encoding a portion of HDAC-1 (SEQ ID NO:2).

11. The oligonucleotide according to claim 10 that is SEQ ID NO:17 or SEQ ID NO:18.

12. The oligonucleotide according to claim 6, wherein the oligonucleotide is complementary to a region of RNA or double-stranded DNA encoding a portion of HDAC-2 (SEQ ID NO:4).

13. The oligonucleotide according to claim 12 that is SEQ ID NO:20.

14. The oligonucleotide according to claim 6, wherein the oligonucleotide is complementary to a region of RNA or double-stranded DNA encoding a portion of HDAC-3 (SEQ ID NO:6).

15. The oligonucleotide according to claim 14 that is SEQ ID NO:22.

16. The oligonucleotide according to claim 6, wherein the oligonucleotide is complementary to a region of RNA or double-stranded DNA encoding a portion of HDAC-4 (SEQ ID NO:8).

17. The oligonucleotide according to claim 16 that is SEQ ID NO:24 or 26.

18. The oligonucleotide according to claim 6, wherein the oligonucleotide is complementary to a region of RNA or double-stranded DNA encoding a portion of HDAC-5 (SEQ ID NO:10).

19. The oligonucleotide according to claim 18 that is SEQ ID NO:28.

20. The oligonucleotide according to claim 6, wherein the oligonucleotide is complementary to a region of RNA or double-stranded DNA encoding a portion of HDAC-6 (SEQ ID NO:12).

21. The oligonucleotide according to claim 20 that is SEQ ID NO:29.

22. The oligonucleotide according to claim 6, wherein the oligonucleotide is complementary to a region of RNA or double-stranded DNA encoding a portion of HDAC-7 (SEQ ID NO:14).

23. The oligonucleotide according to claim 22 that is SEQ ID NO:31.

24. The oligonucleotide according to claim 6, wherein the oligonucleotide is complementary to a region of RNA or double-stranded DNA encoding a portion of HDAC-8 (SEQ ID NO:16).

25. The oligonucleotide according to claim 24 that is SEQ ID NO:32 or SEQ ID NO:33.

26. A method for inhibiting one or more histone deacetylase isoforms in a cell comprising contacting the cell with the agent according to claim 1.

27. A method for inhibiting one or more histone deacetylase isoforms in a cell comprising contacting the cell with the oligonucleotide according to claim 3.

28. The method according to claim 27, wherein cell proliferation is inhibited in the contacted cell.

29. The method according to claim 27, wherein the oligonucleotide that inhibits cell proliferation in a contacted cell induces the contacted cell to undergo growth retardation.

30. The method according to claim 27, wherein the oligonucleotide that inhibits cell proliferation in a contacted cell induces the contacted cell to undergo growth arrest.

31. The method according to claim 27, wherein the oligonucleotide that inhibits cell proliferation in a contacted cell induces the contacted cell to undergo programmed cell death.

32. The method according to claim 27, wherein the oligonucleotide that inhibits cell proliferation in a contacted cell induces the contacted cell to undergo necrotic cell death.

33. The method according to claim 27, further comprising contacting the cell with a histone deacetylase small molecule inhibitor.

34. A method for inhibiting neoplastic cell proliferation in an animal comprising administering to an animal having at least one neoplastic cell present in its body a therapeutically effective amount of the agent of claim 1.

35. A method for inhibiting neoplastic cell proliferation in an animal comprising administering to an animal having at least one neoplastic cell present in its body a therapeutically effective amount of the oligonucleotide of claim 3.

36. The method according to claim 35, wherein the animal is a human.

37. The method according to claim 35, further comprising administering to the animal a therapeutically effective amount of a histone deacetylase small molecule inhibitor with a pharmaceutically acceptable carrier for a therapeutically effective period of time.

38. A method for identifying a histone deacetylase isoform that is required for the induction of cell proliferation, the method comprising contacting the histone deacetylase isoform with an inhibitory agent, wherein a decrease in the induction of cell proliferation indicates that the histone deacetylase isoform is required for the induction of cell proliferation.

39. The method according to claim 38, wherein the inhibitory agent is an oligonucleotide of claim 3.

40. A method for identifying a histone deacetylase isoform that is required for cell proliferation, the method comprising contacting the histone deacetylase isoform with an inhibitory agent, wherein a decrease in cell proliferation indicates that the histone deacetylase isoform is required for cell proliferation.

41. The method according to claim 40, wherein the inhibitory agent is an oligonucleotide of claim 3.

42. A method for identifying a histone deacetylase isoform that is required for the induction of cell differentiation, the method comprising contacting the histone deacetylase isoform with an inhibitory agent, wherein an induction of cell differentiation indicates that the histone deacetylase isoform is required for the induction of cell proliferation.

43. The method according to claim 38, wherein the inhibitory agent is an oligonucleotide of claim 3.

44. A method for inhibiting cell proliferation in a cell, comprising contacting a cell with at least two reagents selected from the group consisting of an antisense oligonucleotide that inhibits a specific histone deacetylase isoform, a histone deacetylase small molecule inhibitor that inhibits a specific histone deacetylase isoform, an antisense oligonucleotide that inhibits a DNA methyltransferase, and a DNA methyltransferase small molecule inhibitor.

45. A method for modulating cell proliferation or differentiation of a cell comprising inhibiting a specific HDAC isoform that is involved in cell proliferation or differentiation by contacting the cell with an agent of claim 1.

46. The method according to claim 45, wherein the cell proliferation is neoplasia.

47. The method according to claim 46, wherein the histone deacetylase isoform is selected from the group consisting of HDAC-1, HDAC-2, HDAC-3, HDAC-4, HDAC-5, HDAC-6, HDAC-7 and HDAC-8.

48. The method according to claim 47, wherein the histone deacetylase isoform is HDAC-1 and/or HDAC-4.
